(19) United States
(12) Patent Application Publication
(10) Pub. No.: US 2007/0208053 A1
(43) Pub. Date: Sep. 6, 2007

(54) Fused heterobicyclic kinase inhibitors

(76) Inventors: Lee D. Arnold, Farmingdale, NY (US); Xin Chen, Farmingdale, NY (US); Hanqing Dong, Farmingdale, NY (US); Andrew Garton, Farmingdale, NY (US); Mark Joseph Mulvihill, Farmingdale, NY (US); Colin Peter Sambrook Smith, Oxford (GB); Gerard Hugh Thomas, Oxford (GB); Thomas Martin Krulle, Oxford (GB); Jing Wang, Farmingdale, NY (US)

Correspondence Address: OSI PHARMACEUTICALS, INC., 41 PINELAWN ROAD, MELVILLE, NY, 11747, US

(21) Appl. No.: 11/654,814

(22) Filed: Jan. 18, 2007

Related U.S. Application Data
(60) Provisional application No. 60/760,124, filed on Jan. 19, 2006.

(51) Int. Cl. C07D 471/02; A61K 31/4745

(52) U.S. Cl. 514/300; 546/113

(57) ABSTRACT

Compounds of the formula 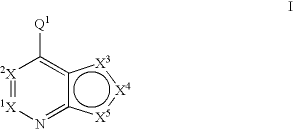
and pharmaceutically acceptable salts thereof, wherein X₁, X₂, X₃, X₄, X₅, X₅, X₇, R¹, and Q¹ are defined herein, inhibit kinase enzymes and are useful for the treatment and/or prevention of hyperproliferative diseases such as cancer. The compounds are also useful in the treatment of inflammation, allergy, asthma, disease and conditions of the immune system, disease and conditions of the nervous system, cardiovascular diseases, disease and conditions of the eye, dermatological diseases, osteoporosis, diabetes, multiple sclerosis, and infections.

This application claims the benefit of U.S. Patent Application No. 60/760,124, filed Jan. 19, 2006.

### BACKGROUND OF THE INVENTION

The present invention is directed to fused heterobicyclic compounds. In particular, the present invention is directed to fused heterobicyclic compounds that inhibit at least one of the kinases Akt, Alk, Aurora-A, CDK2, CSF-1R, EGFR, FAK, Flt3, IGF-1R, IKKb, KDR, Kit, MEK1, Met, p70S6K, PDK1, PKA, PKC, PKN1, Ret, ROCK1, ROCK2, RON, RSK1, or SGK, and are useful in the treatment of inflammation, cancer, allergy, asthma, disease and conditions of the immune system, disease and conditions of the nervous system, cardiovascular disease, dermatological diseases, osteoporosis, metabolic diseases including diabetes, multiple sclerosis, ocular diseases and angiogenesis, viral infections and bacterial infections

Such cardiovascular diseases include hypertension, vasospasm, preterm labor, atherosclerosis, myocardial hypertrophy, erectile dysfunction, restenosis. Ocular diseases include glaucoma, diabetic retinopathy, choroidal neovascularization due to age-related macular degeneration, retinopathy of prematurity. Cancers include vascular smooth muscle cell hyperproliferation, bladder cancer, pancreatic cancer, testicular cancer, colon cancer, lung cancer, breast cancer, prostate cancer, hepatocellular carcinoma, melanoma, ovarian cancer, sarcoma and other hyperproliferative disorders. Cancer treatment includes reducing the extent of metastatic spread of cancer cells from the primary tumor site to distant organs and tissues. Cancer treatment includes reducing the transition of cancer cells of epithelial origin to mesenchymal-like cells through the process of epithelial-mesenchymal transition. Cancer treatment includes limiting the toxicity of cytotoxics which act in S-phase, G2 or mitosis. Cancer treatment include limiting angiogenic processes or the formation of vascular hyperpermeability that lead to edema, ascites, effusions, exudates, and macromolecular extravasation and matrix deposition. Inflammatory diseases include endothelial dysfunction inflammation, arthritis, rheumatoid arthritis, nervous system conditions and diseases include neurological diseases, neurodegenerative disorders, stroke, Alzheimer's disease. Disease and conditions of the immune system include autoimmune disorders, allograft rejection, and graft vs. host disease, AIDS, hyper-immune responses. Dermatologic diseases include psoriasis, infantile hemangiomas. Viral infection treatment includes disrupting the virus life cycle by preventing virus replication. Bacterial infection treatment includes inhibition of invasion of bacteria into epithelial cells.

Phosphoryl transferases are a large family of enzymes that transfer phosphorous-containing groups from one substrate to another. Kinases are a class of enzymes that function in the catalysis of phosphoryl transfer. The phosphorylation is usually a transfer reaction of a phosphate group from ATP to the protein substrate. Almost all kinases contain a similar 250-300 amino acid catalytic domain. Protein kinases, with at least 400 identified, constitute the largest subfamily of structurally related phosphoryl transferases and are responsible for the control of a wide variety of signal transduction processes within the cell. The protein kinases may be categorized into families by the substrates they phosphorylate (e.g., protein-serine/threonine, protein-tyrosine etc.). Protein kinase sequence motifs have been identified that generally correspond to each of these kinase families. Lipid kinases (e.g. PI3K) constitute a separate group of kinases with structural similarity to protein kinases.

The “kinase domain” appears in a number of polypeptides that serve a variety of functions. Such polypeptides include, for example, transmembrane receptors, intracellular receptor associated polypeptides, cytoplasmic located polypeptides, nuclear located polypeptides and subcellular located polypeptides. The activity of protein kinases can be regulated by a variety of mechanisms and any individual protein might be regulated by more than one mechanism. Such mechanisms include, for example, autophosphorylation, transphosphorylation by other kinases, protein-protein interactions, protein-lipid interactions, protein-polynucleotide interactions, ligand binding, and post-translational modification.

Phosphorylation of target proteins occurs in response to a variety of extracellular signals (hormones, neurotransmitters, growth and differentiation factors, etc.), cell cycle events, environmental or nutritional stresses, etc. Protein and lipid kinases regulate many different cell processes by adding phosphate groups to targets such as proteins or lipids. Such cell processes include, for example, proliferation, growth, differentiation, metabolism, cell cycle events, apoptosis, motility, transcription, translation and other signaling processes. Kinase catalyzed phosphorylation acts as molecular on/off switches to modulate or regulate the biological function of the target protein. Thus, protein and lipid kinases can function in signaling pathways to activate or inactivate, or modulate the activity (either directly or indirectly) of the targets. These targets may include, for example, metabolic enzymes, regulatory proteins, receptors, cytoskeletal proteins, ion channels or pumps, or transcription factors.

A partial list of protein kinases includes abl, AKT, Alk, Aurora-A, bcr-abl, Blk, Brk, Btk, c-kit, c-met, c-src, CDK1, CDK2, CDK3, CDK4, CDKS, CDK6, CDK7, CDK8, CDK9, CDK10, cRaf1, CSF1r, CSK, EGFR, ErbB2, ErbB3, ErbB4, Erk, Fak, fes, FGFR1, FGFR2, FGFR3, FGFR4, FGFR5, Fgr, flt-1, Flt3, Fps, Frk, Fyn, Hck, IGF-1R, IKKβ, INS-R, Jak, KDR, Lck, Lyn, MEK, Met, MYLK2, p38, p70S6K, PDGFR, PDK1, PIK, PKA, PKC, PKN, PYK2, Ret, ron, Rsk1, SGK, tie, tie2, TRK, Yes, and Zap70. Thus, protein kinases represent a large family of proteins that play a central role in the regulation of a wide variety of cellular processes, maintaining control over cellular function. Uncontrolled signaling due to defective control of protein phosphorylation has been implicated in a number of diseases and disease conditions, including, for example, inflammation, cancer, allergy/asthma, disease and conditions of the immune system, disease and conditions of the central nervous system (CNS), cardiovascular disease, dermatology, ocular diseases and angiogenesis.

Inappropriately high protein kinase activity has been implicated in many diseases resulting from abnormal cellular function. This might arise either directly or indirectly, by failure of the proper control mechanisms for the kinase, related to mutation, over-expression or inappropriate activation of the enzyme; or by over- or underproduction of cytokines or growth factors also participating in the transduction of signals upstream or downstream of the kinase. In all of these instances, selective inhibition of the action of the kinase can have a beneficial effect.

Initial interest in protein kinases as pharmacological targets was stimulated by findings that many viral oncogenes encode structurally modified cellular protein kinases with constitutive enzyme activity. One early example was the Rous sarcoma virus (RSV) or avian sarcoma virus (ASV), which caused highly malignant tumors of the same type or sarcomas within infected chickens. Subsequently, deregulated protein kinase activity, resulting from a variety of mechanisms, has been implicated in the pathophysiology of a number of important human disorders including, for example, cancer, CNS conditions, and immunologically related diseases. The development of selective protein kinase inhibitors that can block the disease pathologies and/or symptoms resulting from aberrant protein kinase activity has therefore become an important therapeutic target.

The Ser/Thr protein kinase family of enzymes comprises more than 400 members including 6 major subfamilies (AGC, CAMK, CMGC, GYC, TKL, STE). Many of these enzymes are considered targets for pharmaceutical intervention in various disease states.

ROCK1 and ROCK2 (rho-associated coiled-coil containing kinase-1 and -2, also known as Rokβ/p160ROCK and Rokα, respectively) are closely related members of the AGC subfamily of enzymes that are activated downstream of activated rho in response to a number of extracellular stimuli, including growth factors, integrin activation and cellular stress (Riento and Ridley, Nature Reviews Molecular Cell Biology, 4: 446-456 (2003)). As used herein unless specifically identified as ROCK1 or ROCK2, the term “ROCK” will mean one of, or both of, the ROCK1 and ROCK2 isoforms. The ROCK enzymes play key roles in multiple cellular processes including cell morphology, stress fiber formation and function, cell adhesion, cell migration and invasion, epithelial-mesenchymal transition (EMT), transformation, phagocytosis, apoptosis, neurite retraction, cytokinesis and mitosis and cellular differentiation (Riento and Ridley, Nature Reviews Molecular Cell Biology, 4: 446-456 (2003)). As such, ROCK kinases represent potential targets for development of inhibitors to treat a variety of disorders, including cancer, hypertension, vasospasm, asthma, preterm labor, erectile dysfunction, glaucoma, vascular smooth muscle cell hyperproliferation, atherosclerosis, myocardial hypertrophy, endothelial dysfunction and neurological diseases (Wettschurek and Offermanns, J Molecular Medicine, 80: 629-638 (2002); Mueller et al., Nature Reviews Drug Discovery, 4: 387-398 (2005), Sahai and Marshall, Nature Reviews Cancer, 2: 133-142 (2002)).

Inhibition of ROCK activity reduces cell migration and reduces metastasis of tumor cells in vivo (Somlyo et al., Biochem Biophys Res Commun, 269: 6562-659 (2000); Somlyo et al., FASEB J, 17: 223-234 (2003); Genda et al., Hepatology, 30: 1027-1036 (1999; Takamura et al., Hepatology, 33: 577-581 (2001); Nakajima et al., Eur J Pharmacology, 459: 113-120 (2003); Nakaijima et al., Cancer Chemother Pharmacol, 52: 319-324 (2003); Itoh et al., Nature Medicine, 5: 221-225 (1999)). Overexpression of ROCK has been associated with invasion and metastasis in clinical samples derived from bladder cancer patients (Kamai et al., Clinical Cancer Research, 9: 2632-2641 (2003)) and ROCK protein is overexpressed in pancreatic cancer (Pancreas, 24: 251-257 (2002) and testicular cancer (Clin Cancer Res 10, 4799-4805 (2004)). Expression of constitutively active ROCK2 in colon cancer cells induced tumor dissemination into the surrounding stroma and increased tumor vascularity (Croft et al., Cancer Research 64, 8994-9001 (2004)). ROCK enzymes are involved in the transition of cells from an epithelial to mesenchymal phenotype (Bhowmick et al., Mol Biol Cell 12, 27-36 (2001)), a process thought to be important for progression of tumors towards a more malignant metastatic phenotype (Thiery, Nature Reviews Cancer, 2: 442-454 (2002)).

Cdc2 (cdk1)/cyclin B is another serine/threonine kinase enzyme which belongs to the cyclin-dependent kinase (cdks) family. These enzymes are involved in the critical transition between various phases of cell cycle progression. It is believed that uncontrolled cell proliferation, the hallmark of cancer, is dependent upon elevated cdk activities in these cells. The loss of control of cdk regulation is a frequent event in hyperproliferative diseases and cancer (Pines, Current Opinion in Cell Biology, 4:144-148 (1992); Lees, Current Opinion in Cell Biology, 7:773-780 (1995); Hunter and Pines, Cell, 79:573-582 (1994)). The inhibition of elevated cdk activities in cancer cells by cdc2/cyclin B kinase inhibitors could suppress proliferation and may restore the normal control of cell cycle progression.

Protein tyrosine kinases (PTKs) are enzymes that catalyse the phosphorylation of specific tyrosine residues in cellular proteins. Such post-translational modification of the substrate proteins, often enzymes themselves, acts as a molecular switch regulating cell proliferation, activation or differentiation (for review, see Schlessinger and Ullrich, 1992, Neuron 9:383-391). Aberrant or excessive PTK activity has been observed in many disease states including benign and malignant proliferative disorders as well as diseases resulting from inappropriate activation of the immune system (e.g., autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, infantile hemangiomas).

Tyrosine kinases can be of the receptor-type (having extracellular, transmembrane and intracellular domains) or the non-receptor type (being wholly intracellular). The Receptor Tyrosine Kinases (RTKs) comprise a large family of transmembrane receptors with at least nineteen distinct RTK subfamilies having diverse biological activities. The RTK family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, RTK mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response such as cell division, differentiation, metabolic effects, and changes in the extracellular microenvironment (see Schlessinger and Ullrich, 1992, Neuron 9:1-20).

Proteins with SH2 (src homology -2) or phosphotyrosine binding (PTB) domains bind activated tyrosine kinase receptors and their substrates with high affinity to propagate signals into cell. Both of the domains recognize phosphotyrosine. (Fantl et al., 1992, Cell 69:413-423; Songyang et al., 1994, Mol. Cell. Biol. 14:2777-2785; Songyang et al., 1993, Cell 72:767-778; and Koch et al., 1991, Science 252:668-678; Shoelson, Curr Opin. Chem. Biol. (1997), 1(2), 227-234; Cowburn, Curr Opin. Struct. Biol. (1997), 7(6), 835-838). Several intracellular substrate proteins that associate with RTKs have been identified. They may be divided into two principal groups: (1) substrates which have a catalytic domain; and (2) substrates which lack such a domain but serve as adapters and associate with catalytically active molecules (Songyang et al., 1993, Cell 72:767-778). The specificity of the interactions between receptors or proteins and SH2 or PTB domains of their substrates is determined by the amino acid residues immediately surrounding the phosphorylated tyrosine residue. For example, differences in the binding affinities between SID domains and the amino acid sequences surrounding the phosphotyrosine residues on particular receptors correlate with the observed differences in their substrate phosphorylation profiles (Songyang et al., 1993, Cell 72:767-778). Observations suggest that the function of each receptor tyrosine kinase is determined not only by its pattern of expression and ligand availability but also by the array of downstream signal transduction pathways that are activated by a particular receptor as well as the timing and duration of those stimuli. Thus, phosphorylation provides an important regulatory step, which determines the selectivity of signaling pathways recruited by specific growth factor receptors, as well as differentiation factor receptors.

Several receptor tyrosine kinases such as FGFR-1, PDGFR, Tie-2 and c-Met, and growth factors that bind thereto, have been suggested to play a role in angiogenesis, although some may promote angiogenesis indirectly (Mustonen and Alitalo, J. Cell Biol. 129:895-898, 1995). One such receptor tyrosine kinase, known as “fetal liver kinase 1” (FLK-1), is a member of the type III subclass of RTKs. Human FLK-1 is also known as “kinase insert domain-containing receptor” (KDR) (Terman et al., Oncogene 6:1677-83, 1991). It is also called “vascular endothelial cell growth factor receptor 2” (VEGFR-2) since it binds vascular endothelial cell growth factor (VEGF) with high affinity. The murine version of FLK-1/VEGFR-2 has also been called NYK. (Oelrichs et al, Oncogene 8(1):11-15, 1993). Numerous studies (such as those reported in Millauer et al., supra), suggest that VEGF and FLK-1/KDR/VEGFR-2 are a ligand-receptor pair that play an important role in the proliferation of vascular endothelial cells (vasculogenesis), and the formation and sprouting of blood vessels (angiogenesis). Accordingly, VEGF plays a role in the stimulation of both normal and pathological angiogenesis (Jakeman et al., Endocrinology 133:848-859, 1993; Kolch et al., Breast Cancer Research and Treatment 36: 139-155, 1995; Ferrara et al., Endocrine Reviews 18(1); 4-25, 1997; Ferrara et al., Regulation of Angiogenesis (ed. L D. Goldberg and E. M. Rosen), 209-232,1997). In addition, VEGF has been implicated in the control and enhancement of vascular permeability (Connolly, et al., 1. Biol. Chem. 264: 20017-20024, 1989; Brown et al., Regulation of Angiogenesis (ed. L D. Goldberg and E. M. Rosen), 233-269, 1997).

Another type III subclass RTK related to FLK-1/KDR (DeVries et al. Science 255:989-991, 1992; Shibuya et al., Oncogene 5:519-524, 1990) is “fins-like tyrosine kinase-I” (Flt-1), also called “vascular endothelial cell growth factor receptor 1” (VEGFR-1). Members of the FLK-1/KDR/VEGFR-2 and Flt-1/VEGPR-1 subfamilies are expressed primarily on endothelial cells. These subclass members are specifically stimulated by members of the VEGF family of ligands (Klagsbum and D'Amore, Cytokine & Growth Factor Reviews 7: 259270, 1996). VEGF binds to Flt-1 with higher affinity than to FLK-1/KDR and is mitogenic toward vascular endothelial cells (Terman et al., 1992, supra; Mustonen et al. supra; DeVries et al., supra). Flt-1 is believed to be essential for endothelial organization during vascular development. Flt-1 expression is associated with early vascular development in mouse embryos, and with neovascularization during wound healing (Mustonen and Alitalo, supra). Expression of Flt-1 in monocytes, osteoclasts, and osteoblasts, as well as in adult tissues such as kidney glomeruli suggests an additional function for this receptor that is not related to cell growth (Mustonen and Alitalo, supra).

Tie-2 (TEK) is a member of a recently discovered family of endothelial cell specific RTKs involved in critical angiogenic processes such as vessel branching, sprouting, remodeling, maturation and stability. Tie-2 is the first mammalian RTK for which both agonist ligands (e.g., Angiopoietinl (“Ang1”), which stimulates receptor autophosphorylation and signal transduction), and antagonist ligands (e.g., Angiopoietin2 (“Ang2”)), have been identified. The current model suggests that stimulation of Tie-2 kinase by the Ang1 ligand is directly involved in the branching, sprouting and outgrowth of new vessels, and recruitment and interaction of periendothelial support cells important in maintaining vessel integrity and inducing quiescence. The absence of Ang1 stimulation of Tie-2 or the inhibition of Tie-2 autophosphorylation by Ang2, which is produced at high levels at sites of vascular regression, may cause a loss in vascular structure and matrix contacts resulting in endothelial cell death, especially in the absence of growth/survival stimuli. Recently, significant upregulation of Tie-2 expression has been found within the vascular synovial pannus of arthritic joints of humans, consistent with a role in the inappropriate neovascularization, suggesting that Tie-2 plays a role in the progression of rheumatoid arthritis. Point mutations producing constitutively activated forms of Tie-2 have been identified in association with human venous malformation disorders. Tie-2 inhibitors are, therefore, useful in treating such disorders, and in other situations of inappropriate neovascularization.

Non-receptor tyrosine kinases represent a collection of cellular enzymes that lack extracellular and transmembrane sequences (see, Bohlen, 1993, Oncogene 8:2025-2031). Over twenty-four individual non-receptor tyrosine kinases, comprising eleven (11) subfamilies have been identified. The Src subfamily of non-receptor tyrosine kinases is comprised of the largest number of PTKs and includes Src, Yes, Fyn, Lyn, Lck, Blk, Hck, Fgr and Yrk. The Src subfamily of enzymes has been linked to oncogenesis and immune responses.

Focal adhesion kinase (FAK) is a protein that is localized to sites of cell adhesion (focal contacts) and FAK is necessary for cellular transformation by the oncogene src. FAK is a cytosolic tyrosine kinase that controls cell shape, cell motility and adhesion to the extracellular matrix. FAK integrates signals from integrin receptors, growth factor receptor tyrosine kinases (RTKs) and G protein-coupled receptors to promote cell migration in response to extracellular stimuli. FAK also mediates pro-survival signals in response to anchorage independence as well as endothelial cell migration, important in tumor angiogenesis. FAK mRNA is increased in many human carcinomas and FAK protein over-expression is associated with advanced malignancies. Given its strong involvement in controlling processes relevant to tumor development like motility, migration and tumor cell survival, FAK is considered to be an attractive target for the development of anti-cancer therapeutic agents (McLean et al., Nat Rev Cancer. 2005 5: 505-15 (2005); Mitra et al., Nat Rev Mol Cell Biol. 6: 56-68 (2005); Avizienyte et al., Curr Opin Cell Biol. 17: 542 (2005).

Malignant cells are associated with the loss of control over one or more cell cycle elements. These elements range from cell surface receptors to the regulators of transcription and translation, including the insulin-like growth factors, insulin growth factor-1 (IGF-1) and insulin growth factor-2 (IGF-2). [M. J. Ellis, “The Insulin-Like Growth Factor Network and Breast Cancer”, Breast Cancer, Molecular Genetics, Pathogenesis and Therapeutics, Humana Press 1999]. The insulin growth factor system consists of families of ligands, insulin growth factor binding proteins, and receptors.

A major physiological role of the IGF-1 system is the promotion of normal growth and regeneration, and overexpressed IGF-1R can initiate mitogenesis and promote ligand-dependent neoplastic transformation. Furthermore, IGF-1R plays an important role in the establishment and maintenance of the malignant phenotype.

IGF-1R exists as a heterodimer, with several disulfide bridges. The tyrosine kinase catalytic site and the ATP binding site are located on the cytoplasmic portion of the beta subunit. Unlike the epidermal growth factor (EGF) receptor, no mutant oncogenic forms of the IGF-1R have been identified. However, several oncogenes have been demonstrated to affect IGF-1 and IGF-1R expression. The correlation between a reduction of IGF-1R expression and resistance to transformation has been seen. Exposure of cells to the mRNA antisense to IGF-1R RNA prevents soft agar growth of several human tumor cell lines.

IGF-1R performs important roles in cell division, development, and metabolism, and in its activated state, plays a role in oncogenesis and suppression of apoptosis. IGF-1R is known to be overexpressed in a number of cancer cell lines (IGF-1R overexpression is linked to acromegaly and to cancer of the prostate). By contrast, down-regulation of IGF-1R expression has been shown to result in the inhibition of tumorigenesis and an increased apoptosis of tumor cells.

Apoptosis is a ubiquitous physiological process used to eliminate damaged or unwanted cells in multicellular organisms. Disregulation of apoptosis is believed to be involved in the pathogenesis of many human diseases. The failure of apoptotic cell death has been implicated in various cancers, as well as autoimmune disorders. Conversely, increased apoptosis is associated with a variety of diseases involving cell loss such as neurodegenerative disorders and AIDS. As such, regulators of apoptosis have become an important therapeutic target. It is now established that a major mode of tumor survival is escape from apoptosis. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The type 1 insulin-like growth factor receptor (IGF-1R) is a transmembrane RTK that binds primarily to IGF-1 but also to IGF-II and insulin with lower affinity. Binding of IGF-1 to its receptor results in receptor oligomerization, activation of tyrosine kinase, intermolecular receptor autophosphorylation and phosphorylation of cellular substrates (major substrates are IRS1 and Shc). The ligand-activated IGF-1R induces mitogenic activity in normal cells and plays an important role in abnormal growth.

Several clinical reports underline the important role of the IGF-1 pathway in human tumor development: 1) IGF-1R overexpression is frequently found in various tumors (breast, colon, lung, sarcoma.) and is often associated with an aggressive phenotype. 2) High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models.

Many of the tyrosine kinases, whether an RTK or non-receptor tyrosine kinase, have been found to be involved in cellular signaling pathways involved in numerous pathogenic conditions, including cancer, psoriasis, and other hyperproliferative disorders or hyper-immune responses. Therefore, much research is ongoing for inhibitors of kinases involved in mediating or maintaining disease states to treat such diseases. Examples of such kinase research include, for example: (1) inhibition of c-Src (Brickell, Critical Reviews in Oncogenesis, 3:401-406 (1992); Courtneidge, Seminars in Cancer Biology, 5:236-246 (1994), raf (Powis, Pharmacology & Therapeutics, 62:57-95 (1994)) and the cyclin-dependent kinases (CDKs) 1, 2 and 4 in cancer (Pines, Current Opinion in Cell Biology, 4:144-148 (1992); Lees, Current Opinion in Cell Biology, 7:773-780 (1995); Hunter and Pines, Cell, 79:573-582 (1994)), (2) inhibition of CDK2 or PDGF-R kinase in restenosis (Buchdunger et al., Proceedings of the National Academy of Science USA, 92:2258-2262 (1995)), (3) inhibition of CDK5 and GSK3 kinases in Alzheimer's (Hosoi et al., Journal of Biochemistry (Tokyo), 117:741-749 (1995); Aplin et al., Journal of Neurochemistry, 67:699-707 (1996), (4) inhibition of c-Src kinase in osteoporosis (Tanaka et al., Nature, 383:528-531 (1996), (5) inhibition of GSK-3 kinase in type-2 diabetes (Borthwick et al., Biochemical & Biophysical Research Communications, 210:738-745 (1995), (6) inhibition of the p38 kinase in inflammation (Badger et al., The Journal of Pharmacology and Experimental Therapeutics, 279:1453-1461 (1996)), (7) inhibition of VEGF-R1-3 and TIE-1 and 2 kinases in diseases which involve angiogenesis (Shawver et al., Drug Discovery Today, 2:50-63 (1997)), (8) inhibition of UL97 kinase in viral infections (He et al., Journal of Virology, 71:405-411 (1997)), (9) inhibition of CSF-1R kinase in bone and hematopoetic diseases (Myers et. al., Bioorganic & Medicinal Chemistry Letters, 7:421-424 (1997), and (10) inhibition of Lck kinase in autoimmune diseases and transplant rejection (Myers et. al., Bioorganic & Medicinal Chemistry Letters, 7:417-420 (1997)).

Inhibitors of certain kinases may be useful in the treatment of diseases when the kinase is not misregulated, but it nonetheless essential for maintenance of the disease state. In this case, inhibition of the kinase activity would act either as a cure or palliative for these diseases. For example, many viruses, such as human papilloma virus, disrupt the cell cycle and drive cells into the S-phase of the cell cycle (Vousden, FASEB Journal, 7:8720879 (1993)). Preventing cells from entering DNA synthesis after viral infection by inhibition of essential S-phase initiating activities such as CDK2, may disrupt the virus life cycle by preventing virus replication. This same principle may be used to protect normal cells of the body from toxicity of cycle-specific chemotherapeutic agents (Stone et al., Cancer Research, 56:3199-3202 (1996); Kohn et al., Journal of Cellular Biochemistry, 54:44-452 (1994). Inhibition of CDK 2 or 4 will prevent progression into the cycle in normal cells and limit the toxicity of cytotoxics, which act in S-phase, G2 or mitosis.

Furthermore, CDK2/cyclin E activity has also been shown to regulate NF-kB. Inhibition of CDK2 activity stimulates NF-kB-dependent gene expression, an event mediated through interactions with the p300 co-activator (Perkins et al., Science, 275:523-527 (1997)). NF-kB regulates genes involved in inflammatory responses (such as hematopoetic growth factors, chemokines and leukocyte adhesion molecules) (Baeuerle and Henkel, Annual Review of Immunology, 12:141-179 (1994)) and maybe involved in the suppression of apoptotic signals within the cell (Beg and Baltimore, Science, 274:782-784 (1996); Wang et al., Science, 274:784-787 (1996); Van Antwerp et al., Science, 274:787-789 (1996). Thus, inhibition of CDK2 may suppress apoptosis induced by cytotoxic drugs via a mechanism that involves NF-kB and be useful where regulation of NF-kB plays a role in etiology of disease.

The identification of effective small compounds which specifically inhibit signal transduction and cellular proliferation by modulating the activity of receptor and non-receptor tyrosine and serine/threonine kinases to regulate and modulate abnormal or inappropriate cell proliferation, differentiation, or metabolism is therefore desirable. In particular, the identification of methods and compounds that specifically inhibit the function of a tyrosine kinase which is essential for angiogenic processes or the formation of vascular hyperpermeability leading to edema, ascites, effusions, exudates, and macromolecular extravasation and matrix deposition as well as associated disorders would be beneficial.

In view of the importance of PTKs to the control, regulation, and modulation of cell proliferation and the diseases and disorders associated with abnormal cell proliferation, many attempts have been made to identify receptor and non-receptor tyrosine kinase inhibitors using a variety of approaches, including the use of mutant ligands (U.S. Pat. No. 4,966,849), soluble receptors and antibodies (International Patent Publication No. WO 94/10202; Kendall & Thomas, 1994, Proc. Natl. Acad. Sci. 90:10705-09; Kim et al., 1993, Nature 362:841-844), RNA ligands (Jellinek, et al., Biochemistry 33:1045056; Takano, et al., 1993, Mol. Bio. Cell 4:358 A; Kinsella, et al. 1992, Exp. Cell Res. 199:56-62; Wright, et al., 1992,1. Cellular Phys. 152:448-57) and tyrosine kinase inhibitors (International Patent Publication Nos. WO 94/03427; WO 92/21660; WO 91/15495; WO 94/14808; U.S. Pat. No. 5,330,992; Mariani, et al., 1994, Proc. Am. Assoc. Cancer Res. 35:2268).

More recently, attempts have been made to identify small molecules that act as tyrosine kinase inhibitors. Bis-, monocyclic, bicyclic or heterocyclic aryl compounds (International Patent Publication No. WO 92/20642) and vinylene-azaindole derivatives (International Patent Publication No. WO 94/14808) have been described generally as tyrosine kinase inhibitors. Styryl compounds (U.S. Pat. No. 5,217,999), styryl-substituted pyridyl compounds (U.S. Pat. No. 5,302,606), certain quinazoline derivatives (EP Application No. 0566266 A1; Expert Opin. Ther. Pat. (1998), 8(4): 475-478), selenoindoles and selenides (International Patent Publication No. WO 94/03427), tricyclic polyhydroxylic compounds (International Patent Publication No. WO 92/21660) and benzylphosphonic acid compounds (International Patent Publication No. WO 91/15495) have been described as compounds for use as tyrosine kinase inhibitors for use in the treatment of cancer. Anilinocinnolines (PCT WO97/34876) and quinazoline derivative compounds (International Patent Publication No. WO 97/22596; International Patent Publication No. WO97/42187) have been described as inhibitors of angiogenesis and vascular permeability. Bis(indolylmaleimide) compounds have been described as inhibiting particular PKC serine/threonine kinase isoforms whose signal transducing function is associated with altered vascular permeability in VEGF-related diseases (International Patent Publication Nos. WO 97/40830 and WO 97/40831).

International Patent Publication No. WO 03066632 describes heterocyclic sulfonamide compounds with 5-HT6 receptor affinity. International Patent Publication No. WO 04046124 describes benzoxazinones as ligands for 5-HT1 receptors and their use in the treatment of CNS disorders. International Patent Publication No. WO 03022214 describes piperazine and homopiperazine compounds useful in the treatment of thrombosis and to inhibit ADP-mediated platelet aggregation. International Patent Publication No. WO 02066446 describes heterocyclic substituted cycloalkabecarboxamides as dopamine D3 receptor ligands. International Patent Publication No. WO 02032872 describes urea derivatives containing nitrogenous aromatic ring compounds as inhibitors of angiogenesis. U.S. Pat. No. 6,187,778 describes 4-aminopyrrolo[3,2-d]pyrimidines as neuropeptide Y receptor antagonists. International Patent Publication No. WO 9632391 describes pyrrolopyridines. U.S. Pat. No. 5,681,959 describes azaindoles. U.S. Pat. Nos. 5,178,997 and 5,389,509 describes high chloride tabular grain emulsions U.S. Pat. No. 5,053,408 describes heterocyclylhexitols as coronary vasodilators. International Patent Publication No. WO 04013139 describes 7-azaindole derivatives as dopamine D4 ligands and corticotrophin releasing hormone receptor antagonists. U.S. Patent Publication No. 2003220365 describes bicyclic heterocyclic compounds used for treating reperfusion injuries, inflammatory diseases, and autoimmune diseases. International Patent Publication No. WO 02016348 describes bicyclic derivatives for antiangiogenic and vascular permeability reducing effects for treating cancer, diabetes, psoriasis, arthritis, inflammation, and restenosis.

International Patent Publication No. WO 05062795 describes compounds and methods for development of Ret modulators. International Patent Publication No. WO 05051304 describes Akt kinase inhibitors.

International Patent Publication No. WO 05074642 describes substituted thiophene-2-carboxamide rho-associated kinase inhibitors useful for treating hypertension, restenosis, atherosclerosis, asthma, stroke, Alzheimer's disease, rheumatoid arthritis, cancer and diabetes. International Patent Publication No. WO 05074643 describes benzamide rho-associated coiled coil-forming protein kinase inhibitors for treatment of cardiovascular diseases, restenosis, atherosclerosis, asthma, stroke and multiple sclerosis. International Patent Publication No. WO05080394 describes 4-substituted piperidine derivative rho kinase inhibitors for treatment of injury or disease of the central nervous system, cancer and macular degeneration. International Patent Publication No. WO05103050 describes azaindoles useful as inhibitors of ROCK and other protein kinases. International Patent Publication No. WO0009162 describes rho kinase inhibitory agents for preventing or treating glaucoma.

### SUMMARY OF THE INVENTION

The present invention relates to compounds of Formula I: 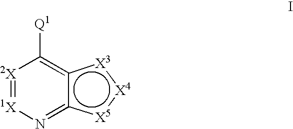
or a pharmaceutically acceptable salt thereof. The compounds of Formula I inhibit kinase enzymes and are useful for the treatment and/or prevention of hyperproliferative diseases such as cancer, inflammation, allergy, asthma, disease and conditions of the immune system, disease and conditions of the central nervous system, cardiovascular diseases, disease and conditions of the eye, dermatology, osteoporosis, diabetes, type-2 diabetes, multiple sclerosis, and viral infections.

Such cardiovascular diseases include hypertension, vasospasm, preterm labor, atherosclerosis, myocardial hypertrophy, erectile dysfunction, restenosis. Ocular diseases include glaucoma, diabetic retinopathy, choroidal neovascularization due to age-related macular degeneration, retinopathy of prematurity. Cancers include vascular smooth muscle cell hyperproliferation, bladder cancer, pancreatic cancer, testicular cancer, colon cancer, other hyperproliferative disorders. Cancer treatment includes limiting the toxicity of cytotoxics that act in S-phase, G2 or mitosis. Cancer treatment include limiting angiogenic processes or the formation of vascular hyperpermeability that lead to edema, ascites, effusions, exudates, and macromolecular extravasation and matrix deposition. Inflammatory diseases include endothelial dysfunction inflammation, arthritis, rheumatoid arthritis, CNS conditions and diseases include neurological diseases, neurodegenerative disorders, stroke, Alzheimer's disease. Disease and conditions of the immune system include autoimmune disorders, allograft rejection, and graft vs. host disease, AIDS, hyper-immune responses. Dermatological diseases include psoriasis, infantile hemangiomas. Viral infection treatment includes disrupting the virus life cycle by preventing virus replication.

### DETAILED DESCRIPTION OF THE INVENTION

The present invention relates to a compound of Formula I: 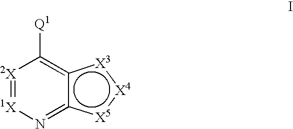
or a pharmaceutically acceptable salt thereof, wherein:

X¹ or X² are each independently N or —C(E¹)-;

X³, X⁴ and X⁵ are each independently N, O, S, —C(E^{1a})-, or ═C(E¹)-;

provided that
X³ is O or S when X⁴ and X⁵ are combined to equal —C(E^{1a})=C(E¹)-;
X⁵ is NH, O, or S when X³ and X⁴ are combined to equal —C(E^{1a})=C(E¹)-;
X⁵ is NH when X³ and X⁴ are combined to equal —N═C(E¹)-;
X⁵ is NH when X³ and X⁴ are combined to equal —C(E¹)=N—;

Q¹ is C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, heterobicycloC₅₋₁₀alkyl, spiroalkyl, or heterospiroalkyl; or -(Z¹)ₙ-(Y¹)ₘ—R¹; any of which is optionally substituted by one or more independent G¹ substituents;

E¹, E^{1a}, and G¹ are, in each instance, each independently equal to halo, —CF₃, —OCF₃, —OR², —NR²R³(R⁴)ⱼ₁, —C(═O)R², —CO₂R², —CONR²R³, —NO₂, —CN, —S(O)ⱼ,R², —SO₂NR²R³, —NR²C(═O)R³, —NR²C(═O)OR³, —NR²C(═O)NR³R⁴, —NR²S(O)ⱼ,R³, —C(═S)OR², —C(═O)SR², —NR²C(═NR³)NR⁴R⁵, —NR²C(═NR³)OR⁴, —NR²C(═NR³)SR⁴, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, oxo, —CF₃, —OCF₃, —OR²², —NR²²R³³(R^{22a})ⱼ₁ₐ, —C(O)R²², —CO₂R²², —C(═O)NR²²R³³, —NO₂, —CN, —S(═O)ⱼ₁ₐR²², —S₂NR²²R³³, —NR²²C(═O)R³³, —NR²²C(═O)OR³³, —NR²²C(═O)NR³³R^{22a}, —NR²²S(O)ⱼ₁ₐR²², —C(═S)OR²², —C(═O)SR²², —NR²²C(═NR³³)NR^{22a}R^{33a}, —NR²²C(═NR³³)OR^{22a}, —NR²²C(═NR³³)SR^{22a}, —C(═O)OR²², —OC(═O)NR²²R³³, —OC(═O)SR²², —SC(═O)OR²², or —SC(═O)NR²²R³³ substituents;

Z¹ is cycloC₃₋₈alkyl, heterocyclyl-C₀₋₁₀alkyl, aryl-C₀₋₁₀alkyl, hetaryl-C₀₋₁₀alkyl, heterobicycloC₅₋₁₀alkyl, spiroalkyl, or heterospiroalkyl, any of which is optionally substituted by one or more independent G¹ substituents;

Y¹ is —O—, —NR⁶, —S(O)ⱼ₂—, —CR^{6a}R^{7a}—, —N(C(O)OR⁶)—, —N(C(O)R⁶)—, —N(SO₂R⁶)—, —(CR^{6a}R^{7a})O—, —(CR^{6a}R^{7a})S—, —(CR^{6a}R^{7a})N(R⁶)—, —CR^{6a}(NR⁶)—, —(CR^{6a}R^{7a})N(C(O)R⁶), —(CR^{6a}R^{7a})N(C(O)OR⁶)—, —(CR^{6a}R^{7a})N(SO₂R⁶)—, —(CR^{6a})(NHR⁶)—, —(CR^{6a})(NHC(O)R⁶)—, —(CR^{6a})(NHSO₂R⁶)—, —(CR^{6a})(NHC(O)OR⁶)—, —(CR^{6a})(OC(O)R⁶)—, —(CR^{6a})(OC(O)NHR⁶)—, —(CR^{6a})═(CR^{6a})—, —C≡C—, —C(═NOR⁶)—, —C(O)—, —(CR^{6a})(OR⁶), —C(O)N(R⁶)—, —N(R⁶)C(O)—, —N(R⁶)S(O)—, —N(R⁶)S(O)₂— —OC(O)N(R⁶)—, N(R⁶)C(O)N(R^{6a})—, —NR⁶C(O)O—, —S(O)N(R⁶)—, —S(O)₂N(R⁶)—, —N(C(O)R⁶)S(O)—, —N(C(O)R⁶)S(O)₂—, —N(R⁶)S(O)N(R⁷)—, —N(R⁶)S(O)₂N(R⁷)—, —C(O)N(R⁶)C(O)—, —S(O)N(R⁷)C(O)—, —S(O)₂N(R⁶)C(O)—, —OS(O)N(R⁶)—, —OS(O)₂N(R⁶)—, —N(R⁶)S(O)O—, —N(R⁶)S(O)₂O—, —N(R⁶)S(O)C(O)—, —N(R⁶)S(O)₂C(O)—, —SON(C(O)R⁶)—, —SO₂N(C(O)R⁶)—, N(R⁶)SON(R⁷), —N(R⁶)SO₂N(R⁷)—, —C(O)O—, —N(R⁶)P(OR⁷)O—, —N(R⁶)P(OR⁷)—, —N(R⁶)P(O)(OR⁷)O—, —N(R⁶)P(O)(OR⁷)—, —N(C(O)R⁶)P(OR⁷)O—, —N(C(O)R⁶)P(OR⁷)—, —N(C(O)R⁶)P(O)(OR⁷)O—, —N(C(O)R⁶)P(OR⁷)—, —(CR^{6a}R^{7a})S(O)—, —(CR^{6a}R^{7a})S(O)₂—, —(CR^{6a}R^{7a})N(C(O)OR⁷)—, —(CR^{6a}R^{7a})N(C(O)R⁷)—, —(CR^{6a}R^{7a})N(SO₂R⁷)—, —(CR^{6a}R^{7a})C(═NOR⁷)—, —(CR^{6a}R^{7a})C(O)—, —(CR^{6a}R^{7a})(CR⁶aa)(OR⁷)—, —(CR^{6a}R^{7a})C(O)N(R⁷), —(CR^{6a}R^{7a})N(R⁶)C(O)—, —(CR^{6a}R^{7a})N(R⁷)S(O), —(CR^{6a}R^{7a})N(R⁷)S(O)₂—, —(CR^{6a}R^{7a})OC(O)N(R⁷)—, —(CR^{6a}R^{7a})N(R¹)C(O)N(R)—, —(CR^{6a}R^{7a})NR⁷C(O)O—, —(CR^{6a}R^{7a})S(O)N(R⁷), —(CR^{6a}R^{7a})S(O)₂N(R⁷)—, —(CR^{6a}R^{7a})N(C(O)R⁷)S(O)—, —(CR^{6a}R^{7a})N(C(O)R⁷)S(O)—, —(CR^{6a}R^{7a})N(R⁷)S(O)N(R⁸)—, —(CR^{6a}R^{7a})N(R⁷)S(O)₂N(R⁸)—, —(CR^{6a}R⁷³)C(O)N(R⁷)C(O)—, —(CR^{6a}R^{7a})S(O)N(R⁷)C(O)—, —(CR^{6a}R^{7a})S(O)₂N(R⁷)C(O)—, —(CR^{6a}R^{7a})OS(O)N(R⁷)—, —(CR^{6a}R^{7a})OS(O)₂N(R⁷)—, —(CR^{6a}R^{7a})N(R⁷)S(O)O—, —(CR^{6a}R^{7a})N(R⁷)S(O)₂O—, —(CR^{6a}R^{7a})N(R⁷)S(O)C(O)—, —(CR^{6a}R^{7a})N(R⁷)S(O)₂C(O)—, —(CR^{6a}R^{7a})SON(C(O)R⁷)—, —(CR^{6a}R^{7a})SO₂N(C(O)R⁷)—, —(CR^{6a}R^{7a})N(R⁷)SON(R⁸)—, —(CR^{6a}R^{7a})N(R⁷)SO₂N(R⁸)—, —(CR^{6a}R^{7a})C(O)O—, —(CR^{6a}R^{7a})N(R⁷)P(OR⁸)O—, —(CR^{6a}R^{7a})N(R⁷)P(OR⁸)—, —(CR^{6a}R^{7a})N(R⁷)P(O)(OR⁸)O—, —(CR^{6a}R^{7a})N(R⁷)P(O)(OR⁸)—, —(CR^{6a}R^{7a})N(C(O)R⁷)P(OR⁸)O—, —(CR^{6a}R^{7a})N(C(O)R⁷)P(OR⁸)—, —(CR^{6a}R^{7a})N(C(O)R⁷)P(O)(OR⁸)O—, or —(CR^{6a}R^{7a})N(C(O)R⁷)P(OR⁸)—;

R¹, R², R³, R⁴,R⁵, R⁶, R⁷, R⁸, R²²R^{22a}, R³³, and R^{33a} are, in each instance, each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹ substituents;

R^{6a}, R^{6aa}, and R^{7a} are, in each instance, each independently fluoro, trifluoromethyl, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G^{11a} substituents;

or in the case of —NR²R³(R⁴)ⱼ₁, —NR³R⁴, —NR⁴R⁵, NR^{2b}R^{3b}(R^{4b})_{j1b}, —NR^{3b}R^{4b}, —NR^{4b}R^{5b}, —NR⁹R¹⁰, —NR¹⁰R¹¹, —NR¹¹R¹², —NR²²R³³(R^{22a})ⱼ₁ₐ, —NR^{22a}R^{33a}, —NR³³R^{22a}, —NR⁶R¹, —NR⁷R¹, and —NR⁸R¹ then R² and R³, or R³ and R⁴, or R⁴ and R⁵, R^{2b} and R^{3b}, or R^{3b} and R^{4b}, or R^{4b} and R^{5b}, or R⁹ and R¹⁰, or R¹⁰ and R¹¹, or R¹¹ and R¹², or R²² and R³³, or R^{22a} and R^{33a}, or R³³ and R^{22a}, or R⁶ and R¹, or R⁷ and R¹, or R⁸ and R¹, respectively, are optionally taken together with the nitrogen atom to which they are attached to form a 3-10 membered saturated or unsaturated ring, wherein said ring is optionally substituted by one or more independent G . . . substituents and wherein said ring optionally includes one or more heteroatoms other than the nitrogen to which R² and R³, or R³ and R⁴, or R⁴ and R⁵, R^{2b} and R^{3b}, or R^{3b} and R^{4b}, or R^{4b} and R^{5b}, or R⁹ and R¹⁰, or R¹⁰ and R¹¹, or R¹¹ and R¹², or R²² and R³³, or R^{22a} and R^{33a}, or R³³ and R^{22a}, or R⁶ and R¹, or R⁷ and R¹, or R⁸ and R¹ are respectively attached;

or in the case of CR^{6a}R^{7a}, R^{6a} and R^{7a} can be taken together with the carbon to which they are attached to form a 3-10 membered saturated or unsaturated cycloalkyl or heterocycloalkyl ring, wherein said ring is optionally substituted by one or more independent G^{111a} substituents and wherein said ring optionally includes one or more heteroatoms;

G¹¹, G^{11a}, G¹¹¹, and G^{111a} are, in each instance, each independently halo, —CF₃, —OCF₃, —OR^{2b}, —NR^{2b}R^{3b}(R^{4b})_{j1b}, —C(═O)R^{2b}, —CO₂R^{2b}, CONR^{2b}R^{3b}, —NO₂, —CN, —S(O)_{j1b}R^{2b}, —SO₂NR^{2b}R^{3b}, N^{2b}C(═O)R^{3b}, —NR^{2b}C(═O)OR^{3b}, —NR^{2b}C(═O)NR^{3b}R^{4b}, —NR^{2b}S(O)_{j1b}R^{3b}, C(═S)OR^{2b}, C(═O)SR^{2b}, —NR^{2b}C(═NR^{3b})NR^{4b}R^{5b}, —NR^{2b}C(═NR^{3b})OR^{4b}, —NR^{2b}C(═NR^{3b})SR^{4b}, —OC(═O)OR^{2b}, —OC(═O)NR^{2b}R^{3b}, —OC(═O)SR^{2b}, —SC(═O)OR^{2b}, —SC(═O)NR^{2b}R^{3b}, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR⁹, —NR⁹R¹⁰, —C(O)R⁹, —CO₂R⁹, —CONR⁹R¹⁰, —NO₂, —CN, —S(O)ⱼ₂ₐR⁹, —SO₂NR⁹R¹⁰, —NR⁹C(═O)R¹⁰, —NR⁹C(═O)OR¹⁰, —NR⁹C(═O)NR¹¹R¹⁰, —NR⁹S(O)ⱼ₂ₐR¹⁰, —C(═S)OR⁹, —C(═O)SR⁹, —NR⁹C(═NR¹⁰)NR¹¹R¹², —NR⁹C(═NR¹⁰)OR¹¹, —NR⁹C(═NR¹⁰)SR¹¹, —OC(═O)OR⁹, —OC(═O)NR⁹R¹⁰, —OC(═O)SR⁹, —SC(═O)OR⁹, —P(O)OR⁹OR¹⁰, or —SC(═O)NR⁹R¹⁰ substituents;

R^{2b}, R^{3b}, R^{4b}, R^{5b}, R⁹, R¹⁰, R¹¹ and R¹² are, in each instance, each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;

or R^{2b}, R^{3b}, R^{4b}, R^{5b}, R⁹, R¹⁰, R¹¹ and R¹² are, in each instance, each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₁₀alkyl) substituents;

j₁, j₁ₐ, j_{1b}, j₂, j₂ₐ, n, and m are, in each instance, each independently 0, 1, 2, or 3.

In an aspect of the present invention, a compound is represented by Formula I, or a pharmaceutically acceptable salt thereof, wherein X¹ or X² are each —C(E¹)-; X³ and X⁴ are combined to equal —C(E^{1a})=C(E¹)-; X⁵ is NH; Q¹ is aryl-C₀₋₁₀alkyl optionally substituted by one or more independent G¹ substituents; and the other variables are as described above for Formula I.

In a second aspect of the present invention; a compound is represented by Formula I, or a pharmaceutically acceptable salt thereof, wherein X¹ or X² are each —C(E¹)-; X³ and X⁴ are combined to equal —C(E^{1a})=C(E¹)-; X⁵ is NH; Q¹ is heterocyclyl-C₀₋₁₀alkyl optionally substituted by one or more independent G¹ substituents and the other variables are as described above for Formula I.

In a third aspect of the present invention, a compound is represented by Formula I, or a pharmaceutically acceptable salt thereof, wherein X¹ or X² are each —C(E¹)-; X³ and X⁴ are combined to equal —C(E^{1a})=C(E¹)-; X⁵ is NH; Q¹ is hetaryl-C₀₋₁₀alkyl optionally substituted by one or more independent G¹ substituents and the other variables are as described above for Formula I.

The compounds of the present invention include any one of

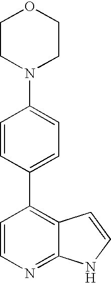

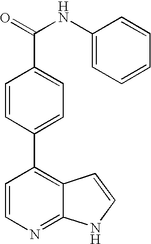

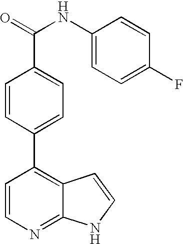

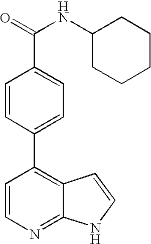

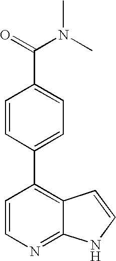

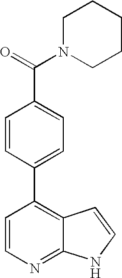

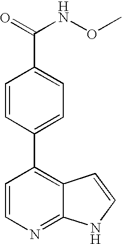

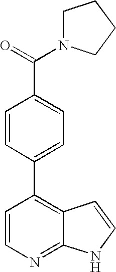

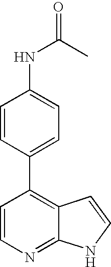

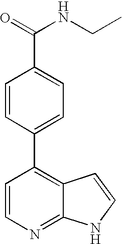

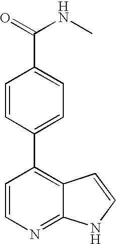

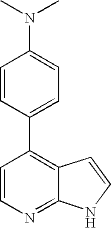

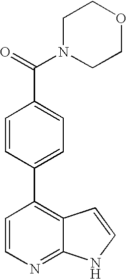

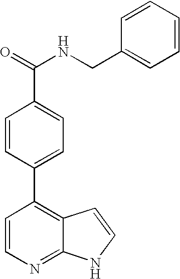

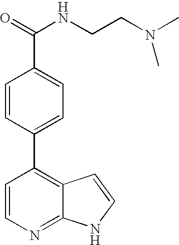

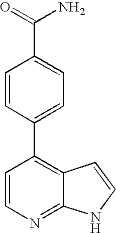

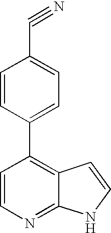

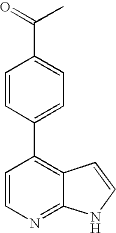

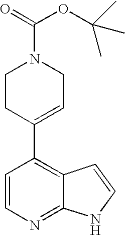

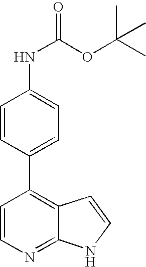

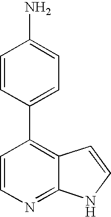

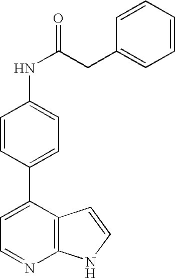

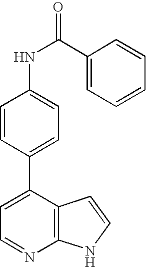

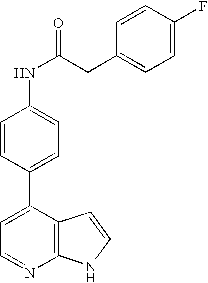

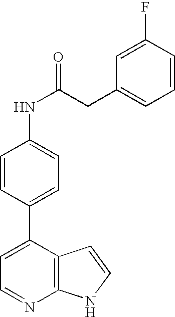

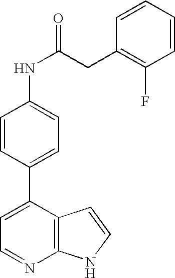

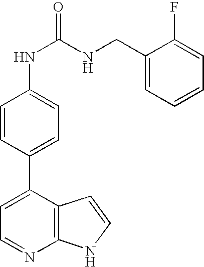

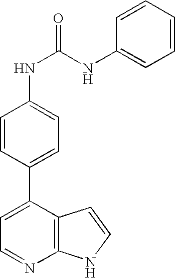

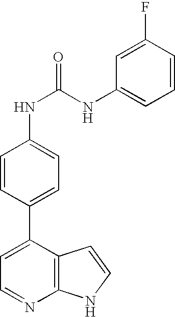

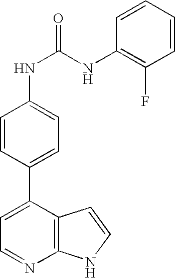

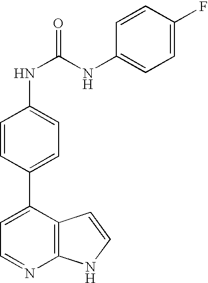

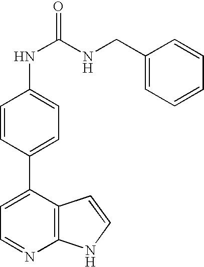

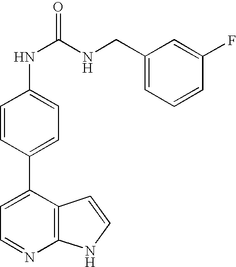

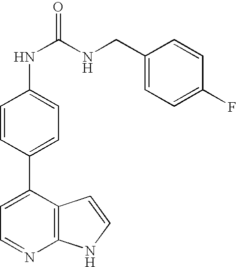

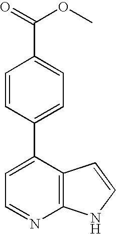

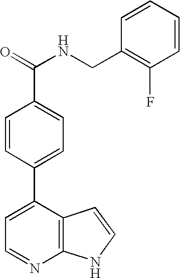

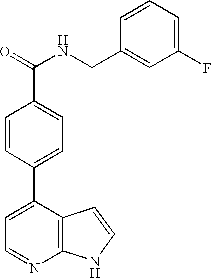

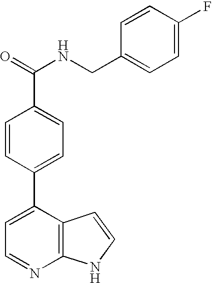

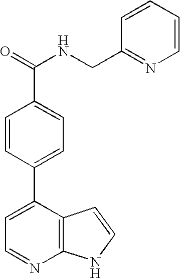

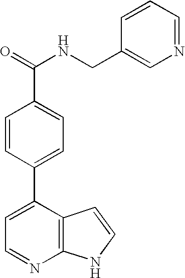

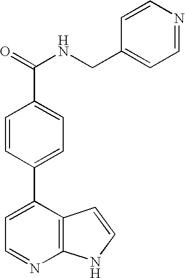

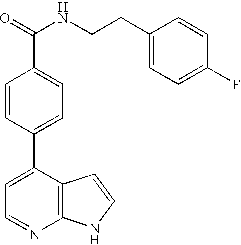

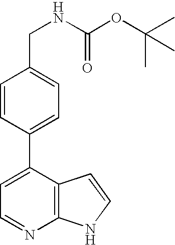

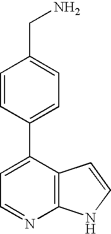

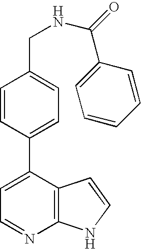

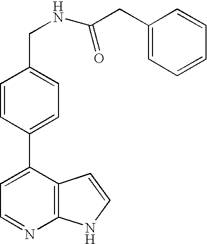

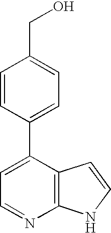

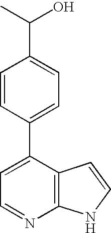

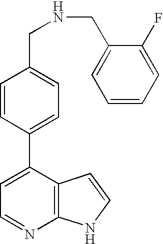

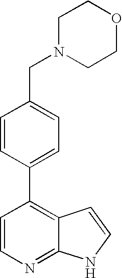

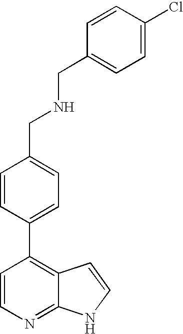

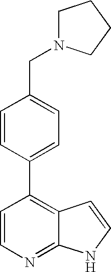

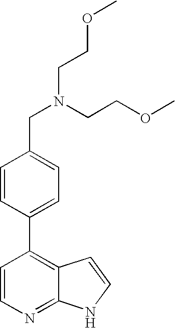

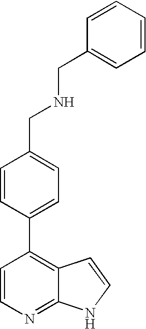

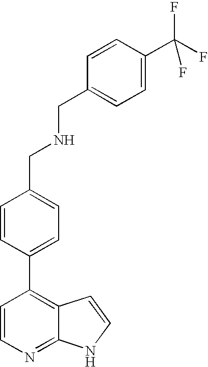

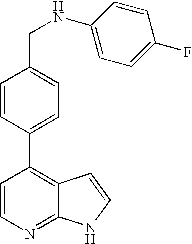

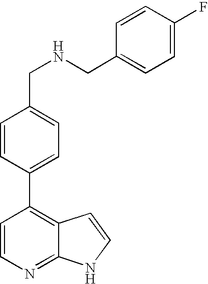

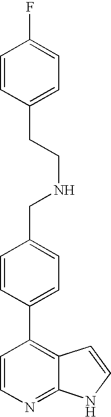

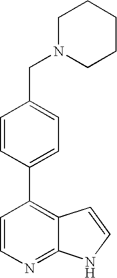

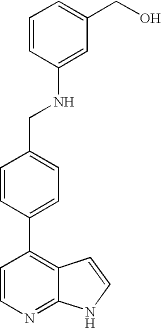

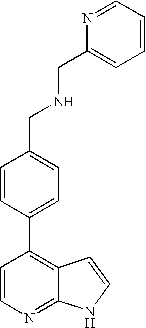

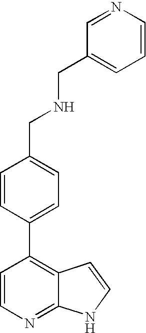

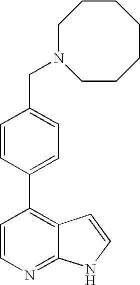

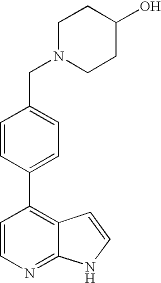

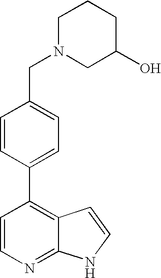

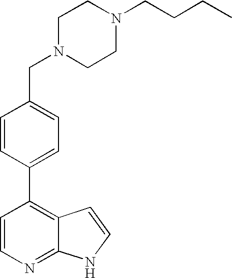

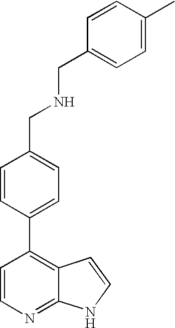

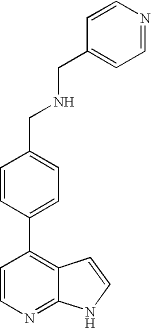

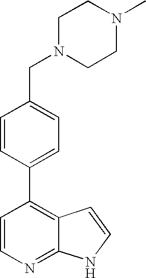

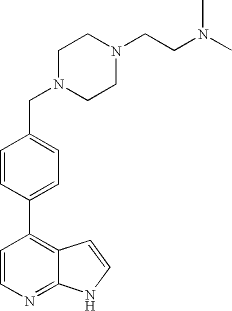

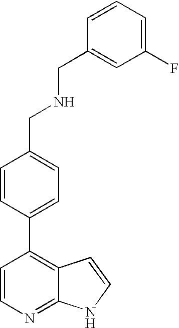

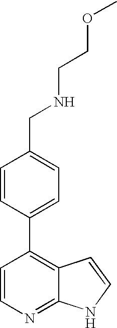

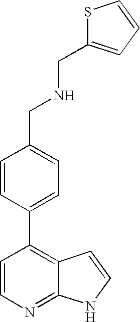

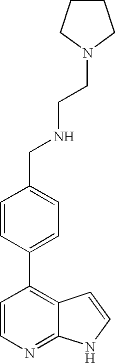

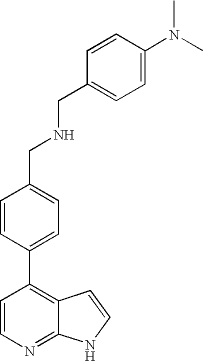

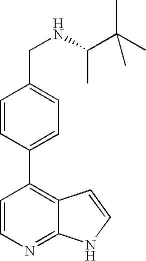

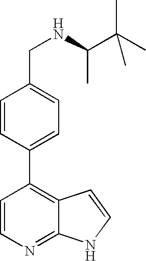

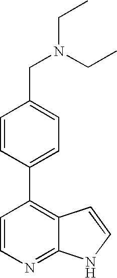

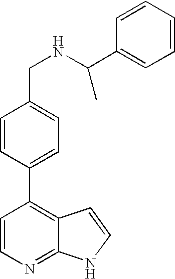

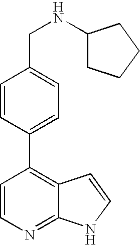

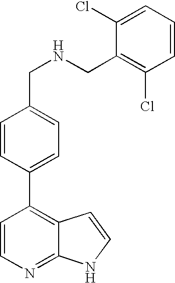

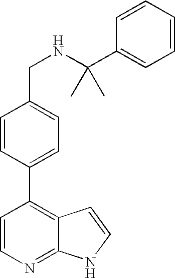

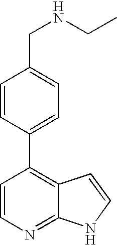

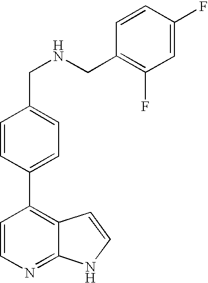

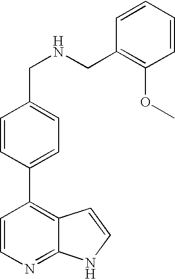

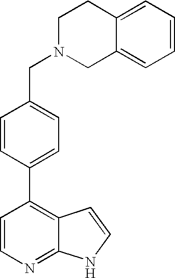

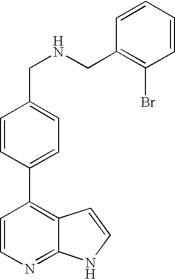

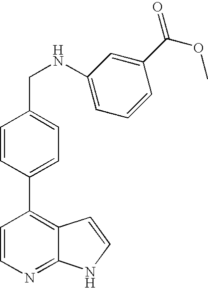

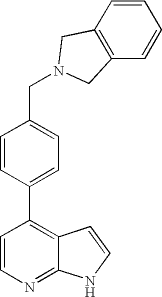

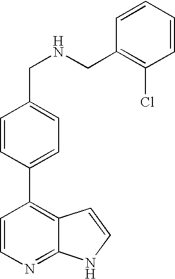

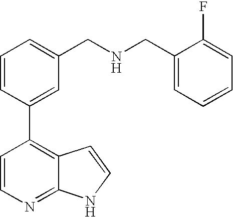

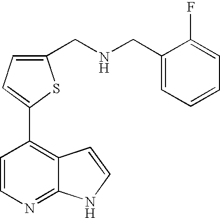

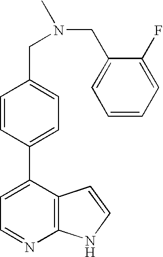

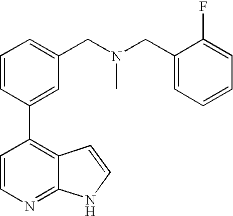

or a pharmaceutically acceptable salt thereof.

The compounds of this invention include 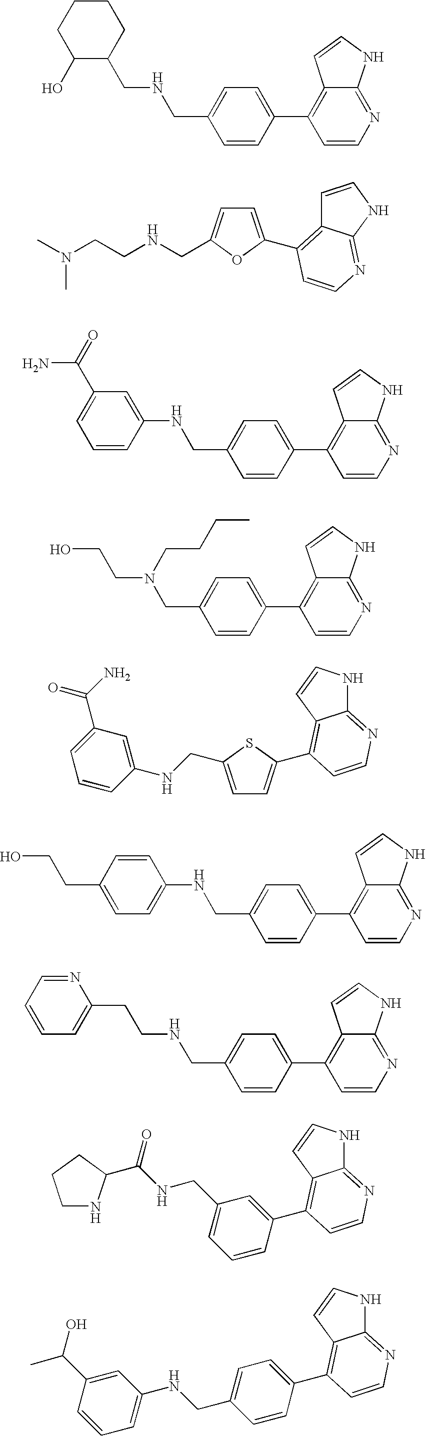 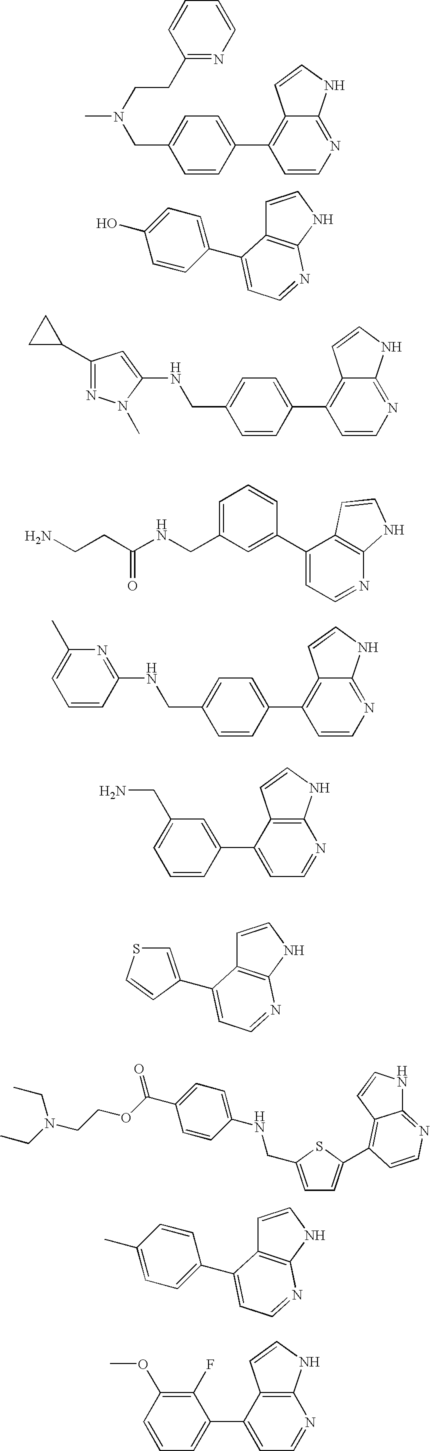 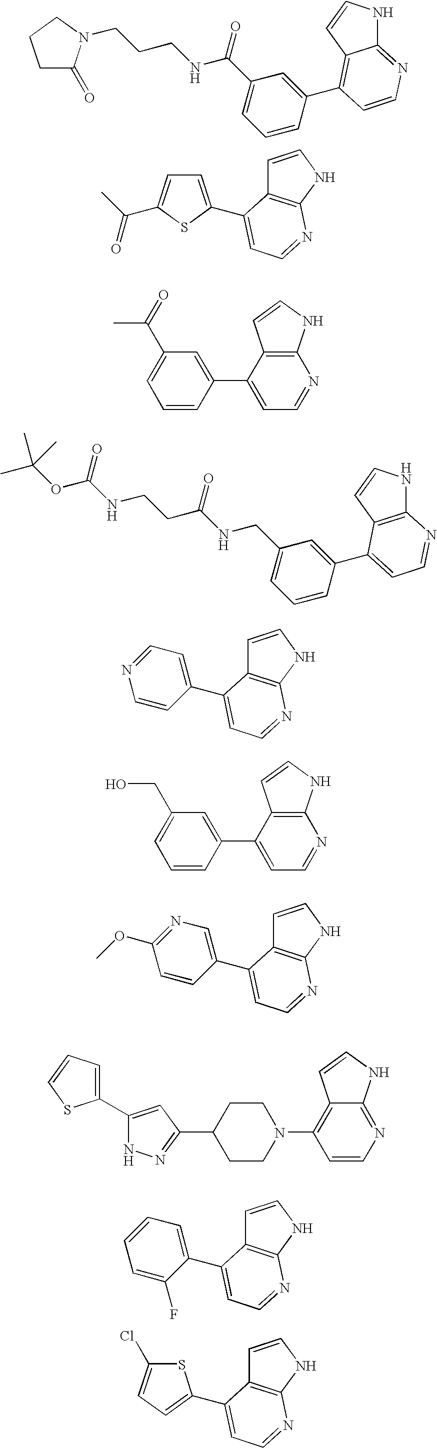 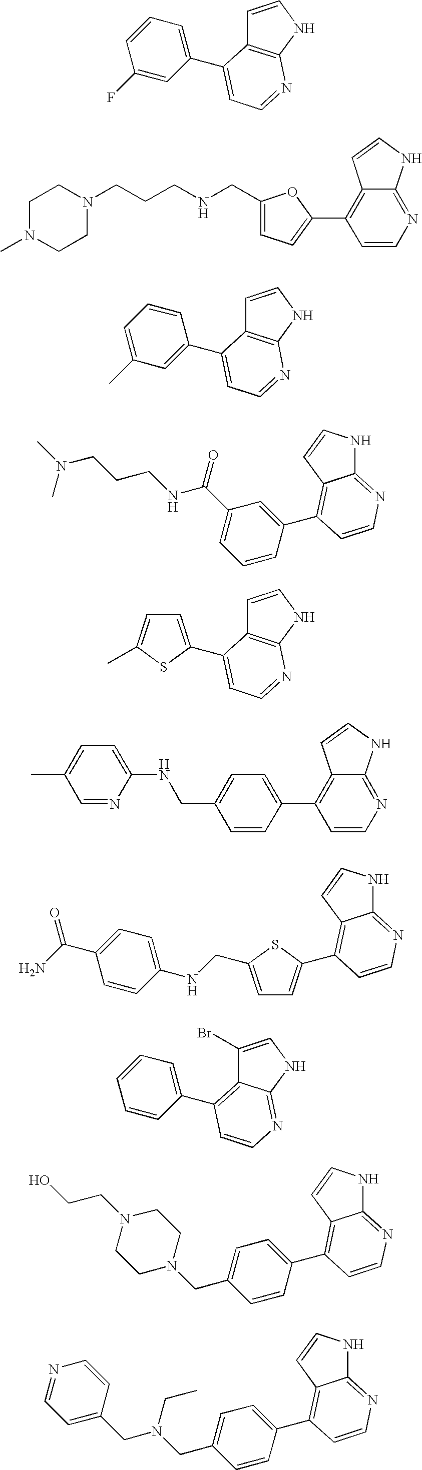 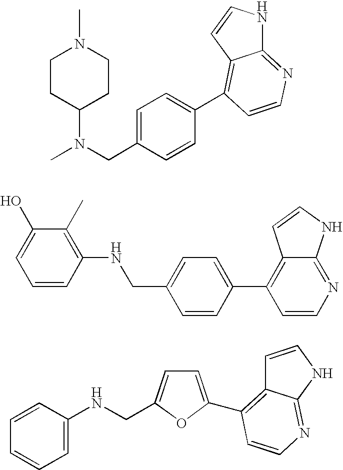
Or a pharmaceutically acceptable salt thereof.

The present invention includes a method of inhibiting protein kinase activity according to the present invention comprises administering a compound of Formula I, or a pharmaceutically acceptable salt thereof. The method includes wherein the protein kinase is ROCK. The method includes wherein the activity of the protein kinase affects hyperproliferative disorders. The method includes wherein the activity of the protein kinase influences angiogenesis, vascular permeability, immune response, cellular apoptosis, tumor growth, metastasis, or inflammation. The method includes wherein the activity of the protein kinase influences cardiovascular function including hypertension, ocular disorders and neuronal function. The method includes wherein the activity of the protein kinase influences cell migration or epithelial-mesenchymal transitions.

A method of the present invention of treating a patient having a condition that is mediated by protein kinase activity, comprises administering to the patient a therapeutically effective amount of a compound of Formula I, or a pharmaceutically acceptable salt thereof. The method includes wherein the protein kinase is ROCK. The method includes wherein the condition mediated by protein kinase activity is a hyperproliferative disorder. The method includes wherein the activity of the protein kinase influences angiogenesis, vascular permeability, immune response, cellular apoptosis, tumor growth, or inflammation. The method includes wherein the protein kinase is a protein serine/threonine kinase or a protein tyrosine kinase. The method includes wherein the condition mediated by protein kinase activity is one or more ulcers. The method includes wherein the ulcer or ulcers are caused by a bacterial or fungal infection; or the ulcer or ulcers are Mooren ulcers; or the ulcer or ulcers are a symptom of ulcerative colitis. The method includes wherein the condition mediated by protein kinase activity is Lyme disease, sepsis or infection by Herpes simplex, Herpes Zoster, human immunodeficiency virus, parapoxvirus, protozoa, or toxoplasmosis. The method includes wherein the condition mediated by protein kinase activity is von Hippel Lindau disease, pemphigoid, psoriasis, Paget's disease, or polycystic kidney disease. The method includes wherein the condition mediated by protein kinase activity is fibrosis, sarcoidosis, cirrhosis, thyroiditis, hyperviscosity syndrome, Osler-Weber-Rendu disease, chronic occlusive pulmonary disease, asthma, exudates, ascites, pleural effusions, pulmonary edema, cerebral edema or edema following bums, trauma, radiation, stroke, hypoxia, or ischemia. The method includes wherein the condition mediated by protein kinase activity is ovarian hyperstimulation syndrome, preeclampsia, menometrorrhagia, or endometriosis. The method includes wherein the condition mediated by protein kinase-activity is chronic inflammation, systemic lupus, glomerulonephritis, synovitis, inflammatory bowel disease, Crohn's disease, glomerulonephritis, rheumatoid arthritis and osteoarthritis, multiple sclerosis, or graft rejection.

The method includes wherein the condition mediated by protein kinase activity is sickle cell anemia. The method includes wherein the condition mediated by protein kinase activity is an ocular condition. The method includes wherein the ocular condition is ocular or macular edema, ocular neovascular disease, seleritis, radial keratotomy, uveitis, vitritis, myopia, optic pits, chronic retinal detachment, post-laser treatment complications, conjunctivitis, Stargardt's disease, Eales disease, retinopathy, or macular degeneration. The method includes wherein the condition mediated by protein kinase activity is a cardiovascular condition. The method includes wherein the condition mediated by protein kinase activity is atherosclerosis, restenosis, ischemia/reperfusion injury, vascular occlusion, venous malformation, or carotid obstructive disease. The method includes wherein the condition mediated by protein kinase activity is cancer. The method includes wherein the cancer is a solid tumor, a sarcoma, fibrosarcoma, osteoma, melanoma, retinoblastoma, a rhabdomyosarcoma, glioblastoma, neuroblastoma, teratocarcinoma, or metastases thereof, an hematopoietic malignancy, or malignant ascites. The method includes wherein the cancer is Kaposi's sarcoma, Hodgkin's disease, lymphoma, myeloma, or leukemia. Further, the method includes wherein the condition mediated by protein kinase activity is Crow-Fukase (POEMS) syndrome or a diabetic condition. The method includes wherein the diabetic condition is insulin-dependent diabetes mellitus glaucoma, diabetic retinopathy, or microangiopathy. The method also includes wherein the protein kinase activity is involved in T cell activation, B cell activation, mast cell degranulation, monocyte activation, signal transduction, apoptosis, the potentiation of an inflammatory response or a combination thereof.

The present invention includes the use of a compound of Formula I, or a pharmaceutically acceptable salt thereof, for the preparation of a pharmaceutical composition for the treatment of a disease that responds to an inhibition of the ROCK dependent cell proliferation.

The present invention includes the use of a compound of Formula I, or a pharmaceutically acceptable salt thereof, for the preparation of a pharmaceutical composition for the treatment of a disease that responds to an inhibition of the ROCK kinase.

The present invention includes a pharmaceutical composition comprising a therapeutically effective amount of a compound of Formula I, or a pharmaceutically acceptable salt thereof, and a pharmaceutically acceptable carrier. The invention includes a method of inhibiting protein kinase activity that comprises administering such pharmaceutical composition. The invention includes a method of treating a patient having a condition that is mediated by protein kinase activity by administering to the patient a therapeutically effective amount of such pharmaceutical composition.

The compounds of the present invention include:
4-(4-Morpholin-4-yl-phenyl)-1H-pyrrolo[2,3-b]pyridine;
N-Phenyl-4-(1H-pyrrolo[2,3-b]pyridin-4-yl)-benzamide;
N-(4-Fluoro-phenyl)-4-(1H-pyrrolo[2,3-b]pyridin-4-yl)-benzamide;
N-Cyclohexyl-4-(1H-pyrrolo[2,3-b]pyridin-4-yl)-benzamide;
N,N-Dimethyl-4-(1H-pyrrolo[2,3-b]pyridin-4-yl)-benzamide;
Piperidin-1-yl-[4-(1H-pyrrolo[2,3-b]pyridin-4-yl)-phenyl]-methanone;
N-Methoxy-4-(1H-pyrrolo[2,3-b]pyridin-4-yl)-benzamide;
Pyrrolidin-1-yl-[4-(1H-pyrrolo[2,3-b]pyridin-4-yl)-phenyl]-methanone;
N-[4-(1H-pyrrolo[2,3-b]pyridin-4-yl)-phenyl]-acetamide;
N-Ethyl-4-(1H-pyrrolo[2,3-b]pyridin-4-yl)-benzamide;
N-Methyl-4-(1H-pyrrolo[2,3-b]pyridin-4-yl)-benzamide;
Dimethyl-[4-(1H-pyrrolo[2,3-b]pyridin-4-yl)-phenyl]-amine;
Morpholin-4-yl-[4-(1H-pyrrolo[2,3-b]pyridin-4-yl)-phenyl]-methanone;
N-Benzyl-4-(1H-pyrrolo[2,3-b]pyridin-4-yl)-benzamide;
N-(2-Dimethylamino-ethyl)-4-(1H-pyrrolo[2,3-b]pyridin-4-yl)-benzamide;
4-(1H-Pyrrolo[2,3-b]pyridin-4-yl)-benzamide;
4-(1H-Pyrrolo[2,3-b]pyridin-4-yl)-benzonitrile;
1-[4-(1H-Pyrrolo[2,3-b]pyridin-4-yl)-phenyl]-ethanone;
4-(1H-Pyrrolo[2,3-b]pyridin-4-yl)-3,6-dihydro-2H-pyridine-1-carboxylic acid tert-butyl ester;
[4-(1H-Pyrrolo[2,3-b]pyridin-4-yl)-phenyl]-carbamic acid tert-butyl ester;
4-(1H-Pyrrolo[2,3-b]pyridin-4-yl)-phenylamine;
2-Phenyl-N-[4-(1H-pyrrolo[2,3-b]pyridin-4-yl)-phenyl]-acetamide;
N-[4-(1H-Pyrrolo[2,3-b]pyridin-4-yl)-phenyl]-benzamide;
2-(4-Fluoro-phenyl)-N-[4-(1H-pyrrolo[2,3-b]pyridin-4-yl)-phenyl]-acetamide;
2-(3-Fluoro-phenyl)-N-[4-(1H-pyrrolo[2,3-b]pyridin-4-yl)-phenyl]-acetamide;
2-(2-Fluoro-phenyl)-N-[4-(1H-pyrrolo[2,3-b]pyridin-4-yl)-phenyl]-acetamide;
1-(2-Fluoro-benzyl)-3-[4-(1H-pyrrolo[2,3-b]pyridin-4-yl)-phenyl]-urea;
1-Phenyl-3-[4-(1H-pyrrolo[2,3-b]pyridin-4-yl)-phenyl]-urea;
1-(3-Fluoro-phenyl)-3-[4-(1H-pyrrolo[2,3-b]pyridin-4-yl)-phenyl]-urea;
1-(2-Fluoro-phenyl)-3-[4-(1H-pyrrolo[2,3-b]pyridin-4-yl)-phenyl]-urea;
1-(4-Fluoro-phenyl)-3-[4-(1H-pyrrolo[2,3-b]pyridin-4-yl)-phenyl]-urea;
1-Benzyl-3-[4-(H-pyrrolo[2,3-b]pyridin-4-yl)-phenyl]-urea;
1-(3-Fluoro-benzyl)-3-[4-(1H-pyrrolo[2,3-b]pyridin-4-yl)-phenyl]-urea;
1-(4-Fluoro-benzyl)-3-[4-(1H-pyrrolo[2,3-b]pyridin-4-yl)-phenyl]-urea;
4-(1H-pyrrolo[2,3-b]pyridin-4-yl)-benzoic acid methyl ester;
N-(2-Fluoro-benzyl)-4-(1H-pyrrolo[2,3-b]pyridin-4-yl)-benzamide;
N-(3-Fluoro-benzyl)-4-(1H-pyrrolo[2,3-b]pyridin-4-yl)-benzamide;
N-(4-Fluoro-benzyl)-4-(1H-pyrrolo[2,3-b]pyridin-4-yl)-benzamide;
N-Pyridin-2-ylmethyl-4-(1H-pyrrolo[2,3-b]pyridin-4-yl)-benzamide;
N-Pyridin-3-ylmethyl-4-(1H-pyrrolo[2,3-b]pyridin-4-yl)-benzamide;
N-Pyridin-4-ylmethyl-4-(1H-pyrrolo[2,3-b]pyridin-4-yl)-benzamide;
N-[2-(4-Fluoro-phenyl)-ethyl]-4-(1H-pyrrolo[2,3-b]pyridin-4-yl)-benzamide;
[4-(1H-pyrrolo[2,3-b]pyridin-4-yl)-benzyl]-carbamic acid tert-butyl ester;
4-(1H-pyrrolo[2,3-b]pyridin-4-yl)-benzylamine;
N-[4-(1H-pyrrolo[2,3-b]pyridin-4-yl)-benzyl]-benzamide;
2-Phenyl-N-[4-(1H-pyrrolo[2,3-b]pyridin-4-yl)-benzyl]-acetamide;
[4-(1H-pyrrolo[2,3-b]pyridin-4-yl)-phenyl]-methanol;
1-[4-(1H-pyrrolo[2,3-b]pyridin-4-yl)-phenyl]-ethanol;
(2-Fluoro-benzyl)-[4-(1H-pyrrolo[2,3-b]pyridin-4-yl)-benzyl]-amine;
4-(4-Morpholin-4-ylmethyl-phenyl)-1H-pyrrolo[2,3-b]pyridine;
(4-Chloro-benzyl)-[4-(1H-pyrrolo[2,3-b]pyridin-4-yl)-benzyl]-amine;
4-(4-Pyrrolidin-1-ylmethyl-phenyl)-1H-pyrrolo[2,3-b]pyridine;
Bis-(2-methoxy-ethyl)-[4-(1H-pyrrolo[2,3-b]pyridin-4-yl)-benzyl]-amine;
Benzyl-[4-(1H-pyrrolo[2,3-b]pyridin-4-yl)-benzyl]-amine;
[4-(1H-pyrrolo[2,3-b]pyridin-4-yl)-benzyl]-(4-trifluoromethyl-benzyl)-amine;
(4-Fluoro-phenyl)-[4-(1H-pyrrolo[2,3-b]pyridin-4-yl)-benzyl]-amine; (4-Fluoro-benzyl)-[4-(1H-pyrrolo[2,3-b]pyridin-4-yl)-benzyl]-amine;
[2-(4-Fluoro-phenyl)-ethyl]-[4-(1H-pyrrolo[2,3-b]pyridin-4-yl)-benzyl]-amine;
4-(4-Piperidin-1-ylmethyl-phenyl)-1H-pyrrolo[2,3-b]pyridine;
{3-[4-(1H-pyrrolo[2,3-b]pyridin-4-yl)-benzylamino]-phenyl}-methanol;
Pyridin-2-ylmethyl-[4-(1H-pyrrolo[2,3-b]pyridin-4-yl)-benzyl]-amine;
Pyridin-3-ylmethyl-[4-(1H-pyrrolo[2,3-b]pyridin-4-yl)-benzyl]-amine;
4-(4-Azocan-1-ylmethyl-phenyl)-1H-pyrrolo[2,3-b]pyridine;
1-[4-(1H-pyrrolo[2,3-b]pyridin-4-yl)-benzyl]-piperidin-4-ol;
1-[4-(1H-pyrrolo[2,3-b]pyridin-4-yl)-benzyl]-piperidin-3-ol;
4-[4-(4-Butyl-piperazin-1-ylmethyl)-phenyl]-1H-pyrrolo[2,3-b]pyridine;
(4-Methyl-benzyl)-[4-(1H-pyrrolo[2,3-b]pyridin-4-yl)-benzyl]-amine;
Pyridin-4-ylmethyl-[4-(1H-pyrrolo[2,3-b]pyridin-4-yl)-benzyl]-amine;
4-[4-(4-Methyl-piperazin-1-ylmethyl)-phenyl]-1H-pyrrolo[2,3-b]pyridine;
Dimethyl-(2-{4-[4-(1H-pyrrolo[2,3-b]pyridin-4-yl)-benzyl]-piperazin-1-yl}-ethyl)-amine;
(3-Fluoro-benzyl)-[4-(1H-pyrrolo[2,3-b]pyridin-4-yl)-benzyl]-amine;
(2-Methoxy-ethyl)-[4-(1H-pyrrolo[2,3-b]pyridin-4-yl)-benzyl]-amine;
[4-(1H-pyrrolo[2,3-b]pyridin-4-yl)-benzyl]-thiophen-2-ylmethyl-amine;
(2-Pyrrolidin-1-yl-ethyl)-[4-(1H-pyrrolo[2,3-b]pyridin-4-yl)-benzyl]-amine;
Dimethyl-(4-{[4-(1H-pyrrolo[2,3-b]pyridin-4-yl)-benzylamino]-methyl}-phenyl)-amine;
(S)-[4-(1H-pyrrolo[2,3-b]pyridin-4-yl)-benzyl]-(1,2,2-trimethyl-propyl)-amine;
(R)-[4-(1H-pyrrolo[2,3-b]pyridin-4-yl)-benzyl]-(1,2,2-trimethyl-propyl)-amine;
Diethyl-[4-(1H-pyrrolo[2,3-b]pyridin-4-yl)-benzyl]-amine;
(1-Phenyl-ethyl)-[4-(1H-pyrrolo[2,3-b]pyridin-4-yl)-benzyl]-amine;
Cyclopentyl-[4-(1H-pyrrolo[2,3-b]pyridin-4-yl)-benzyl]-amine;
(2,6-Dichloro-benzyl)-[4-(1H-pyrrolo[2,3-b]pyridin-4-yl)-benzyl]-amine;
(1-Methyl-1-phenyl-ethyl)-[4-(1H-pyrrolo[2,3-b]pyridin-4-yl)-benzyl]-amine;
Ethyl-[4-(1H-pyrrolo[2,3-b]pyridin-4-yl)-benzyl]-amine;
(2,4-Difluoro-benzyl)-[4-(1H-pyrrolo[2,3-b]pyridin-4-yl)-benzyl]-amine;
(2-Methoxy-benzyl)-[4-(1H-pyrrolo[2,3-b]pyridin-4-yl)-benzyl]-amine;
2-[4-(1H-pyrrolo[2,3-b]pyridin-4-yl)-benzyl]-1,2,3,4-tetrahydro-isoquinoline;
(2-Bromo-benzyl)-[4-(1H-pyrrolo[2,3-b]pyridin-4-yl)-benzyl]-amine;
3-[4-(1H-pyrrolo[2,3-b]pyridin-4-yl)-benzylamino]-benzoic acid methyl ester;
4-[4-(1,3-Dihydro-isoindol-2-ylmethyl)-phenyl]-1H-pyrrolo[2,3-b]pyridine;
(2-Chloro-benzyl)-[4-(1H-pyrrolo[2,3-b]pyridin-4-yl)-benzyl]-amine;
(2-Fluoro-benzyl)-[3-(1H-pyrrolo[2,3-b]pyridin-4-yl)-benzyl]-amine;
(2-Fluoro-benzyl)-[5-(1H-pyrrolo[2,3-b]pyridin-4-yl)-thiophen-2-ylmethyl]-amine;
(2-Fluoro-benzyl)-methyl-[4-(1H-pyrrolo[2,3-b]pyridin-4-yl)-benzyl]-amine;
(2-Fluoro-benzyl)-methyl-[3-(1H-pyrrolo[2,3-b]pyridin-4-yl)-benzyl]-amine;
2-{[4-(1H-pyrrolo[2,3-b]pyridin-4-yl)-benzylamino]-methyl}-cyclohexanol;
N,N-Dimethyl-N′-[5-(1H-pyrrolo[2,3-b]pyridin-4-yl)-furan-2-ylmethyl]-ethane-1,2-diamine;
3-[4-(1H-Pyrrolo[2,3-]pyridin-4-yl)-benzylamino]-benzamide;
2-{Butyl-[4-(1H-pyrrolo[2,3-b]pyridin-4-yl)-benzyl]-amino}-ethanol;
3-{[5-(1H-Pyrrolo[2,3-b]pyridin-4-yl)-thiophen-2-ylmethyl]-amino}-benzamide;
2-{4-[4-(1H-pyrrolo[2,3-b]pyridin-4-yl)-benzylamino]-phenyl-ethanol;
(2-Pyridin-2-yl-ethyl)-[4-(1H-pyrrolo[2,3-b]pyridin-4-yl)-benzyl]-amine;
Pyrrolidine-2-carboxylic acid 3-(1H-pyrrolo[2,3-b]pyridin-4-yl)-benzylamide;
1-{3-[4-(1H-Pyrrolo[2,3-b]pyridin-4-yl)-benzylamino]-phenyl-ethanol;
4-(1H-Pyrrolo[2,3-b]pyridin-4-yl)-phenol;
Methyl-(2-pyridin-2-yl-ethyl)-[4-(1H-pyrrolo[2,3-b]pyridin-4-yl)-benzyl]-amine;
(5-Cyclopropyl-2-methyl-2H-pyrazol-3-yl)-[4-(1H-pyrrolo[2,3-b]pyridin-4-yl)-benzyl]-amine;
(6-Methyl-pyridin-2-yl)-[4-(1H-pyrrolo[2,3-b]pyridin-4-yl)-benzyl]-amine;
3-Amino-N-[3-(1H-pyrrolo[2,3-b]pyridin-4-yl)-benzyl]-propionamide;
3-(1H-Pyrrolo[2,3-b]pyridin-4-yl)-benzylamine;
4-Thiophen-3-yl-1H-pyrrolo[2,3-b]pyridine;
4-{[5-(1H-Pyrrolo[2,3-b]pyridin-4-yl)-thiophen-2-ylmethyl]-amino}-benzoic acid 2-diethylamino-ethyl ester;
4-p-Tolyl-1H-pyrrolo[2,3-b]pyridine;
N-[3-(2-Oxo-pyrrolidin-1-yl)-propyl]-3-(1H-pyrrolo[2,3-b]pyridin-4-yl)-benzamide;
4-(2-Fluoro-3-methoxy-phenyl)-1H-pyrrolo[2,3-b]pyridine;
1-[5-(1H-pyrrolo[2,3-b]pyridin-4-yl)-thiophen-2-yl]-ethanone;
{2-[3-(1H-pyrrolo[2,3-b]pyridin-4-yl)-benzylcarbamoyl]-ethyl}-carbamic acid tert-butyl ester;
1-[3-(1H-Pyrrolo[2,3-b]pyridin-4-yl)-phenyl]-ethanone;
4-Pyridin-4-yl-1H-pyrrolo[2,3-b]pyridine;
[3-(1H-pyrrolo[2,3-b]pyridin-4-yl)-phenyl]-methanol;
4-(6-Methoxy-pyridin-3-yl)-1H-pyrrolo[2,3-b]pyridine;
4-[4-(5-Thiophen-2-yl-1H-pyrazol-3-yl)-piperidin-1-yl]-1H-pyrrolo[2,3-b]pyridine;
4-(2-Fluoro-phenyl)-1H-pyrrolo[2,3-b]pyridine;
4-(5-Chloro-thiophen-2-yl)-1H-pyrrolo[2,3-b]pyridine;
4-(3-Fluoro-phenyl)-1H-pyrrolo[2,3-b]pyridine;
[3-(4-Methyl-piperazin-1-yl)-propyl]-[5-(1H-pyrrolo[2,3-b]pyridin-4-yl)-furan-2-ylmethyl]-amine;
4-m-Tolyl-1H-pyrrolo[2,3-b]pyridine;
N-(3-Dimethylamino-propyl)-3-(1H-pyrrolo[2,3-b]pyridin-4-yl)-benzamide;
4-(5-Methyl-thiophen-2-yl)-1H-pyrrolo[2,3-b]pyridine;
(5-Methyl-pyridin-2-yl)-[4-(1H-pyrrolo[2,3-b]pyridin-4-yl)-benzyl]-amine;
4-{[5-(1H-pyrrolo[2,3-b]pyridin-4-yl)-thiophen-2-ylmethyl]-amino}-benzamide;
3-Bromo-4-phenyl-1H-pyrrolo[2,3-b]pyridine; 2-{4-[4-(1H-Pyrrolo[2,3-b]pyridin-4-yl)-benzyl]-piperazin-1-yl}-ethanol;
Ethyl-pyridin-4-ylmethyl-[4-(1H-pyrrolo[2,3-b]pyridin-4-yl)-benzyl]-amine;
Methyl-(1-methyl-piperidin-4-yl)-[4-(1H-pyrrolo[2,3-b]pyridin-4-yl)-benzyl]-amine;
2-Methyl-3-[4-(1H-pyrrolo[2,3-b]pyridin-4-yl)-benzylamino]-phenol;
Phenyl-[5-(1H-pyrrolo[2,3-b]pyridin-4-yl)-furan-2-ylmethyl]-amine;
1-[4-(3-Bromo-1H-pyrrolo[2,3-b]pyridin-4-yl)-phenyl]-ethanone;
(5-Ethyl-[1,3,4]thiadiazol-2-yl)-[3-(1H-pyrrolo[2,3-b]pyridin-4-yl)-benzyl]-amine;
1-(4-Naphthalen-2-yl-1H-pyrrolo[2,3-b]pyridin-3-yl)-ethanone;
2-{4-[3-(1H-Pyrrolo[2,3-b]pyridin-4-yl)-benzyl]-piperazin-1-yl}-ethanol;
2-{[3-(1H-Pyrrolo[2,3-b]pyridin-4-yl)-benzylamino]-methyl}-cyclohexanol;
(1H-Benzotriazol-5-yl)-[4-(1H-pyrrolo[2,3-b]pyridin-4-yl)-benzyl]-amine;
2-{4-[3-(1H-Pyrrolo[2,3-b]pyridin-4-yl)-benzylamino]-phenyl}-ethanol;
4-[3-(1H-Pyrrolo[2,3-b]pyridin-4-yl)-benzylamino]-benzamide;
(5-Cyclopropyl-2-methyl-2H-pyrazol-3-yl)-[3-(1H-pyrrolo[2,3-b]pyridin-4-yl)-benzyl]-amine;
(6-Methyl-pyridin-2-yl)-[3-(1H-pyrrolo[2,3-b]pyridin-4-yl)-benzyl]-amine;
1-[4-(3-Chloro-1H-pyrrolo[2,3-b]pyridin-4-yl)-phenyl]-ethanone;
4-Benzo[1,3]dioxol-5-yl-3-bromo-1H-pyrrolo[2,3-b]pyridine;
N-(2,3-Dihydroxy-propyl)-3-(1H-pyrrolo[2,3-b]pyridin-4-yl)-benzamide;
N-Carbamoylmethyl-3-(1H-pyrrolo[2,3-b]pyridin-4-yl)-benzamide;
Isoquinolin-5-yl-[5-(1H-pyrrolo[2,3-b]pyridin-4-yl)-thiophen-2-ylmethyl]-amine;
3-[3-(1H-Pyrrolo[2,3-b]pyridin-4-yl)-benzylamino]-benzamide;
4-Benzo[1,3]dioxol-5-yl-3-chloro-1H-pyrrolo[2,3-b]pyridine;
3-Bromo-4-(4-vinyl-phenyl)-1H-pyrrolo[2,3-b]pyridine;
(3-[3-(1H-Pyrrolo[2,3-b]pyridin-4-yl)-benzylamino]-phenyl}-methanol;
(E)-4-[4-(3-Acetyl-1H-pyrrolo[2,3-b]pyridin-4-yl)-phenyl]-but-3-en-2-one;
3-[4-(1H-Pyrrolo[2,3-b]pyridin-4-yl)-benzylamino]-benzoic acid; 3-Chloro-4-phenyl-1H-pyrrolo[2,3-b]pyridine;
1-[4-(4-Acetyl-phenyl)-1H-pyrrolo[2,3-b]pyridin-3-yl]-ethanone;
1-(4-Phenyl-1H-pyrrolo[2,3-b]pyridin-3-yl)-ethanone;
1-[4-(3-Fluoro-phenyl)-1H-pyrrolo[2,3-b]pyridin-3-yl]-ethanone;
4-Biphenyl-4-yl-3-bromo-1H-pyrrolo[2,3-b]pyridine;
4-Thiophen-2-yl-1H-pyrrolo[2,3-b]pyridine;
N-[2-(1H-Imidazol-4-yl)-ethyl]-3-(1H-pyrrolo[2,3-b]pyridin-4-yl)-benzamide;
4-(4-Methanesulfonyl-phenyl)-1H-pyrrolo[2,3-b]pyridine;
4-(3,5-Difluoro-phenyl)-1H-pyrrolo[2,3-b]pyridine;
4-(6-Methoxy-pyridin-2-yl)-1H-pyrrolo[2,3-b]pyridine;
4-(2-Chloro-phenyl)-1H-pyrrolo[2,3-b]pyridine;
4-(3,4-Dimethoxy-phenyl)-1H-pyrrolo[2,3-b]pyridine;
4-(2,3-Difluoro-phenyl)-1H-pyrrolo[2,3-b]pyridine;
5-(1H-pyrrolo[2,3-b]pyridin-4-yl)-furan-2-carbaldehyde;
N,N-Dimethyl-N′-[5-(1H-pyrrolo[2,3-b]pyridin-4-yl)-furan-2-ylmethyl]-benzene-1,4-diamine;
N-(2-Dimethylamino-ethyl)-3-(1H-pyrrolo[2,3-b]pyridin-4-yl)-benzamide;
1-{3-[3-(1H-Pyrrolo[2,3-b]pyridin-4-yl)-benzylamino]-phenyl}-ethanol;
(1-Phenyl-ethyl)-[3-(1H-pyrrolo[2,3-b]pyridin-4-yl)-benzyl]-amine; and
1-[3-(1H-Pyrrolo[2,3-b]pyridin-4-yl)-benzyl]-piperidine-3-carboxylic acid amide.

Unless otherwise stated, the connections of compound name moieties are at the rightmost recited moiety. That is, the substituent name starts with a terminal moiety, continues with any bridging moieties, and ends with the connecting moiety. For example, hetarylthioC₁₋₄alkyl has a heteroaryl group connected through a thio sulfur to a C₁₋₄ alkyl that connects to the chemical species bearing the substituent.

In all of the above circumstances forbidden or unstable valences, such as but not limited to N-halogen or oxygen-oxygen bonds, are excluded.

As used herein—unless specifically identified as ROCK1 or ROCK2—the term “ROCK” will mean one of, or both of, the ROCK1 and ROCK2 isoforms.

As used herein, for example, “C₀₋₄alkyl” is used to mean an alkyl having 0-4 carbons—that is, 0, 1, 2, 3, or 4 carbons in a straight or branched configuration. An alkyl having no carbon is hydrogen when the alkyl is a terminal group. An alkyl having no carbon is a direct bond when the alkyl is a bridging (connecting) group. Further, C₀alkyl includes being a substituted bond—that is, for example, —X—Y-Z is —C(O)—C₂₋₄alkyl when X is C₀alkyl, Y is C₀alkyl, and Z is —C(O)—C₂₋₄alkyl.

In all embodiments of this invention, the term “alkyl” includes both branched and straight chain alkyl groups. Typical alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl, and the like.

The term “halo” refers to fluoro, chloro, bromo, or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl, and the like.

The term “acyl” refers to the structure —C(═O)—R, in which R is a general substituent variable such as, for example R¹ described above. Examples include, but are not limited to, (bi)(cyclo)alkylketo, (cyclo)alkenylketo, alkynylketo, arylketo, hetarylketo, heterocyclylketo, heterobicycloalkylketo, spiroalkylketo.

Unless otherwise specified, the term “cycloalkyl” refers to a 3-8 carbon cyclic aliphatic ring structure, optionally substituted with for example, alkyl, hydroxy, oxo, and halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl, and the like.

The term “bicycloalkyl” refers to a structure consisting of two cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” refers to a structure consisting of two cycloalkyl moieties that have exactly one atom in common. Examples include, but are not limited to, spiro[4,5]decyl, spiro[2,3]hexyl, and the like.

The term “heterobicycloalkyl” refers to a bicycloalkyl structure in which at least one carbon atom is replaced with a heteroatom independently selected from oxygen, nitrogen, and sulfur.

The term “heterospiroalkyl” refers to a spiroalkyl structure in which at least one carbon atom is replaced with a heteroatom independently selected from oxygen, nitrogen, and sulfur.

The term “alkylcarbonyloxyalkyl” refers to an ester moiety, for example acetoxymethyl, n-butyryloxyethyl, and the like.

The term “alkynylcarbonyl” refers to an alkynylketo functionality, for example propynoyl and the like.

The term “hydroxyalkyl” refers to an alkyl group substituted with one or more hydroxy groups, for example hydroxymethyl, 2,3-dihydroxybutyl, and the like.

The term “alkylsulfonylalkyl” refers to an alkyl group substituted with an alkylsulfonyl moiety, for example mesylmethyl, isopropylsulfonylethyl, and the like.

The term “alkylsulfonyl” refers to a sulfonyl moiety substituted with an alkyl group, for example mesyl, n-propylsulfonyl, and the like.

The term “acetylaminoalkyl” refers to an alkyl group substituted with an amide moiety, for example acetylaminomethyl and the like.

The term “acetylaminoalkenyl” refers to an alkenyl group substituted with an amide moiety, for example 2-(acetylamino)vinyl and the like.

The term “alkenyl” refers to an ethylenically unsaturated hydrocarbon group, straight or branched chain, having 1 or 2 ethylenic bonds, for example vinyl, allyl, 1-butenyl, 2-butenyl, isopropenyl, 2-pentenyl, and the like.

The term “haloalkenyl” refers to an alkenyl group substituted with one or more halo groups.

Unless otherwise specified, the term “cycloalkenyl” refers to a cyclic aliphatic 3 to 8 ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl, and the like.

The term “alkynyl” refers to an unsaturated hydrocarbon group, straight or branched, having at least one acetylenic bond, for example ethynyl, propargyl, and the like.

The term, “haloalkynyl” refers to an alkynyl group substituted with one or more independent halo groups.

The term “alkylcarbonyl” refers to an alkylketo functionality, for example acetyl. n-butyryl, and the like.

The term “alkenylcarbonyl” refers to an alkenylketo functionality, for example, propenoyl and the like.

The term “aryl” refers to phenyl or naphthyl, which may be optionally substituted. Examples of aryl include, but are not limited to, phenyl, 4-chlorophenyl, 4-fluorophenyl, 4-bromophenyl, 3-nitrophenyl, 2-methoxyphenyl, 2-methylphenyl, 3-methylphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, 3-benzyloxyphenyl, 4-benzyloxyphenyl, 3-benzyloxy-2-fluorophenyl, 7-phenyl-naphthalen-2-yl, 1-fluoro-7-phenyl-naphthalen-2-yl, 8-fluoro-7-phenyl-naphthalen-2-yl, 7-(2-fluorophenyl)naphthalen-2-yl, 7-(pyridin-2-yl)- naphthalen-2-yl, 1-fluoro-7-(pyridin-2-yl)naphthalen-2-yl, and 2-iodo-4-methylphenyl. The aryl ring may be optionally substituted with one or more substituents.

The terms “heteroaryl” or “hetaryl” or “heteroar-” or “hetar-” refer to a substituted or unsubstituted 5- or 6-membered unsaturated ring containing one, two, three, or four independently selected heteroatoms, preferably one or two heteroatoms independently selected from oxygen, nitrogen, and sulfur or to a bicyclic unsaturated ring system containing up to 10 atoms including at least one heteroatom selected from oxygen, nitrogen, and sulfur. Examples of hetaryls include, but are not limited to, 2-, 3- or 4-pyridinyl, pyrazinyl, 2-, 4-, or 5-pyrimidinyl, pyridazinyl, triazolyl, tetrazolyl, imidazolyl, 2- or 3-thienyl, 2- or 3-furyl, pyrrolyl, oxazolyl, isoxazolyl, thiazolyl, isothiazolyl, oxadiazolyl, thiadiazolyl, quinolyl, isoquinolyl, benzimidazolyl, benzotriazolyl, benzofuranyl, benzothienyl, 2-, 3-, 4-, 5-, 6-, or 7-(1H-indolyl), 2-phenyl-quinolin-7-yl, 8-fluoro-2-phenyl-quinolin-7-yl, 8-fluoro-4-methyl-2-phenyl-quinolin-7-yl, and 4-methyl-2-phenyl-quinolin-7-yl. The heterocyclic ring may be optionally substituted with one or more substituents.

The terms “aryl-alkyl” or “arylalkyl” or “aralkyl” are used to describe a group wherein the alkyl chain can be branched or straight chain forming a bridging portion with the terminal aryl, as defined above, of the aryl-alkyl moiety. Examples of aryl-alkyl groups include, but are not limited to, optionally substituted benzyl, phenethyl, phenpropyl and phenbutyl such as 2, 3, or 4-fluoro-benzyl, or 2,3-, 4, 5, or 6-difluoro or trifluorobenzyl, 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxyphenyl)butyl, and 10-phenyldecyl.

The terms “aryl-cycloalkyl” or “arylcycloalkyl” are used to describe a group wherein the terminal aryl group is attached to a cycloalkyl group, for example phenylcyclopentyl and the like.

The terms “aryl-alkenyl” or “arylalkenyl” or “aralkenyl” are used to describe a group wherein the alkenyl chain can be branched or straight chain forming a bridging portion of the aralkenyl moiety with the terminal aryl portion, as defined above, for example styryl (2-phenylvinyl), phenpropenyl, and the like.

The terms “aryl-alkynyl” or “arylalkynyl” or “aralkynyl” are used to describe a group wherein the alkynyl chain can be branched or straight chain forming a bridging portion of the aryl-alkynyl moiety with the terminal aryl portion, as defined above, for example 3-phenyl-1-propynyl, and the like.

The terms “aryl-oxy” or “aryloxy” or “aroxy” are used to describe a terminal aryl group attached to a bridging oxygen atom. Typical aryl-oxy groups include phenoxy, 3,4-dichlorophenoxy, and the like.

The terms “aryl-oxyalkyl” or “aryloxyalkyl” or “aroxyalkyl” are used to describe a group wherein an alkyl group is substituted with a terminal aryl-oxy group, for example pentafluorophenoxymethyl and the like.

The term “heterocycloalkenyl” refers to a cycloalkenyl structure in which at least one carbon atom is replaced with a heteroatom selected from oxygen, nitrogen, and sulfur.

The terms “hetaryl-oxy” or “heteroaryl-oxy” or “hetaryloxy” or “heteroaryloxy” or “hetaroxy” or “heteroaroxy” are used to describe a terminal hetaryl group attached to a bridging oxygen atom. Typical hetaryl-oxy groups include 4,6-dimethoxypyrimidin-2-yloxy and the like.

The terms “hetarylalkyl” or “heteroarylalkyl” or “hetaryl-alkyl” or “heteroaryl-alkyl” or “hetaralkyl” or “heteroaralkyl” are used to describe a group wherein the alkyl chain can be branched or straight chain forming a bridging portion of the heteroaralkyl moiety with the terminal heteroaryl portion, as defined above, for example 3-furylmethyl, thenyl, furfuryl, and the like.

The terms “hetarylalkenyl” or “heteroarylalkenyl” or “hetaryl-alkenyl” or “heteroaryl-alkenyl” or “hetaralkenyl” or heteroaralkenyl” are used to describe a group wherein the alkenyl chain can be branched or straight chain forming a bridging portion of the heteroaralkenyl moiety with the terminal heteroaryl portion, as defined above, for example 3-(4-pyridyl)-1-propenyl.

The terms “hetarylalkynyl” or “heteroarylalkynyl” or “hetaryl-alkynyl” or “heteroaryl-alkynyl” or “hetaralkynyl” or “heteroaralkynyl” are used to describe a group wherein the alkynyl chain can be branched or straight chain forming a bridging portion of the heteroaralkynyl moiety with the heteroaryl portion, as defined above, for example 4-(2-thienyl)-1-butynyl.

The term “heterocyclyl” or “hetcyclyl” refers to a substituted or unsubstituted 4-, 5-, or 6-membered saturated or partially unsaturated ring containing one, two, or three heteroatoms, preferably one or two heteroatoms independently selected from oxygen, nitrogen and sulfur; or to a bicyclic ring system containing up to 10 atoms including at least one heteroatom independently selected from oxygen, nitrogen, and sulfur wherein the ring containing the heteroatom is saturated. Examples of heterocyclyls include, but are not limited to, tetrahydrofuranyl, tetrahydrofuryl, pyrrolidinyl, piperidinyl, 4-pyranyl, tetrahydropyranyl, thiolanyl, morpholinyl, piperazinyl, dioxolanyl, dioxanyl, indolinyl, tetrahydropyridinyl, piperidinyl, and 5-methyl-6-chromanyl.

The terms “heterocyclylalkyl” or “heterocyclyl-alkyl” or “hetcyclylalkyl” or “hetcyclylalkyl” are used to describe a group wherein the alkyl chain can be branched or straight chain forming a bridging portion of the heterocyclylalkyl moiety with the terminal heterocyclyl portion, as defined above, for example 3-piperidinylmethyl and the like.

The terms “heterocyclylalkenyl” or “heterocyclyl-alkenyl” or “hetcyclylalkenyl” or “hetcyclyl-alkenyl” are used to describe a group wherein the alkenyl chain can be branched or straight chain forming a bridging portion of the heterocyclylalkenyl moiety with the terminal heterocyclyl portion, as defined above, for example 2-morpholinyl-1-propenyl and the like.

The terms “heterocyclylalkynyl” or “heterocyclyl-alkynyl” or “hetcyclylalkynyl” or “hetcyclyl-alkynyl” are used to describe a group wherein the alkynyl chain can be branched or straight chain forming a bridging portion of the heterocyclylalkynyl moiety with the terminal heterocyclyl portion, as defined above, for example 2-pyrrolidinyl-1-butynyl and the like.

The term “carboxylalkyl” refers to a terminal carboxyl (—COOH) group attached to branched or straight chain alkyl groups as defined above.

The term “carboxylalkenyl” refers to a terminal carboxyl (—COOH) group attached to branched or straight chain alkenyl groups as defined above.

The term “carboxylalkynyl” refers to a terminal carboxyl (—COOH) group attached to branched or straight chain alkynyl groups as defined above.

The term “carboxylcycloalkyl” refers to a terminal carboxyl (—COOH) group attached to a cyclic aliphatic ring structure as defined above.

The term “carboxylcycloalkenyl” refers to a terminal carboxyl (—COOH) group attached to a cyclic aliphatic ring structure having ethylenic bonds as defined above.

The terms “cycloalkylalkyl” or “cycloalkyl-alkyl” refer to a terminal cycloalkyl group as defined above attached to an alkyl group, for example cyclopropylmethyl, cyclohexylethyl, and the like.

The terms “cycloalkylalkenyl” or “cycloalkyl-alkenyl” refer to a terminal cycloalkyl group as defined above attached to an alkenyl group, for example cyclohexylvinyl, cycloheptylallyl, and the like.

The terms “cycloalkylalkynyl” or “cycloalkyl-alkynyl” refer to a terminal cycloalkyl group as defined above attached to an alkynyl group, for example cyclopropylpropargyl, 4-cyclopentyl-2-butynyl, and the like.

The terms “cycloalkenylalkyl” or “cycloalkenyl-alkyl” refer to a terminal cycloalkenyl group as defined above attached to an alkyl group, for example 2-(cyclopenten-1-yl)ethyl and the like.

The terms “cycloalkenylalkenyl” or “cycloalkenyl-alkenyl” refer to terminal a cycloalkenyl group as defined above attached to an alkenyl group, for example 1-(cyclohexen-3-yl)allyl and the like.

The terms “cycloalkenylalkynyl” or “cycloalkenyl-allynyl” refer to terminal a cycloalkenyl group as defined above attached to an alkynyl group, for example 1-(cyclohexen-3-yl)propargyl and the like.

The term “carboxylcycloalkylalkyl” refers to a terminal carboxyl (—COOH) group attached to the cycloalkyl ring portion of a cycloalkylalkyl group as defined above.

The term “carboxylcycloalkylalkenyl” refers to a terminal carboxyl (—COOH) group attached to the cycloalkyl ring portion of a cycloalkylalkenyl group as defined above.

The term “carboxylcycloalkylalkynyl” refers to a terminal carboxyl (—COOH) group attached to the cycloalkyl ring portion of a cycloalkylalkynyl group as defined above.

The term “carboxylcycloalkenylalkyl” refers to a terminal carboxyl (—COOH) group attached to the cycloalkenyl ring portion of a cycloalkenylalkyl group as defined above.

The term “carboxylcycloalkenylalkenyl” refers to a terminal carboxyl (—COOH) group attached to the cycloalkenyl ring portion of a cycloalkenylalkenyl group as defined above.

The term “carboxylcycloalkenylalkynyl” refers to a terminal carboxyl (—COOH) group attached to the cycloalkenyl ring portion of a cycloalkenylalkynyl group as defined above.

The term “alkoxy” includes both branched and straight chain terminal alkyl groups attached to a bridging oxygen atom. Typical alkoxy groups include methoxy, ethoxy, n-propoxy, isopropoxy, tert-butoxy and the like.

The term “haloalkoxy” refers to an alkoxy group substituted with one or more halo groups, for example chloromethoxy, trifluoromethoxy, difluoromethoxy, perfluoroisobutoxy, and the like.

The term “alkoxyalkoxyalkyl” refers to an alkyl group substituted with an alkoxy moiety which is in turn is substituted with a second alkoxy moiety, for example methoxymethoxymethyl, isopropoxymethoxyethyl, and the like.

The term “alkylthio” includes both branched and straight chain alkyl groups attached to a bridging sulfur atom, for example methylthio and the like.

The term “haloalkylthio” refers to an alkylthio group substituted with one or more halo groups, for example trifluoromethylthio and the like.

The term “alkoxyalkyl” refers to an alkyl group substituted with an alkoxy group, for example isopropoxymethyl and the like.

The term “alkoxyalkenyl” refers to an alkenyl group substituted with an alkoxy group, for example 3-methoxyallyl and the like.

The term “alkoxyalkynyl” refers to an alkynyl group substituted with an alkoxy group, for example 3-methoxypropargyl.

The term “alkoxycarbonylalkyl” refers to a straight chain or branched alkyl substituted with an alkoxycarbonyl, for example ethoxycarbonylmethyl, 2-(methoxycarbonyl)propyl and the like.

The term “alkoxycarbonylalkenyl” refers to a straight chain or branched alkenyl as defined above substituted with an alkoxycarbonyl, for example 4-(ethoxycarbonyl)-2-butenyl and the like.

The term “alkoxycarbonylalkynyl” refers to a straight chain or branched alkynyl as defined above substituted with an alkoxycarbonyl, for example 4-(ethoxycarbonyl)-2-butynyl and the like.

The term “haloalkoxyalkyl” refers to a straight chain or branched alkyl as defined above substituted with a haloalkoxy, for example 2-chloroethoxymethyl, trifluoromethoxymethyl and the like.

The term “haloalkoxyalkenyl” refers to a straight chain or branched alkenyl as defined above substituted with a haloalkoxy, for example 4-(chloromethoxy)-2-butenyl and the like.

The term “haloalkoxyalkynyl” refers to a straight chain or branched alkynyl as defined above substituted with a haloalkoxy, for example 4-(2-fluoroethoxy)-2-butynyl and the like.

The term “alkylthioalkyl” refers to a straight chain or branched alkyl as defined above substituted with an alkylthio group, for example methylthiomethyl, 3-(isobutylthio)heptyl, and the like.

The term “alkylthioalkenyl” refers to a straight chain or branched alkenyl as defined above substituted with an alkylthio group, for example 4-(methylthio)-2-butenyl and the like.

The term “alkylthioalkynyl” refers to a straight chain or branched alkynyl as defined above substituted with an alkylthio group, for example 4-(ethylthio)-2-butynyl and the like.

The term “haloalkylthioalkyl” refers to a straight chain or branched alkyl as defined above substituted with an haloalkylthio group, for example 2-chloroethylthiomethyl, trifluoromethylthiomethyl and the like.

The term “haloalkylthioalkenyl” refers to a straight chain or branched alkenyl as defined above substituted with an haloalkylthio group, for example 4-(chloromethylthio)-2-butenyl and the like.

The term “haloalkylthioalkynyl” refers to a straight chain or branched alkynyl as defined above substituted with a haloalkylthio group, for example 4-(2-fluoroethylthio)-2-butynyl and the like.

The term “dialkoxyphosphorylalkyl” refers to two straight chain or branched alkoxy groups as defined above attached to a pentavalent phosphorous atom, containing an oxo substituent, which is in turn attached to an alkyl, for example diethoxyphosphorylmethyl and the like.

One in the art understands that an “oxo” requires a second bond from the atom to which the oxo is attached. Accordingly, it is understood that oxo cannot be subststituted onto an aryl or heteroaryl ring.

The term “oligomer” refers to a low-molecular weight polymer, whose number average molecular weight is typically less than about 5000g/mol, and whose degree of polymerization (average number of monomer units per chain) is greater than one and typically equal to or less than about 50.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The invention also encompasses a pharmaceutical composition that is comprised of a compound of Formula I in combination with a pharmaceutically acceptable carrier.

Preferably the composition is comprised of a pharmaceutically acceptable carrier and a non-toxic therapeutically effective amount of a compound of Formula I as described above (or a pharmaceutically acceptable salt thereof).

Moreover, within this preferred embodiment, the invention encompasses a pharmaceutical composition for the treatment of disease by inhibiting kinases, comprising a pharmaceutically acceptable carrier and a non-toxic therapeutically effective amount of compound of Formula I as described above (or a pharmaceutically acceptable salt thereof).

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids. Particularly preferred are formic and hydrochloric acid.

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or a pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration. e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5g of the active ingredient and each cachet or capsule preferably containing from about 0.05 mg to about 5g of the active ingredient.

For example, a formulation intended for the oral administration to humans may contain from about 0.5 mg to about 5g of active agent, compounded with an appropriate and convenient amount of carrier material which may vary from about 5 to about 95 percent of the total composition. Unit dosage forms will generally contain between from about 1 mg to about 2g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7g per patient per day. For example, inflammation, cancer, psoriasis, allergy, asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

Biological Assays

1. ROCK Kinase Assay

cDNA encoding a chimeric ROCK kinase protein was cloned into baculovirus expression vectors for protein expression as N-terminal or C-terminal fusion proteins with His₆ in insect cells. The expressed protein comprises residues 2-238 of ROCK1 fused to residues 255-548 of ROCK2. Following purification to greater than 90% homogeneity using a Nickel affinity resin, the enzyme was used in fluorescence polarization-based kinase assays (IMAP) to determine the ability of compounds to inhibit phosphorylation of a fluorescent-tagged substrate peptide based on a sequence within ribosomal protein S6 (Molecular Devices #R7229).

Kinase activity is determined in a 384-well homogeneous IMAP fluorescence polarization-based assay that measures the ability of ROCK to phosphorylate a fluorescent-tagged peptide substrate based on a sequence within ribosomal protein S6 (Molecular Devices #R7229) in the presence of ATP. Substrate phosphorylation is monitored following addition of IMAP nanoparticles (comprising trivalent metal cations that bind specifically to phosphate groups), which bind to the phosphorylated peptide molecules and decrease their molecular mobility. This effect is quantitated using a fluorescence polarization detector to monitor the highly polarized fluorescence emission from the bound phosphorylated molecules following excitation with polarized light.

The stock reagents used in the assay are as follows:

Kinase Reaction Buffer: 10 mM Tris HCl (pH 7.2), 10 mM MgCl₂, 0.1% BSA, 0.05% NaN₃, 1 mM DTT (added fresh).

Fluorescent peptide: Molecular Devices #R7229 (FAM-S6 derived peptide).

IMAP Progressive Binding Buffer System: (Molecular Devices #R8125)

Assay Protocol

Compounds are diluted in DMSO and Kinase Reaction Buffer to generate serial dilutions containing compound stocks at 4× final concentration containing 4% DMSO. 5 μl of diluted compound (or 4% DMSO for control wells) are added to each well in a 384-well assay plate (e.g. Costar #3710). The substrate peptide is diluted to 200 nM in Kinase Reaction Buffer, either in the presence or absence of ATP at 2× final concentration (e.g. 2-200 μM ATP), and 10 μL is added per well. 5 μL ROCK enzyme (16 ng/well), diluted in Kinase Reaction Buffer, is then added to all wells to initiate the reaction. The phosphorylation reaction is conducted at room temperature, and terminated by the addition of 23 μl of the Progressive Binding Buffer (Molecular Devices, #R8125), containing a 1:1000 dilution of IMAP nanoparticles (Molecular Devices). Following incubation for 1 hour at room temperature, the degree of substrate phosphorylation is quantitated using an Analyst plate reader in fluorescence polarization mode.

Comparison of the fluorescence polarization obtained in the presence of compound with those of controls (in the presence and absence of ATP, with no compound added), allows the degree of inhibition of kinase activity to be determined over a range of compound concentrations. These inhibition values are fitted to a sigmoidal dose-response inhibition curve to determine the IC₅₀ values (i.e. the concentration of compound that reduces the kinase activity to 50% of the control activity).

The compounds of this invention reduced the ability of ROCK to phosphorylate the substrate peptide (Molecular Devices #R7229) in the above assay, thus demonstrating direct inhibition of the ROCK Ser/Thr kinase activity. IC₅₀ values in this assay were between 5 nM and 10 μM.

Compounds of this invention also inhibited the tyrosine kinase activity of FAK. IC₅₀ values were between 0.5 μM and 30 μM.

Compounds of this invention also inhibited the tyrosine kinase activity of CSF-1R, Ret, KDR, Kit, IGF-1R, RON, Met, EGFR, Alk, Flt3 with IC₅₀ values less than 10 μM.

Compounds of this invention also inhibited the serine/threonine kinase activity of PDK1, Akt, CDK2, IKKb, MEK1, PKN1, PKA, PKC, RSK1, p70S6K, SGK, Aurora-A with IC₅₀ values less than 10 μM.

### EXPERIMENTAL

Schemes 1-5 below, as well as the experimental procedures that follow, show how to synthesize compounds of this invention and utilize the following abbreviations: Me for methyl, Et for ethyl, ⁱPr or ⁱPr for isopropyl, n-Bu for n-butyl, t-Bu for tert-butyl, Ac for acetyl, Ph for phenyl, 4Cl—Ph or (4Cl)Ph for 4-chlorophenyl, 4Me-Ph or (4Me)Ph for 4-methylphenyl, (p-CH₃O)Ph for p-methoxyphenyl, (p-NO₂)Ph for p-nitrophenyl, 4Br—Ph or (4Br)Ph for 4-bromophenyl, 2-CF₃—Ph or (2CF₃)Ph for 2-trifluoromethylphenyl, DMAP for 4-(dimethylamino)pyridine, DCC for 1,3-dicyclohexylcarbodiimide, EDC for 1-(3-dimethylaminopropyl)-3-ethylcarbodiimide hydrochloride, HOBt for 1-hydroxybenzotriazole, HOAt for 1-hydroxy-7-azabenzotriazole, CDI for 1,1′-carbonyldiimidazole, NMO for 4-methylmorpholine N-oxide, DEAD for diethyl azodicarboxylate, DIAD for diisopropyl azodicarboxylate, DBAD for di-tert-butyl azodicarboxylate, HPFC for high performance flash chromatography, rt for room temperature, min for minute, h for hour, Bn for benzyl, DMF for N,N-dimethylformamide, DMA for N,N-dimethylacetamide, NMP for N-methylpyrrolidinone, DCE for 1,2-dichloroethane, K₂CO₃ for potassium carbonate, Cs₂CO₃ for cesium carbonate, Ag₂CO₃ for silver carbonate, NaH for sodium hydride.

Accordingly, the following are compounds which are useful as intermediates in the formation of kinase inhibiting Examples. The compounds of Formula I of this invention and the intermediates used in the synthesis of the compounds of this invention were prepared according to the following methods.

Method A was used when preparing compounds of Formula I-A (Compounds of Formula I wherein X¹, X², X³, and X⁴ equals CH and X⁵═NH) as shown below in Scheme 1:

Method A: 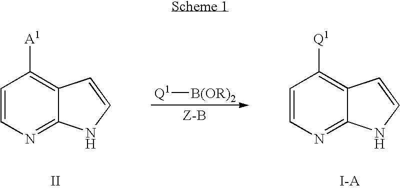
where Q¹ is a suitably substituted aryl, heteroaryl, or heterocyclyl group represented by (Z¹)ₙ-(Y¹)ₘ—R¹ described previously; A¹=halogen such as Cl, Br, or I; B(OR)₂=suitable boronic acid/ester.

In a typical preparation of compounds of Formula I-A, compound of Formula II was reacted with a suitable boronic acid/ester (Q¹-B(OR)₂) in a suitable solvent via typical Suzuki coupling procedures. Suitable solvents for use in the above process included, but were not limited to, ethers such as tetrahydrofuran (THF), glyme, dioxane, dimethoxyethane, and the like; dimethylformamide (DMF); dimethyl sulfoxide (DMSO); acetonitrile; alcohols such as methanol, ethanol, isopropanol, trifluoroethanol, and the like; and chlorinated solvents such as methylene chloride (CH₂Cl₂) or chloroform (CHCl₃). If desired, mixtures of these solvents were used, however, the preferred solvent was dimethoxyethane/water. The above process was carried out at temperatures between about −78° C. and about 120° C. Preferably, the reaction was carried out between 60° C. and about 10° C. The above process to produce compounds of the present invention was preferably carried out at about atmospheric pressure although higher or lower pressures were used if desired. Substantially equimolar amounts of reactants were preferably used although higher or lower amounts were used if desired.

One skilled in the art will appreciate that alternative methods may be applicable for preparing compounds of Formula I-A from II. For example, compound of Formula II could be reacted with a suitable organotin reagent Q¹-SnBu₃ or the like in a suitable solvent via typical Stille coupling procedures. Additionally, one skilled in the art would appreciate that A¹ can equal B(OR)₂ and coupled via the Suzuki reaction to Q¹-halo, where halo=Cl, Br, I, or OTf, to afford compound of Formula I-A, via conditions described herein.

Method B was used when converting compound of Formula I-B (compounds of Formula I-A wherein Q¹=Z¹-CO—R^{6a}) to compounds of Formula I-C (compounds of Formula I-A wherein Q¹=Z¹-CR^{6a}R⁷a(OH)) and I-D (compounds of Formula I-A wherein Q¹=Z¹-CH(R^{6a})(NR¹R⁶)) shown below in Scheme 2:

Method B: 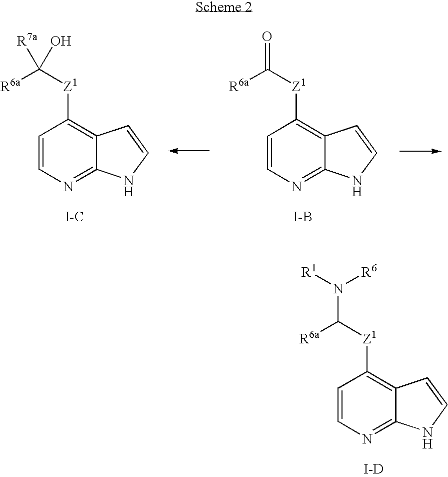
where Z¹, R¹, R⁶, R^{6a}, and R^{7a} are as defined previously for compound of Formula I.

In a typical preparation of compound of Formula I-C when R^{7a}═H, compound of Formula I-B was reduced with a suitable reducing agent in a suitable solvent, such as but not limited to sodium borohydride in methanol. In a typical preparation of compound of Formula I-C when R^{7a} equals a group other than H, such as but not limited to alkyl, aryl, heteroaryl, aralkyl, heteroaralkyl, cycloalkyl, or heterocycloalkyl, compound of Formula I-B was reacted with a suitable nucleophilic reagent such as R^{7a}MgBr or R^{7a}L¹ in a suitable solvent such as but not limited to THF. Compounds of Formula I-D can be reacted with various NR¹R⁶ groups under typical reductive amination conditions (NaBH₃CN or NaBH(OAc)₃ with HNR¹R⁶ in a suitable solvent, such as but not limited to ethers such as THF, and under suitable reaction conditions. The above processes were carried out at temperatures between about −78° C. and about 120° C. Preferably, the reaction was carried out between 0° C. and about 80° C. The above processes to produce compounds of the present invention were preferably carried out at about atmospheric pressure although higher or lower pressures were used if desired. Substantially equimolar amounts of reactants were preferably used although higher or lower amounts were used if desired.

Method C was used when converting compound of Formula I-E (compounds of Formula I-A wherein Q¹=Z¹-NHR⁶) to compounds of Formula I-F (compounds of Formula I-A wherein Q¹=Z¹-NR⁶(COR¹)) and I-G (compounds of Formula I-A wherein Q¹=Z¹-NR⁶(CONR¹R^{6a})) shown below in Scheme 3:

Method C: 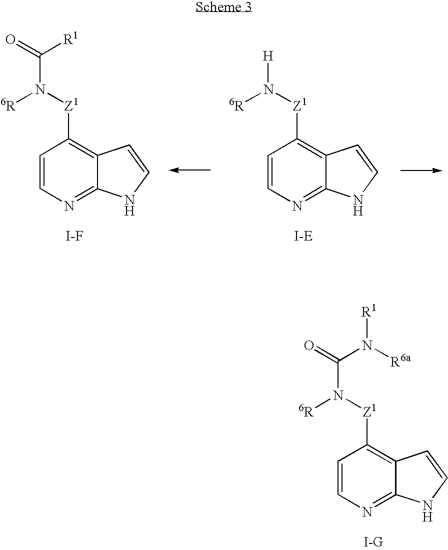
where Z¹, R¹, R⁶, and R^{6a} are as defined previously for compound of Formula I.

In a typical preparation, of a compound of Formula I-F, a compound of Formula I-E is reacted with A²-CO—R¹ under suitable conditions for conversion of an amine to an amide (A²=suitable leaving group such as Cl, N-hydroxysuccinimide or OH). Suitable conditions included but are not limited to treating compounds of Formula I-E and A²-CO—R¹ (when A²═OH) with coupling reagents such as DCC or EDC in conjunction with DMAP, HOBt, HOAt and the like. Suitable solvents for use in the above process included, but were not limited to, ethers such as tetrahydrofuran (THF), glyme, and the like; dimethylformamide (DMF); dimethyl sulfoxide (DMSO); acetonitrile; halogenated solvents such as chloroform or methylene chloride. If desired, mixtures of these solvents were used, however the preferred solvent was methylene chloride. The above process was carried out at temperatures between about 0° C. and about 80° C. Preferably, the reaction was carried out at about 22° C. The above process to produce compounds of the present invention was preferably carried out at about atmospheric pressure although higher or lower pressures were used if desired. Substantially, equimolar amounts of reactants were preferably used although higher or lower amounts were used if desired. Additionally, other suitable reaction conditions for the conversion of an amine to an amide can be found in Larock, R. C. Comprehensive Organic Transformations, 2^{nd} ed.; Wiley and Sons: New York, 1999, pp 1941-1949.

In a typical preparation, of a compound of Formula I-G, a compound of Formula I-E is reacted with A³-CO—NR¹R^{6a} or a suitable isocyanate, CO(NR¹R^{6a}), under suitable conditions for conversion of an amine to a urea (A³=suitable leaving group such as Cl or p-nitro-phenoxide). Suitable solvents for use in the above process included, but were not limited to, ethers such as tetrahydrofuran (THF), glyme, and the like; dimethylformamide (DMF); dimethyl sulfoxide (DMSO); acetonitrile; halogenated solvents such as chloroform or methylene chloride. If desired, mixtures of these solvents were used, however the preferred solvent was THF. The above process was carried out at temperatures between about 0° C. and about 80° C. Preferably, the reaction was carried out at about 22° C. The above process to produce compounds of the present invention was preferably carried out at about atmospheric pressure although higher or lower pressures were used if desired. Substantially, equimolar amounts of reactants were preferably used.

Method D was used when converting compound of Formula I-H (compounds of Formula I-A wherein Q¹=Z¹-CO₂-L¹) to compounds of Formula I-J (compounds of Formula I-A wherein Q¹=Z¹-CO—NR¹R⁶) as shown below in Scheme 4:

Method D: 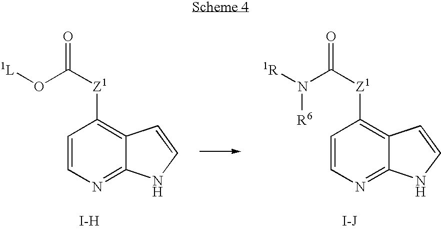
where Z¹, R¹, and R⁶ are as defined previously for compound of Formula I and L¹ is lower alkyl, aralkyl or H.

In a typical preparation of compound of Formula I-J, compound of Formula I-H and HNR¹R⁶ were reacted under suitable amide coupling conditions. Suitable conditions included but are not limited to treating compounds of Formula I-H (when L¹=H) with HNR¹R⁶ and coupling reagents such as DCC or EDC in conjunction with DMAP, HOBt, HOAt and the like. Suitable solvents for use in the above process included, but were not limited to, ethers such as tetrahydrofuran (THF), glyme, and the like; dimethylformamide (DMF); dimethyl sulfoxide (DMSO); acetonitrile; halogenated solvents such as chloroform or methylene chloride. If desired, mixtures of these solvents were used, however the preferred solvent was methylene chloride. The above process was carried out at temperatures between about 0° C. and about 80° C. Preferably, the reaction was carried out at about 22° C. The above process to produce compounds of the present invention was preferably carried out at about atmospheric pressure although higher or lower pressures were used if desired. Substantially, equimolar amounts of reactants were preferably used although higher or lower amounts were used if desired. When L¹=alkyl, conversion to L¹=H can occur through treatment under typical saponification conditions such as but not limited to KOH, NaOH, NaHCO₃, Na₂CO₃, in the presence of water and a co-solvent such as methanol or THF.

Alternatively, compounds of Formula I-J could be prepared by first converting compounds of Formula I-H (when OL¹=OH) to an acid chloride (where OL¹=Cl) by treatment with SOCl₂, oxalyl chloride, or similar reagents known to convert a carboxylic acid to an acid chloride, followed by reaction with HNR¹R⁶ along with a suitable base such as triethylamine or ethyldiisopropylamine and the like in conjunction with DMAP and the like. Suitable solvents for use in this process included, but were not limited to, ethers such as tetrahydrofuran (THF), glyme, and the like; dimethylformamide (DMF); dimethyl sulfoxide (DMSO); acetonitrile; halogenated solvents such as chloroform or methylene chloride. If desired, mixtures of these solvents were used, however the preferred solvent was methylene chloride. The above process was carried out at temperatures between about −20° C. and about 40° C. Preferably, the reaction was carried out between 0° C. and 25° C. The above process to produce compounds of the present invention was preferably carried out at about atmospheric pressure although higher or lower pressures were used if desired. Additionally, when L¹=alkyl such as methyl or ethyl, treatment of the ester with a prepared solution of AlMe₃ and HNR¹R⁶ (typical Weinreb amidation conditions) afforded conversion of CO₂L¹ to CO(NR¹R⁶). Additionally, other suitable reaction conditions for the conversion of an acid to an amide can be found in Larock, R. C. Comprehensive Organic Transformations, 2^{nd} ed.; Wiley and Sons: New York, 1999, pp 1941-1949.

Method E was used when converting compounds of Formula I-K (compounds of Formula I-A wherein Q¹=Z¹-C(R^{6a}R^{7a})N(R⁶)-L²) to compound of Formula I-L (compounds of Formula I-A wherein Q¹=Z¹-C(R^{6a}R^{7a})N(R⁶)—H) and then compounds of Formula I-L to compounds of Formula I-M (compounds of Formula I-A wherein Q¹=Z¹-C(R^{6a}R^{7a})N(R⁶)CO—R¹) as shown below in Scheme 5:

Method E: 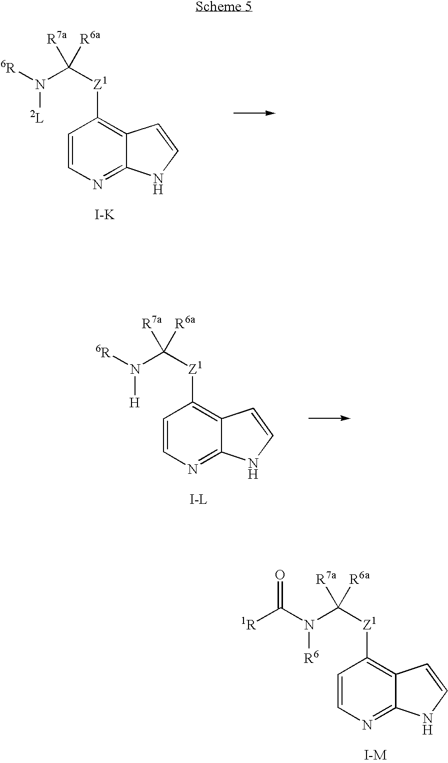
where Z¹, R¹, R⁶, R^{6a}, and R^{7a} are as defined previously for compound of Formula I and L² is a suitable protecting group such as Boc.

In a typical preparation of compound of Formula I-L, compound of Formula I-K is reacted under acidic conditions in a suitable solvent. Acidic conditions include but are not limited to treating compounds of Formula I-K (when L²=Boc) with TFA or HCl. Suitable solvents for use in the above process included, but were not limited to, ethers such as tetrahydrofuran (THF), glyme, and the like; dimethylformamide (DMF); dimethyl sulfoxide (DMSO); acetonitrile; halogenated solvents such as chloroform or methylene chloride. If desired, mixtures of these solvents were used. The preferred conditions involved treating compound of Formula I-K with 8M HCl in dioxane in methylene chloride. The above process was carried out at temperatures between about 0° C. and about 80° C. Preferably, the reaction was carried out at about 22° C. The above process to produce compounds of the present invention was preferably carried out at about atmospheric pressure although higher or lower pressures were used if desired. Substantially, equimolar amounts of reactants were preferably used although higher or lower amounts were used if desired. Compound of Formula I-M can be prepared from compounds of Formula I-L following typical amide coupling procedures described previously in Scheme 3 for the conversion of compounds of Formula I-E to I-F.

It would be appreciated by those skilled in the art that in some situations, a substituent that is identical or has the same reactivity to a functional group which has been modified in one of the above processes, will have to undergo protection followed by deprotection to afford the desired product and avoid undesired side reactions. Alternatively, another of the processes described within this invention may be employed in order to avoid competing functional groups. Examples of suitable protecting groups and methods for their addition and removal may be found in the following reference: “Protective Groups in Organic Syntheses”, T. W. Green and P. G. M. Wutz, John Wiley and Sons, 1989.

The following examples are intended to illustrate and not to limit the scope of the present invention.

General Experimental Information:

All melting points were determined with a Me1-Temp II apparatus and are uncorrected. Commercially available anhydrous solvents and HPLC-grade solvents were used without further purification. ¹H NMR and ¹³C NMR spectra were recorded with Varian or Bruker instruments (400 MHz for ¹H, 100.6 MHz for ¹³C) at ambient temperature with TMS or the residual solvent peak as internal standards. The line positions or multiplets are given in ppm (δ) and the coupling constants (J) are given as absolute values in Hertz, while the multiplicities in ¹H NMR spectra are abbreviated as follows: s (singlet), d (doublet), t (triplet), q (quartet), quint (quintet), m (multiplet), m_{c} (centered multiplet), br (broadened), AA′BB′. The signal multiplicities in ¹³C NMR spectra were determined using the DEPT135 pulse sequence and are abbreviated as follows: +(CH or CH₃), −(CH₂), C_{quart} (C). LC/MS analysis was performed using a Gilson 215 autosampler and Gilson 819 autoinjector attached to a Hewlett Packard HP1100 and a MicromassZQ mass spectrometer (also referred to as “OpenLynx”), or a Hewlett Packard HP1050 and a Micromass Platform II mass spectrometer. Both setups used XTERRA MS C18 5μ 4.6×50 mm columns with detection at 254 nm and electrospray ionization in positive mode. For mass-directed purification (MDP), a Waters/Micromass system was used.

Analytical HPLC Conditions:

Unless otherwise stated, all HPLC analyses were run on a Micromass system with a XTERRA MS C18 5μ 4.6×50 mm column and detection at 254 nm. Table A below lists the mobile phase, flow rate, and pressure.

TABLE A

| Time | | 0.01% HCOOH | | |
|---|---|---|---|---|
| (min) | % CH₃CN | in H₂O % | Flow (mL/min) | Pressure (psi) |
| 0.00 | 5 | 95 | 1.3 | 400 |
| 4.00 | 100 | 0 | 1.3 | 400 |
| 5.50 | 100 | 0 | 1.3 | 400 |
| 6.00 | 5 | 95 | 1.3 | 400 |
| 7.00 | 5 | 95 | 1.3 | 400 |

Semipreparative HPLC Conditions:

Where indicated as “purified by Gilson HPLC”, the compounds of interest were purified by a preparative/semipreparative Gilson HPLC workstation with a Phenomenex Luna 5μ C₁₈ (2) 60×21.2 MM 5μ column and Gilson 215 liquid handler (806 manometric module, 811C dynamic mixer, detection at 254 nm). Table B lists the gradient, flow rate, time, and pressure.

TABLE B

| Time | | 0.01% HCOOH | | |
|---|---|---|---|---|
| (min) | % CH₃CN | in H₂O % | Flow (mL/min) | Pressure (psi) |
| 0.00 | 5 | 95 | 15 | 1000 |
| 15.00 | 60 | 40 | 15 | 1000 |
| 15.10 | 100 | 0 | 15 | 1000 |
| 19.00 | 100 | 0 | 15 | 1000 |
| 20.00 | 5 | 95 | 15 | 1000 |

### Example 1

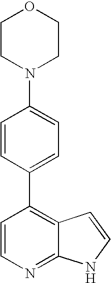

### 4-(4-Morpholin-4-yl-phenyl)-1H-pyrrolo[2,3-b]pyridine

A mixture of 4-chloro-7-azaindole (50 mg, 0.33 mmole) in a mixture of dioxane (4 mL) and water (1 mL) in a 25 mL, two-necked round bottomed flask was charged with K₂CO₃ (27 mg, 0.20 mmole), 4-(morpholino)phenylboronic acid (75 mg, 0.36 mmole), Pd(dppf)₂Cl₂.CH₂Cl₂ catalyst (13 mg, 0.016 mmole). Nitrogen was bubbled into the reaction mixture for 15 min at rt and then heated at 100° C. overnight under nitrogen atmosphere. The reaction mixture was cooled to rt and added triethylamine (3 mL) and evaporated to dryness and purified by column chromatography. The crude was taken in 1% methanol in methylene chloride and loaded onto the column. The column was eluted with 50% ethyl acetate in methylene chloride to remove all the impurities and then polarity increased to 75% EtOAc in methylene chloride. The desired fractions from the column were collected and the resulting solid was triturated with hot isopropyl ether, cooled to rt and filtered to give the title compound as a pale yellow solid. ¹H NMR (DMSO-d₆) δ 3.19 (t, 4H, J=4.5 Hz), 3.75 (t, 4H, J=4.5 Hz), 6.61 (m, 1H), 7.09 (d, 2H, J=8.7 Hz), 7.11 (d, 1H, J=5.1 Hz), 7.48 (t, 1H, J=2.8 Hz), 7.66 (d, 2H, J=9 Hz), 8.21 (d, 1H, J=4.8 Hz), 11.67 (brs, 1H); MS (ES+): m/z 280.14 [MH+].

### Example 2

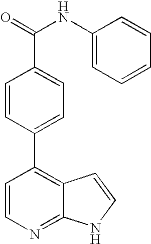

### N-Phenyl-4-(1H-pyrrolo[2,3-b]pyridin-4-yl)-benzamide

Prepared according to the procedure described in EXAMPLE 1 using 4-(phenylcarbamoyl)phenylboronic acid in place of 4-(morpholino)phenylboronic acid. MS (ES+): m/z 314.19 [MH+].

### Example 3

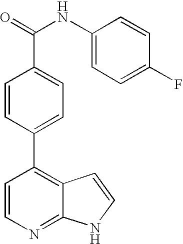

### N-(4-Fluoro-phenyl)-4-(1H-pyrrolo[2,3-b]pyridin-4-yl)-benzamide

Prepared according to the procedure described in EXAMPLE 1 using 4-(4-fluoro-phenylcarbamoyl)phenylboronic acid in place of 4-(morpholino)phenylboronic acid. MS (ES+): m/z 332.13 [MH+].

### Example 4

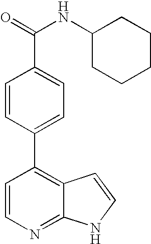

### N-Cyclohexyl-4-(1H-pyrrolo[2,3-b]pyridin-4-yl)-benzamide

Prepared according to the procedure described in Example 1 using 4-(cyclohexylcarbamoyl)phenylboronic acid in place of 4-(morpholino)phenylboronic acid. MS (ES+): m/z 320.24 [MH+].

### Example 5

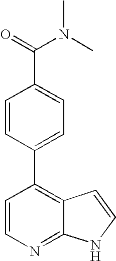

### N,N-Dimethyl-4-(1H-pyrrolo[2,3-b]pyridin-4-yl)-benzamide

Prepared according to the procedure described in EXAMPLE 1 using 4-(dimethylcarbamoyl)phenylboronic acid in place of 4-(morpholino)phenylboronic acid. MS (ES+): m/z 266.18 [MH+].

### Example 6

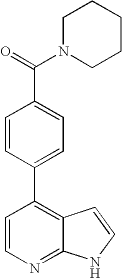

### Piperidin-1-yl-[4-(1H-pyrrolo[2,3-b]pyridin-4-yl)-phenyl]-methanone

Prepared according to the procedure described in EXAMPLE 1 using 4-(piperidine-1-carbonyl)phenylboronic acid in place of 4-(morpholino)phenylboronic acid. MS (ES+): m/z 306.18 [MH+].

### Example 7

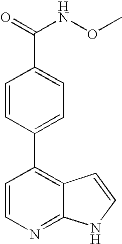

### N-Methoxy-4-(1H-pyrrolo[2,3-b]pyridin-4-yl)-benzamide

Prepared according to the procedure described in EXAMPLE 1 using 4-(methoxycarbamoyl)phenylboronic acid in place of 4-(morpholino)phenylboronic acid. MS (ES+): m/z 268.19 [MH+].

### Example 8

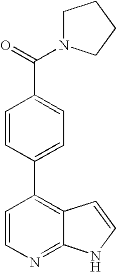

### Pyrrolidin-1-yl-[4-(1H-pyrrolo[2,3-b]pyridin-4-yl)-phenyl]-methanone

Prepared according to the procedure described in EXAMPLE 1 using 4-(pyrrolidine-1-carbonyl)phenylboronic acid in place of 4-(morpholino)phenylboronic acid. MS (ES+): m/z 292.17 [MH+].

### Example 9

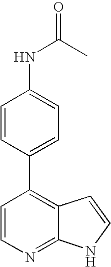

### N-[4-(1H-Pyrrolo[2,3-b]pyridin-4-yl)-phenyl]-acetamide

Prepared according to the procedure described in EXAMPLE 1 using 4-(acetylamino) phenylboronic acid in place of 4-(morpholino)phenylboronic acid. MS (ES+): m/z 252.17 [MH+].

### Example 10

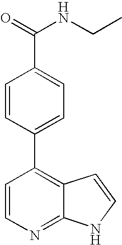

### N-Ethyl-4-(1H-pyrrolo[2,3-b]pyridin-4-yl)-benzamide

Prepared according to the procedure described in EXAMPLE 1 using 4-(ethylcarbamoyl)phenylboronic acid in place of 4-(morpholino)phenylboronic acid. MS (ES+): m/z 266.24 [MH+].

### Example 11

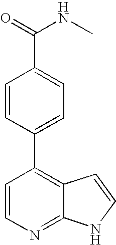

### N-Methyl-4-(1H-pyrrolo[2,3-b]pyridin-4-yl)-benzamide

Prepared according to the procedure described in EXAMPLE 1 using 4-(methylcarbamoyl)phenylboronic acid in place of 4-(morpholino)phenylboronic acid. MS (ES+): m/z 252.22 [MH+].

### Example 12

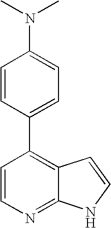

### Dimethyl-[4-(1H-pyrrolo[2,3-b]pyridin-4-yl)-phenyl]-amine

Prepared according to the procedure described in EXAMPLE 1 using 4-(dimethylamino)phenylboronic acid in place of 4-(morpholino)phenylboronic acid. MS (ES+): m/z 238.23 [MH+].

### Example 13

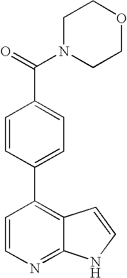

### Morpholin-4-yl-[4-(1H-pyrrolo[2,3-b]pyridin-4-yl)-phenyl]-methanone

Prepared according to the procedure described in EXAMPLE 1 using 4-(morpholine-4-carbonyl)phenylboronic acid in place of 4-(morpholino)phenylboronic acid. MS (ES+): m/z 308.14 [MH+].

### Example 14

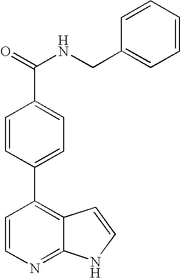

### N-Benzyl-4-(1H-pyrrolo[2,3-b]pyridin-4-yl)-benzamide

Prepared according to the procedure described in EXAMPLE 1 using 4-(benzylcarbamoyl)phenylboronic acid in place of 4-(morpholino)phenylboronic acid. MS (ES+): m/z 328.14 [MH+].

### Example 15

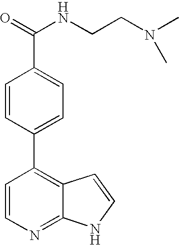

### N-(2-Dimethylamino-ethyl)-4-(1H-pyrrolo[2,3-b]pyridin-4-yl)-benzamide

Prepared according to the procedure described in EXAMPLE 1 using 4-(2-dimethylamino-ethylcarbamoyl)phenylboronic acid in place of 4-(morpholino)phenylboronic acid. MS (ES+): m/z 309.21 [MH+].

### Example 16

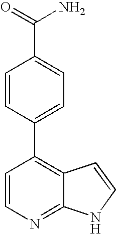

### 4-(1H-Pyrrolo[2,3-b]pyridin-4-yl)-benzamide

Prepared according to the procedure described in EXAMPLE 1 using 4-carbamoylphenylboronic acid in place of -4-(morpholino)phenylboronic acid. MS (ES+): m/z 238.11 [MH+].

### Example 17

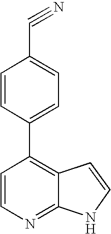

### 4-(1H-Pyrrolo[2,3-b]pyridin-4-yl)-benzonitrile

Prepared according to the procedure described in EXAMPLE 1 using 4-cyanophenylboronic acid in place of 4-(morpholino)phenylboronic acid. MS (ES+): m/z 220.15 [MH+].

### Example 18

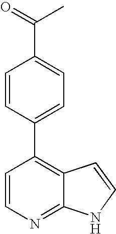

### 1-[4-(1H-Pyrrolo[2,3-b]pyridin-4-yl)-phenyl]-ethanone

Prepared according to the procedure described in EXAMPLE 1 using 4-acetylphenylboronic acid in place of 4-(morpholino)phenylboronic acid. MS (ES+): m/z 237.14 [MH+].

### Example 19

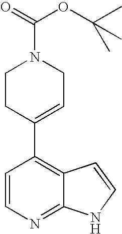

### 4-(1H-Pyrrolo[2,3-b]pyridin-4-yl)-3,6-dihydro-2H-pyridine-1-carboxylic acid tert-butyl ester

Prepared according to the procedure described in EXAMPLE 1 using 4-(4,4,5,5-tetramethyl-[1,3,2]dioxaborolan-2-yl)-3,6-dihydro-2H-pyridine-1-carboxylic acid tert-butyl ester in place of 4-(morpholino)phenylboronic acid. MS (ES+): m/z 300.06 [MH+].

### Example 20

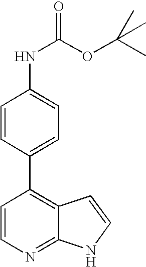

### [4-(1H-Pyrrolo[2,3-b]pyridin-4-yl)-phenyl]-carbamic acid tert-butyl ester

A mixture of 4-chloro-7-azaindole (2.64 g, 17.4 mmole) in dioxane (80 mL) and water (20 mL) in a 250 mL, two-necked round bottomed flask was charged with K₂CO₃ (1.42 g, 10.3 mmole), (4-BOC-aminophenyl)boronic acid (4.74 g, 20 mmole), and Pd(dppf)₂Cl₂.CH₂Cl₂ catalyst (685 mg, 0.84 mmole). Nitrogen was bubbled into the reaction mixture for 15 min at rt and then heated at 100° C. overnight under nitrogen atmosphere. The reaction mixture was cooled to rt and added triethylamine (10 mL) and evaporated to dryness and purified by column chromatography. The crude was taken in methylene chloride and loaded onto the column. The column was eluted with 20-30% ethyl acetate in methylene chloride and the desired fractions from column were collected and the resulting solid was triturated with hot isopropyl ether, cooled to rt and filtered to give the title compound as a pale yellow solid. ¹H NMR (DMSO-d₆): δ 1.49 (s, 9H), 6.61 (m, 1H), 7.13 (d, 1H, J=5.1 Hz), 7.5 (t, 1H, J=2.85 Hz), 7.65 (m, 4H), 8.23 (d, 1H, J=4.8 Hz), 9.53 (s, 1H), 11.71 (brs, 1H); MS (ES+): m/z 310.20 [MH+].

### Example 21

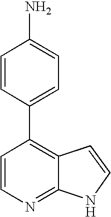

### 4-(1H-Pyrrolo[2,3-b]pyridin-4-yl)-phenylamine

To a cold solution of [4-(1H-pyrrolo[2,3-b]pyridin-4-yl)-phenyl]-carbamic acid tert-butyl ester (3.09 g, 10 mmole) in methylene chloride (40 mL) was added 8N HCl solution of 1,4-dioxane (5 mL, 40 mmole), the resulting mixture was stirred at rt overnight. The resulting solid was collected by filtration and washed with diethyl ether. The solid (2.74 g, 97%) was taken in aqueous sodium carbonate solution and stirred for 10 min and then extracted with ethyl acetate. The ethyl acetate layer was dried over Na₂SO₄, filtered and concentrated. The solid was triturated with hexane and filtered to give the title compound. ¹H NMR (DMSO-d₆): δ 5.38 (s, 2H), 6.65 (m, 1H), 6.69 (d, 2H, J=8.4 Hz), 7.04 (d, 1H, J=4.8 Hz), 7.43 (t, 1H, J=3.0), 7.48 (d, 2H, J=8.7 Hz), 8.15 (d, 1H, J=5.1 Hz), 11.59 (brs, 1H); MS (ES+): m/z 210.12 [MH+].

### Example 22

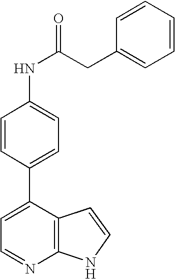

### 2-Phenyl-N-[4-(1H-pyrrolo[2,3-b]pyridin-4-yl)-phenyl]-acetamide

A solution of 4-(1H-pyrrolo[2,3-b]pyridin-4-yl)-phenylamine (50 mg, 0.239 mmole), EDC.HCl (55 mg, 0.286 mmole) and HOBt (32 mg, 0.239 mmole) in methylene chloride (5 mL) was charged with N,N-diisopropylethylamine (62 mg, 0.478 mmole) and phenylactic acid (33 mg, 0.239 mmole). The reaction mixture was stirred at rt overnight. The precipitated solid was collected by filtration and washed with water to afford the title compound. ¹H NMR (DMSO-d₆): δ 3.68 (s, 2H), 6.61 (m, 1H), 7.14 (d, 1H, J=5.4 Hz), 7.25 (m, 1H), 7.33 (m, 4H), 7.50 (t, 1H, J=3.0 Hz), 7.75 (q, 4H), 8,24 (d, 1H, J=4.8 Hz), 10.35 (s, 1H), 11.73 (brs, 1H); MS (ES+): m/z 327.66 [MH+].

### Example 23

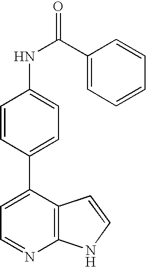

### N-[4-(1H-Pyrrolo[2,3-b]pyridin-4-yl)-phenyl]-benzamide

Prepared according to the procedure described in EXAMPLE 22 using benzoic acid in place of phenylactic acid. MS (ES+): m/z 314.06 [MH+].

### Example 24

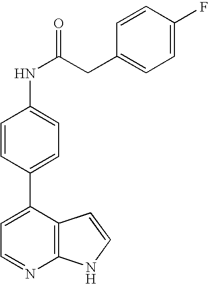

### 2-(4-Fluoro-phenyl)-N-[4-(1H-pyrrolo[2,3-b]pyridin-4-yl)-phenyl]-acetamide

Prepared according to the procedure described in EXAMPLE 22 using (4-fluoro-phenyl)-acetic acid in place of phenylactic acid. MS (ES+): m/z 346.05 [MH+].

### Example 25

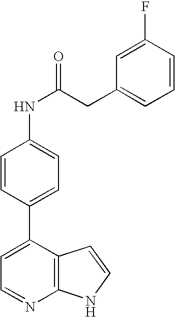

### 2-(3-Fluoro-phenyl)-N-[4-(1H-pyrrolo[2,3-b]pyridin-4-yl)-phenyl]-acetamide

Prepared according to the procedure described in EXAMPLE 22 using (3-fluoro-phenyl)-acetic acid in place of phenylactic acid. MS (ES+): m/z 346.05 [MH+].

### Example 26

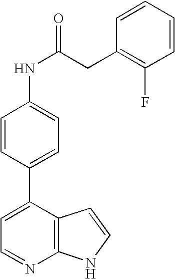

### 2-(2-Fluoro-phenyl)-N-[4-(1H-pyrrolo[2,3-b]pyridin-4-yl)-phenyl]-acetamide

Prepared according to the procedure described in EXAMPLE 22 using (2-fluoro-phenyl)-acetic acid in place of phenylactic acid. MS (ES+): m/z 345.99 [MH+].

### Example 27

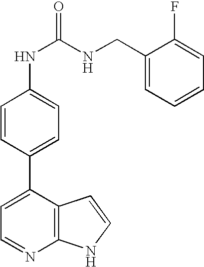

### 1-(2-Fluoro-benzyl)-3-[4-(1H-pyrrolo[2,3-b]pyridin-4-yl)-phenyl]-urea

To a solution of 4-(1H-pyrrolo[2,3-b]pyridin-4-yl)-phenylamine (52.25 mg, 0.25 mmole) in THF (3 mL) was added 2-fluorobenzyl isocyanate (37.78 mg, 0.25 mmole), the resulting mixture was stirred at rt overnight. The precipitate from the reaction mixture was collected by filtration and washed with isopropyl ether to afford the title compound. ¹H NMR (DMSO-d₆): δ 4.36 (d, 2H, J=5.7 Hz), 6.61 (m, 1H), 6.69 (t, 1H, J=6.0 Hz), 7.16 (m, 3H), 7.30 (m, 1H), 7.39 (m, 1H), 7.49 (t, 1H, J=3.0 Hz), 7.62 (m, 4H), 8.22 (d, 1H, J=5.1 Hz), 8.79 (s, 1H), 11. 69 (brs, 1H). MS (ES+): m/z 360.98 [MH+].

### Example 28

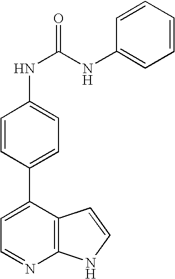

### 1-Phenyl-3-[4-(1H-pyrrolo[2,3-b]pyridin-4-yl)-phenyl]-urea

Prepared according to the procedure described in EXAMPLE 27 using phenyl isocyanate in place of 2-fluorobenzyl isocyanate. MS (ES+): m/z 329.01 [MH+].

### Example 29

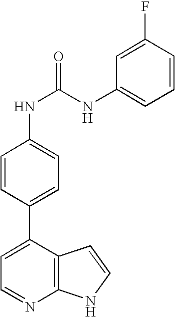

### 1-(3-Fluoro-phenyl)-3-[4-(1H-pyrrolo[2,3-b]pyridin-4-yl)-phenyl]-urea

Prepared according to the procedure described in EXAMPLE 27 using 3-fluorophenyl isocyanate in place of 2-fluorobenzyl isocyanate. MS (ES+): m/z 347.00 [MH+].

### Example 30

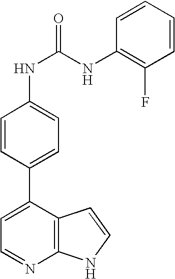

### 1-(2-Fluoro-phenyl)-3-[4-(1H-pyrrolo[2,3-b]pyridin-4-yl)-phenyl]-urea

Prepared according to the procedure described in EXAMPLE 27 using 2-fluorophenyl isocyanate in place of 2-fluorobenzyl isocyanate. MS (ES+): m/z 346.98 [MH+].

### Example 31

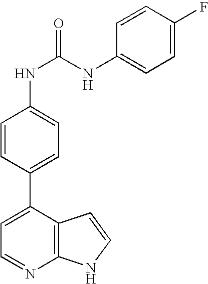

### 1-(4-Fluoro-phenyl)-3-[4-(1H-pyrrolo[2,3-b]pyridin-4-yl)-phenyl]-urea

Prepared according to the procedure described in EXAMPLE 27 using 4-fluorophenyl isocyanate in place of 2-fluorobenzyl isocyanate. MS (ES+): m/z 346.99 [MH+].

### Example 32

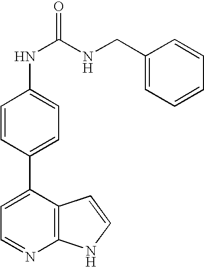

### 1-Benzyl-3-[4-(1H-pyrrolo[2,3-b]pyridin-4-yl)-phenyl]-urea

Prepared according to the procedure described in EXAMPLE 27 using benzyl isocyanate in place of 2-fluorobenzyl isocyanate. MS (ES+): m/z 343.01 [MH+].

### Example 33

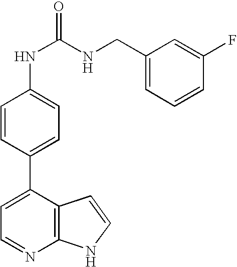

### 1-(3-Fluoro-benzyl)-3-[4-(1H-pyrrolo[2,3-b]pyridin-4-yl)-phenyl]-urea

Prepared according to the procedure described in EXAMPLE 27 using 3-fluorobenzyl isocyanate in place of 2-fluorobenzyl isocyanate. MS (ES+): m/z 360.99 [MH+].

### Example 34

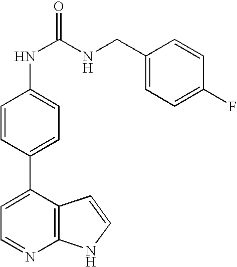

### 1-(4-Fluoro-benzyl)-3-[4-(1H-pyrrolo[2,3-b]pyridin-4-yl)-phenyl]-urea

Prepared according to the procedure described in EXAMPLE 27 using 4-fluorobenzyl isocyanate in place of 2-fluorobenzyl isocyanate. MS (ES+): m/z 360.97 [MH+].

### Example 35

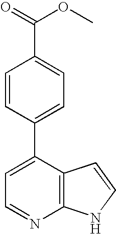

### 4-(1H-Pyrrolo[2,3-b]pyridin-4-yl)-benzoic acid methyl ester

A mixture of 4-chloro-7-azaindole (2.12 g, 14 mmole) in 1,4-dioxane (80mL) and water (20 mL) in a 250 mL, two-necked round bottomed flask was charged with K₂CO₃ (1.145 g, 9.3 mmole), 4-methoxycarbonylphenylboronic acid (2.9 g, 16.1 mmole), Pd(dppf)₂Cl₂.CH₂Cl₂ catalyst (551 mg, 0.67 mmole). Nitrogen was bubbled into the reaction mixture for 15 min at rt and then heated at 100° C. overnight under nitrogen atmosphere. The reaction mixture was cooled to rt and added triethylamine (3 mL) and evaporated to dryness and purified by column chromatography. The crude was taken in methylene chloride and loaded onto the column. The column was eluted with 15 to 35% ethyl acetate in methylene chloride and the desired fractions from column were collected. the resulting solid was triturated with hot isopropyl ether, cooled to rt and filtered to give the title compound . ¹H NMR (DMSO-d₆): δ 3.89 (s, 3H), 6.63 (m, 1H), 7.25 (d, 1H, J=4.2 Hz), 7.58 (t, 1H, J=3.0 Hz), 7.92 (d, 2H, J=7.8 Hz), 8.12 (d, 2H, J=8.4 Hz), 8.31 (d, 1H, J=5.1 Hz), 11. 96 (brs, 1H); MS (ES+): m/z 253.15 [MH+].

### Example 36

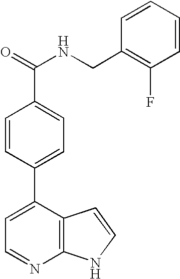

### N-(2-Fluoro-benzyl)-4-(1H-pyrrolo[2,3-b]pyridin-4-yl)-benzamide

a) To a solution of 4-(1H-pyrrolo[2,3-b]pyridin-4-yl)-benzoic acid methyl ester (1.8 g, 7.1 mmole) in a mixture of MeOH/THF (1:1, 30 mL) was added aqueous KOH solution (12%, 13 mL, 21.3 mmole), the resulting mixture was stirred at rt overnight. The reaction mixture was evaporated to dryness and charged with water (10 mL) and AcOH (1.5 mL), the resulting solid was collected by filtration and dried in the vacuum oven to give 4-(1H-pyrrolo[2,3-b]pyridin-4-yl)-benzoic acid as an off-white solid. ¹H NMR (DMSO-d₆): δ 6.63 (m, 1H), 7.23 (d, 1H, J=4.8 Hz), 7.56 (t, 1H, J=3 Hz), 7.85 (d, 2H, J=8.1 Hz), 8.09 (d, 2H, J=8.4 Hz), 8.30 (d, 1H, J=5.1 Hz), 11.85 (brs, 1H).

b) A solution of 2-fluorobenzylamine (37.5 mg, 0.3 mmole), EDC.HCl (69 mg, 0.36 mmole) and HOBt (40.5 mg, 0.3 mmole) in methylene chloride (5 mL) was charged with N,N-diisopropylethyl amine (77.6 mg, 0.6 mmole) and 4-(1H-pyrrolo[2,3-b]pyridin-4-yl)-benzoic acid (71.1 mg, 0.3 mmole). The resulting mixture was stirred at rt overnight, then evaporated to dryness and the resulted residue was triturated with water (10 mL). The solid was collected by filtration, washed with water, dried in vacuum oven to afford the title compound. ¹H NMR (DMSO-d₆): δ 4,55 (d, 2H, J=6 Hz), 6.62 (m, 1H), 7.17 (m, 3H), 7.29 (m, 2H), 7.56 (t, 1H, J=3 Hz), 7.86 (d, 2H, J=8.1 Hz), 8.06 (d, 2H, J=8.1 Hz), 8.30 (d, 1H, J=4.8 Hz), 9.13 (t, 1H), 11.83 (brs, 1H); MS (ES+): m/z 345.99 [MH+].

### Example 37

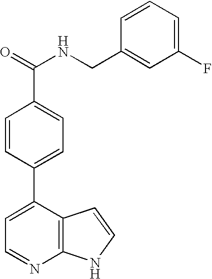

### N-(3-Fluoro-benzyl)-4-(1H-pyrrolo[2,3-b]pyridin-4-yl)-benzamide

Prepared according to the procedure described in EXAMPLE 36 using 3-fluorobenzylamine in place of 2-fluorobenzylamine. MS (ES+): m/z 345.99 [MH+].

### Example 38

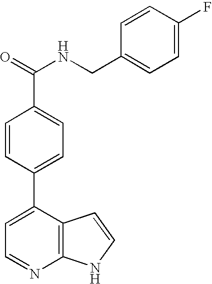

### N-(4-Fluoro-benzyl)-4-(1H-pyrrolo[2,3-b]pyridin-4-yl)-benzamide

Prepared according to the procedure described in EXAMPLE 36 using 4-fluorobenzylamine in place of 2-fluorobenzylamine. MS (ES+): m/z 345.98 [MH+].

### Example 39

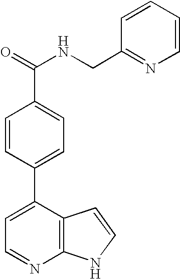

### N-Pyridin-2-ylmethyl-4-(1H-pyrrolo[2,3-b]pyridin-4-yl)-benzamide

Prepared according to the procedure described in EXAMPLE 36 using C-pyridin-2-yl-methylamine in place of 2-fluorobenzylamine. MS (ES+): m/z 329.22 [MH+].

### Example 40

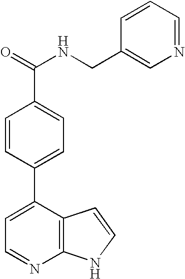

### N-Pyridin-3-ylmethyl-4-(1H-pyrrolo[2,3-b]pyridin-4-yl)-benzamide

Prepared according to the procedure described in EXAMPLE 36 using C-pyridin-3-yl-methylamine in place of 2-fluorobenzylamine. MS (ES+): m/z 329.22 [MH+].

### Example 41

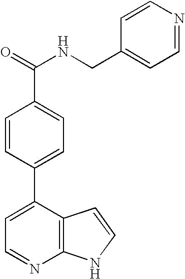

### N-Pyridin-4-ylmethyl-4-(1H-pyrrolo[2,3-b]pyridin-4-yl)-benzamide

Prepared according to the procedure described in EXAMPLE 36 using C-pyridin-4-yl-methylamine in place of 2-fluorobenzylamine. MS (ES+): m/z 329.22 [MH+].

### Example 42

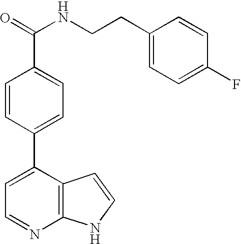

### N-[2-(4-Fluoro-phenyl)-ethyl]-4-(1H-pyrrolo[2,3-b]pyridin-4-yl)-benzamide

Prepared according to the procedure described in EXAMPLE 36 using 2-(4-fluoro-phenyl)-ethylamine in place of 2-fluorobenzylamine. MS (ES+): m/z 360.20 [MH+].

### Example 43

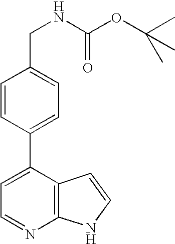

### [4-(1H-pyrrolo[2,3-b]pyridin-4-yl)-benzyl]-carbamic acid tert-butyl ester

A mixture of 4-chloro-7-azaindole (1.516 g, 10 mmole) in 1,4-dioxane (48 mL) and water (12 mL) in a 250 mL, two-necked round bottomed flask was charged with K₂CO₃ (0.820 g, 5.9 mmole), [4-(N—BOC-aminomethyl)phenylboronic acid (2.88 g, 11.5 mmole), Pd(dppf)₂Cl₂.CH₂Cl₂ catalyst (371 mg, 0.45 mmole). Nitrogen was bubbled into the reaction mixture for 15 min at rt and then heated at 100° C. overnight under nitrogen atmosphere. The reaction mixture was cooled to rt and added triethylamine (3 mL) and evaporated to dryness and purified by column chromatography. The crude was taken in methylene chloride and loaded onto the column. The column was eluted with 20-40% ethyl acetate in methylene chloride, the desired fractions from column were collected and the resulting solid was triturated with hot isopropyl ether, cooled to rt and filtered to give the title compound as a pale yellow solid. ¹H NMR (CDCl₃): δ 1.49 (s, 9H), 4.41 (d, 2H, J=6.3 Hz), 4.98 (brs, 1H), 6.79 (m, 1H), 7.17 (d, 1H, J=4.8 Hz), 7.39 (t, 1H, J=3.0 Hz), 7.44 (d, 2H, J=8.4 Hz), 7.73 (d, 2H, J=8.4 Hz), 8.37 (d, 1H, J=5.1 Hz), 10.01 (brs, 1H); MS (ES+): m/z 324.09 [MH+].

### Example 44

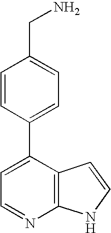

### 4-(1H-Pyrrolo[2,3-b]pyridin-4-yl)-benzylamine

To an ice cooled suspension of [4-(1H-pyrrolo[2,3-b]pyridin-4-yl)-benzyl]-carbamic acid tert-butyl ester (2 g, 6.18 mmole) in methylene chloride (50 mL) was added 8N HCl in 1,4-dioxane (8 mL, 64 mmole), the resulting mixture was stirred at rt overnight. The reaction mixture was then evaporated to dryness and diluted with diethyl ether (20 mL) The solid was collected by filtration and washed with ether (10 mL). The solid was then taken in water (10 mL) and basified with saturated aqueous NaHCO₃, the resulting solid was collected by filtration, washed with water (2×10 mL) and dried in vacuum oven over P₂O₅ to give the title compound as an off-white solid (1.1 g, 84%). ¹H NMR (DMSO-d₆): δ 3.97 (s, 2H), 6.58 (m, 1H), 7.17 (d, 1H, J=5.1 Hz), 7.53 (m, 1H), 7.58 (d, 2H, J=8.4 Hz), 7.76 (d, 2H, J=7.8 Hz), 8.27 (d, 1H, J=4.8 Hz), 11.80 (brs, 1H). MS (ES+): m/z 224.18 [MH+].

### Example 45

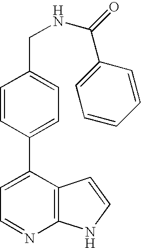

### N-[4-(1H-Pyrrolo[2,3-b]pyridin-4-yl)-benzyl]-benzamide

Prepared according to the procedure described in EXAMPLE 22 using 4-(1H-pyrrolo[2,3-b]pyridin-4-yl)-benzylamine and benzoic acid in place of 4-(1H-pyrrolo[2,3-b]pyridin-4-yl)-phenylamine and phenylactic acid. MS (ES+): m/z 327.99 [MH+].

### Example 46

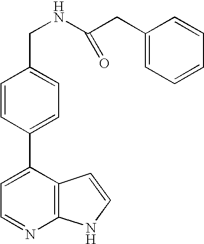

### 2-Phenyl-N-[4-(1H-pyrrolo[2,3-b]pyridin-4-yl)-benzyl]-acetamide

Prepared according to the procedure described in EXAMPLE 22 using 4-(1H-pyrrolo[2,3-b]pyridin-4-yl)-benzylamine in place of 4-(1H-pyrrolo[2,3-b]pyridin-4-yl)-phenylamine. MS (ES+): m/z 342.09 [MH+].

### Example 47

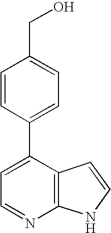

### [4-(1H-pyrrolo[2,3-b]pyridin-4-yl)-phenyl]-methanol

To a suspension of 4-(1H-pyrrolo[2,3-b]pyridin-4-yl)-benzaldehyde (100 mg, 0.45 mmole) in methylene chloride (5 mL) was added NaBH(OCOCH₃)₃ (21 0 mg, 0.98 mmole), the resulting mixture was heated under reflux for 3 h. The reaction mixture was evaporated to dryness, and taken in aqueous saturated sodium bicarbonate (10 mL), extracted with methylene chloride (2×10 mL). The organic layer washed with brine, dried over anhydrous Na₂SO₄, filtered and concentrated. The crude residue was purified on column chromatography using 2% methanol in methylene chloride as an eluant to give the title compound as a pale yellow solid. ¹H NMR (DMSO-d₆): δ 4.57 (d, 2H, J=5.4 Hz), 5.26 (t, 1H, J=5.7 Hz), 6.59 (m, 1H), 7.16 (d, 1H, J=5.1 Hz), 7.48 (d, 2H, J=8.1 Hz), 7.52 (t, 1H, J=3.0 Hz), 7.72 (d, 2H, J=8.1 Hz), 8.26 (d, 1H, J=4.8 Hz), 11.75 (brs, 1H). MS (ES+): m/z 225.13 [MH+].

### Example 48

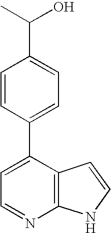

### 1-[4-(1H-Pyrrolo[2,3-b]pyridin-4-yl)-phenyl]-ethanol

Prepared according to the procedure described in EXAMPLE 47 using 1-[4-(1H-pyrrolo[2,3-b]pyridin-4-yl)-phenyl]-ethanone in place of 4-(1H-pyrrolo[2,3-b]pyridin-4-yl)-benzaldehyde. MS (ES+): m/z 239.05 [MH+].

### Example 49

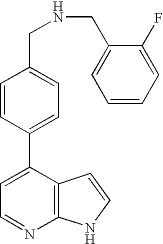

### (2-Fluoro-benzyl)-[4-(1H-pyrrolo[2,3-b]pyridin-4-yl)-benzyl]-amine

a) To a suspension of 4-(1H-pyrrolo[2,3-b]pyridin-4-yl)-benzaldehyde (111 mg, 0.5 mmole) in THF (10mL) was added 2-fluorobenzylamine (125 mg, 1 mmole) and the mixture was stirred at rt overnight. Then NaBH(OCOCH₃)₃ (211.9 mg, 1 mmole) was added as a solid and the mixture was stirred at rt overnight. The reaction mixture was then diluted with ethyl acetate (15 mL) and washed with saturated sodium bicarbonate, dried over Na₂SO₄, filtered and concentrated. The crude residue was purified by column chromatography. The column was packed with methylene chloride and the compound was loaded in methylene chloride. It was then eluted with 40-50% ethyl acetate in methylene chloride. The desired fractions from column were collected and then triturated with hot isopropyl ether, cooled and filtered to give the title compound as a white solid. ¹H NMR (CDCl₃): δ 3.97 (s, 2H), 4.00 (s, 2H), 6.79 (m, 1H), 7.13-7.33 (m, 4H), 7.51(m, 2H), 7.58 (d, 2H, J=8.7 Hz), 7.80 (d, 2H, J=4.5 Hz), 8.45 (d, 1H, J=4.8 Hz), 10.22 (brs, 1H). MS (ES+): m/z 332.20 [MH+].

b) A mixture of 4-chloro-7-azaindole (5.32 g, 35 mmole) in 1,4-dioxane (200 mL) and water (50 mL) in a 500 mL, two-necked round bottomed flask was charged with K₂CO₃ (9.6 g, 70 mmole), 4-formylphenylboronic acid (6.32 g, 42 mmole), Pd(dppf)₂Cl₂.CH₂Cl₂ catalyst (1.36 g, 1.66 mmole). Nitrogen was bubbled into the reaction mixture for 15 min at rt and then heated at 100° C. overnight under nitrogen atmosphere. The reaction mixture was cooled to rt, evaporated to dryness and the residue was treated with water (100 mL) and the resulting solid was collected by filtration. It was then purified by column chromatography using 0.5% methanol in CH₂Cl₂ as eluant. The desired fractions from column were collected, evaporated and the resulting solid was triturated with hot isopropyl ether, cooled to rt and filtered to give the title compound as a pale yellow solid. ¹H NMR (DMSO-d₆): δ 6.64 (m, 1H), 7.26 (d, 1H, J=5.1 Hz), 7.59 (m, 1H), 7.99 (d, 2H, J=8.1 Hz), 8.07 (d, 2H, J=8.1 Hz), 8.32 (d, 1H, J=4.8 Hz), 10.09 (s, 1H), 11.88 (brs, 1H).

### Example 50

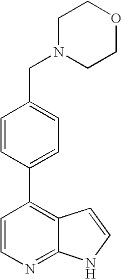

### 4-(4-Morpholin-4-ylmethyl-phenyl)-1H-pyrrolo[2,3-b]pyridine

Prepared according to the procedure described in EXAMPLE 49 using morpholine in place of 2-fluorobenzylamine. ¹H NMR (400 MHz, CDCl₃) δ: 10.23-10.43 (m, 1H), 8.35-8.46 (m, 1H), 7.69-7.78 (m, 2H), 7.47-7.55 (m, 2H), 7.39-7.44 (m, 1H), 7.18-7.23 (m, 1H), 6.68-6.77 (m, 1H), 3.71-3.87 (m, 4H), 3.58-3.69 (m, 2H), 2.44-2.69 (m, 4H). MS (ES+): m/z 294.17 [MH⁺].

### Example 51

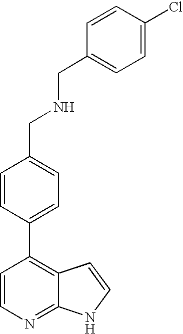

### (4-Chloro-benzyl)-[4-(1H-pyrrolo[2,3-b]pyridin-4-yl)-benzyl]-amine

Prepared according to the procedure described in EXAMPLE 49 using 4-chlorobenzylamine in place of 2-fluorobenzylamine. ¹H NMR (400 MHz, CDCl₃) δ: 8.09 (d, 1H, J=5.1 Hz), 7.59 (d, 2H, J=8.1 Hz), 7.33 (d, 2H, J=8.1 Hz), 7.23 (d, 1H, J=3.5 Hz), 7.13-7.20 (m, 4H), 7.02 (d, 1H, J=5.1 Hz), 6.52 (d, 1H, J=3.5 Hz), 3.72 (s, 2H), 3.67(s, 2H). MS (ES+): m/z 348.08/350.10 (3/1) [MH⁺].

### Example 52

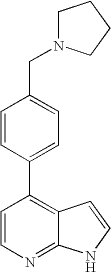

### 4-(4-pyrrolidin-1-ylmethyl-phenyl)-1H-pyrrolo[2,3-b]pyridine

Prepared according to the procedure described in EXAMPLE 49 using pyrrolidine in place of 2-fluorobenzylamine. ¹H NMR (400 MHz, CD₃OD) δ: 8.27 (d, 1H, J=5.1 Hz), 7.83 (d, 2H, J=8.3 Hz), 7.62 (d, 2H, J=8.1 Hz), 7.47 (d, 1H, J=3.5 Hz), 7.23 (d, 1H, J=5.1 Hz), 6.69 (d, 1H, J=3.5 Hz), 4.08 (s, 2H), 2.99 (s, 4H), 2.00 (t, 4H, J=3.3 Hz). MS (ES+): m/z 278.20 [MH⁺].

### Example 53

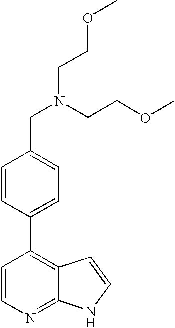

### Bis-(2-methoxy-ethyl)-[4-(1H-pyrrolo[2,3-b]pyridin-4-yl)-benzyl]-amine

Prepared according to the procedure described in EXAMPLE 49 using bis-(2-methoxy-ethyl)-amine in place of 2-fluorobenzylamine. ¹H NMR (400 MHz, CDCl₃) δ: 9.26 (s, 1H), 8.38 (d, 1H, J=5.1 Hz), 7.76 (t, 2H, J=8.3 Hz), 7.59 (m, 2H), 7.39 (d, 1H, J=3.5 Hz), 7.16-7.21 (m, 1H), 6.69-6.74 (m, 1H), 4.05 (s, 2H), 3.71 (s, 4H), 3.35-3.40 (m, 6H), 3.01 (s, 4H). MS (ES+): m/z 340.19 [MH⁺].

### Example 54

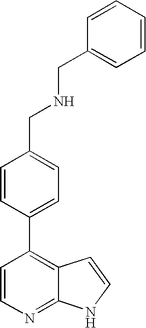

### Benzyl-[4-(1H-pyrrolo[2,3-b]pyridin-4-yl)-benzyl]-amine

Prepared according to the procedure described in EXAMPLE 49 using benzylamine in place of 2-fluorobenzylamine. ¹H NMR (400 MHz, CD₃OD) δ: 8.47 (s, 1H), 8.26 (d, 1H, J=5.1 Hz), 7.80-7.92 (m, 2H), 7.62 (d, 2H, J=8.3 Hz), 7.39-7.52 (m, 5H), 7.21 (d, 1H, J=5.1 Hz), 6.65 (d, 1H, J=3.8 Hz), 4.22 (s, 2H), 4.18 (s, 2H). MS (ES+): m/z 314.18 [MH⁺].

### Example 55

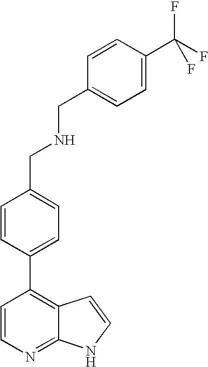

### [4-(1H-Pyrrolo[2,3-b]pyridin-4-yl)-benzyl]-(4-trifluoromethyl-benzyl)-amine

Prepared according to the procedure described in EXAMPLE 49 using 4-trifluoromethyl-benzylamine in place of 2-fluorobenzylamine. ¹H NMR (400 MHz, CDCl₃) δ: 8.25 (s, 1H), 7.59 (d, 2H, J=8.1 Hz), 7.44-7.51 (m, 4H), 7.35 (d, 1H, J=3.5 Hz), 7.14 (d, 1H, J=4.6 Hz), 6.65 (d, 1H, J=3.5 Hz), 3.78-4.00 (m, 2H), 3.27-3.42 (m, 2H). MS (ES+): m/z 382.10 [MH⁺].

### Example 56

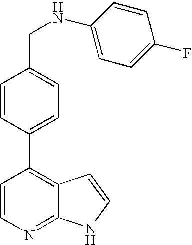

### (4-Fluoro-phenyl)-[4-(1H-pyrrolo[2,3-b]pyridin-4-yl)-benzyl]-amine

Prepared according to the procedure described in EXAMPLE 49 using 4-fluoroaniline in place of 2-fluorobenzylamine. ¹H NMR (400 MHz, CD₃OD) δ: 8.42 (d, 1H, J=5.8 Hz), 7.83-7.94 (m, 2H), 7.57-7.76 (m, 4H), 6.90-7.05 (m, 3H), 6.77-6.89 (m, 2H), 4.52 (s, 2H). MS (ES+): m/z 318.13 [MH⁺].

### Example 57

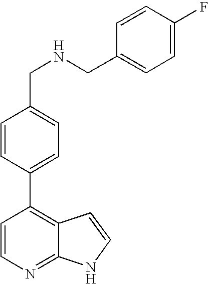

### (4-Fluoro-benzyl)-[4-(1H-pyrrolo[2,3-b]pyridin-4-yl)-benzyl]-amine

Prepared according to the procedure described in EXAMPLE 49 using 4-fluorobenzylamine in place of 2-fluorobenzylamine. ¹H NMR (400 MHz, CD₃OD) δ: 8.43 (s, 1H), 7.97 (d, 2H, J=8.3 Hz), 7.75 (d, 2H, J=8.3 Hz), 7.64 (d, 1H, J=3.5 Hz), 7.55-7.62 (m, 2H), 7.48 (d, 1H, J=5.3 Hz), 7.20-7.30 (m, 2H), 6.85 (d, 1H, J=3.5 Hz), 4.39 (s, 2H), 4.34 (s, 2H). MS (ES+): m/z 332.10 [MH⁺].

### Example 58

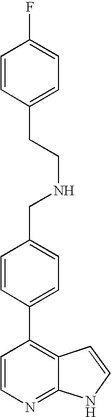

### [2-(4-Fluoro-phenyl)-ethyl]-[4-(1H-pyrrolo[2,3-b]pyridin-4-yl)-benzyl]-amine

Prepared according to the procedure described in EXAMPLE 49 using 2-(4-fluoro-phenyl)-ethylamine in place of 2-fluorobenzylamine. ¹H NMR (400 MHz, CD₃OD) δ: 8.41 (s, 1H), 7.96 (d, 2H, J=8.3 Hz) 7.74 (d, 2H, J=8.3 Hz), 7.65 (d, 1H, J=3.8 Hz), 7.49 (d, 1, J=5.6 Hz), 7.27-7.36 (m, 2H) 7.05-7.14 (m, 2H), 6.85 (d, 1H, J=3.5 Hz), 4.37 (s, 2H), 3.34-3.38 (m, 2H), 2.96-3.12 (m, 2H). MS (ES+): m/z 346.12 [MH⁺].

### Example 59

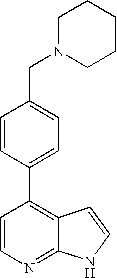

### 4-(4-Piperidin-1-ylmethyl-phenyl)-1H-pyrrolo[2,3-b]pyridine

Prepared according to the procedure described in Example 49 using piperidine in place of 2-fluorobenzylamine. ¹H NMR (400 MHz, CD₃OD) δ: 8.51 (s, 1H), 8.27 (d, 1H, J=5.1 Hz), 7.85-7.91 (m, 2H), 7.66 (d, 2H, J=8.3 Hz), 7.48 (d, 1H, J=3.8 Hz), 7.23 (d, 1H, J=5.1 Hz), 6.67 (d, 1H, J=3.5 Hz), 4.27 (s, 2H), 3.16 (s, 4H), 1.78-1.93 (m, 4H), 1.66 (s, 2H). MS (ES+): m/z 292.21 [MH⁺].

### Example 60

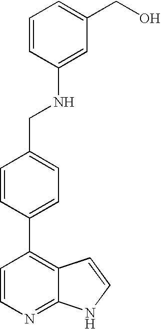

### {3-[4-(1H-Pyrrolo[2,3-b]pyridin-4-yl)-benzylamino]-phenyl}-methanol

Prepared according to the procedure described in EXAMPLE 49 using (3-amino-phenyl)-methanol in place of 2-fluorobenzylamine. ¹H NMR (400 MHz, CD₃OD) δ: 8.23 (d, 1H, J=5.3 Hz), 7.73 (d, 2H, J=8.3 Hz), 7.56 (d, 2H, J=8.3 Hz), 7.43 (d, 1H, J=3.8 Hz), 7.20 (d, 1H, J=5.1 Hz), 7.08 (t, 1H, J=7.7 Hz), 6.71 (d, 1H, J=1.8 Hz), 6.68 (d, 1H, J=3.5 Hz), 6.56-6.65 (m, 2H), 4.50 (s, 2H), 4.44 (s, 2H). MS (ES+): m/z 330.15 [MH⁺].

### Example 61

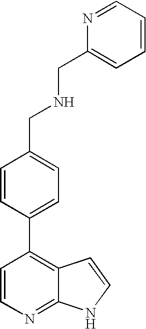

### Pyridin-2-ylmethyl-[4-(1H-pyrrolo[2,3-b]pyridin-4-yl)-benzyl]-amine

Prepared according to the procedure described in EXAMPLE 49 using C-pyridin-2-yl-methylamine in place of 2-fluorobenzylamine. ¹H NMR (400 MHz, CD₃OD) δ: 8.61-8.67 (m, 1H), 8.48 (s, 1H), 8.26 (d, 1H, J=5.1 Hz), 7.83-7.87 (m, 3H), 7.66 (d, 2H, J=8.3 Hz), 7.44-7.51 (m, 2H), 7.36-7.43 (m, 1H), 7.22 (d, 1H, 5.1 Hz), 6.67 (d, 2H, J=8.3 Hz), 4.33 (s, 2H), 4.30 (s, 2H). MS (ES+): m/z 315.16 [MH⁺].

### Example 62

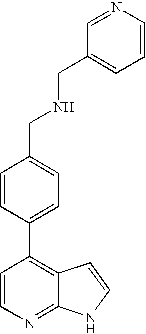

### Pyridin-3-ylmethyl-[4-(1H-pyrrolo[2,3-b]pyridin-4-yl)-benzyl]-amine

Prepared according to the procedure described in EXAMPLE 49 using C-pyridin-3-yl-methylamine in place of 2-fluorobenzylamine. ¹H NMR (400 MHz, CD₃OD) δ: 8.65 (d, 1H, J=1.8 Hz), 8.56 (dd, 1H, J=5.1, 1.5 Hz), 8.44 (s, 1H), 8.25 (d, 1H, J=5.1 Hz), 7.95-8.00 (m, 1H), 7.84 (d, 2H, J=8.3 Hz), 7.62 (d, 2H, J=8.1 Hz), 7.50 (dd, 1H, J=7.2, 4.9 Hz), 7.47 (d, 1H, J=3.5 Hz), 7.22 (d, 1H, J=5.3 Hz), 6.66 (d, 1H, J=3.5 Hz), 4.18 (s, 2H), 4.17 (s, 2H). MS (ES+): m/z 315.19 [MH⁺].

### Example 63

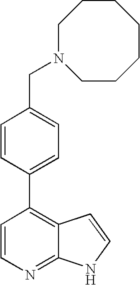

### 4-(4-Azocan-1-ylmethyl-phenyl)-1H-pyrrolo[2,3-b]pyridine

Prepared according to the procedure described in EXAMPLE 49 using azocane in place of 2-fluorobenzylamine. ¹H NMR (400 MHz, CD₃OD) δ: 8.40 (d, 1H, J=5.8 Hz), 7.93-8.00 (m, 2H), 7.77 (d, 2H, J=8.3 Hz), 7.65 (d, 1H, J=3.5 Hz), 7.50 (d, 1H, J=5.8 Hz), 6.85 (d, 1H, J=3.8 Hz), 4.48 (s, 1H), 3.45-3.61 (m, 2H), 2.08 (br, 2H), 1.59-1.94 (m, 10H). MS (ES+): m/z 320.24 [MH⁺].

### Example 64

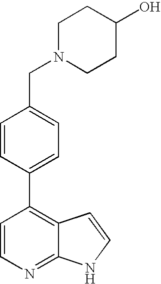

### 1-[4-(1H-Pyrrolo[2,3-b]pyridin-4-yl)-benzyl]-piperidin-4-ol

Prepared according to the procedure described in EXAMPLE 49 using piperidin-4-ol in place of 2-fluorobenzylamine. ¹H NMR (400 MHz, CD₃OD) δ: 8.29 (s, 1H), 7.89 (d, 2H, J=8.1 Hz), 7.68 (d, 2H, J=8.3 Hz), 7.48 (d, 1H, J=3.5 Hz), 7.24 (d, 1H, J=4.6 Hz), 6.68 (d, 1H, J=3.5 Hz), 4.37 (s, 2H), 3.98 (br, 2H), 3.44 (br, 2H), 3.16-3.26 (br, 2H), 2.06 (br, 2H), 1.86 (br, 2H). MS (ES+): m/z 308.18 [MH⁺].

### Example 65

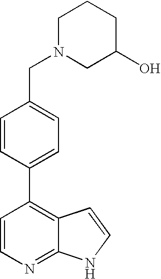

### 1-[4-(1H-Pyrrolo[2,3-b]pyridin-4-yl)-benzyl]-piperidin-3-ol

Prepared according to the procedure described in EXAMPLE 49 using piperidin-3-ol in place of 2-fluorobenzylamine. ¹H NMR (400 MHz, CD₃OD) δ: 8.27 (d, 1H, J=5.1 Hz), 7.88 (d, 2H, J=8.4 Hz), 7.65 (d, 2H, J=8.1 Hz), 7.48 (d, 1H, J=3.5 Hz), 7.23 (d, 1H, J=5.1 Hz), 6.67 (d, 1H, J=3.5 Hz), 4.58 (br, 2H), 4.13-4.36 (m, 2H), 3.98 (br, 1H), 3.35-3.79 (m, 1H), 3.05-3.20 (m, 2H), 2.06-2.22 (m, 2H), 1.70-1.95 (m, 2H), 1.62 (br, 1H). MS (ES+): m/z 308.18 [MH⁺].

### Example 66

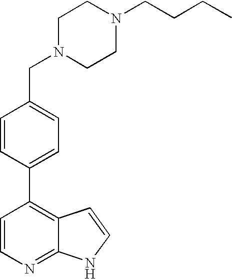

### 4-[4-(4-Butyl-piperazin-1-ylmethyl)-phenyl]-1H-pyrrolo[2,3-b]pyridine

Prepared according to the procedure described in EXAMPLE 49 using 1-butyl-piperazine in place of 2-fluorobenzylamine. ¹H NMR (400 MHz, CD₃OD) δ: 8.26 (br, 1H), 7.78 (d, 2H, J=8.3 Hz), 7.54 (d, 2H, J=8.1 Hz), 7.45 (d, 2H, J=3.8 Hz), 7.20 (d, 1H, J=4.8 Hz), 6.66 (d, 1H, J=3.5 Hz), 3.76 (s, 2H), 2.53-3.70 (br, 7H), 3.01-3.13 (m, 3H), 1.63-1.79 (m, 2H), 1.34-1.47 (m, 2H), 1.00 (t, 3H, J=7.3 Hz). MS (ES+): m/z 349.22 [MH⁺].

### Example 67

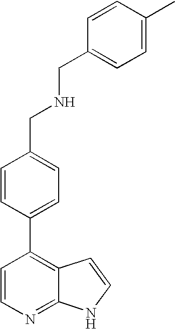

### (4-Methyl-benzyl)-[4-(1H-pyrrolo[2,3-b]pyridin-4-yl)-benzyl]-amine

Prepared according to the procedure described in EXAMPLE 49 using 4-methyl-benzylamine in place of 2-fluorobenzylamine. ¹H NMR (400 MHz, CD₃OD) δ: 8.38 (br, 1H), 8.23 (br, 1H), 7.78 (d, 1H, J=8.3 Hz), 7.53 (s, 1H), 7.51 (s, 1H), 7.39 (d, 1H, J=3.5 Hz), 7.25-7.30 (m, 2H), 7.19-7.23 (m, 2H), 7.16 (d, 1H, J=5.1 Hz), 6.62 (d, 1H, J=3.5 Hz), 4.04 (s, 2H), 3.99 (s, 2H), 2.35 (s, 3H). MS (ES+): m/z 328.22 [MH⁺].

### Example 68

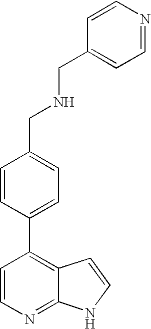

### Pyridin-4-ylmethyl-[4-(1H-pyrrolo[2,3-b]pyridin-4-yl)-benzyl]-amine

Prepared according to the procedure described in EXAMPLE 49 using C-pyridin-4-yl-methylamine in place of 2-fluorobenzylamine. ¹H NMR (400 MHz, CD₃OD) δ: 8.52 (d, 2H, J=5.8 Hz), 8.26-8.35 (br, 1H), 8.23 (d, 1H, J=5.1 Hz), 7.77 (d, 2H, J=8.1 Hz), 7.55 (d, 2H, J=8.3 Hz), 7.47 (d, 2H, J=6.1 Hz), 7.41 (d, 1H, J=3.5 Hz), 7.17 (d, 1H, J=5.1 Hz), 6.64 (d, 1H, J=3.5 Hz), 4.03 (m, 4H). MS (ES+): m/z 315.20 [MH⁺].

### Example 69

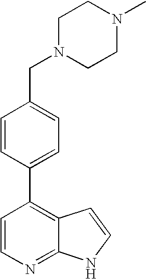

### 4-[4-(4-Methyl-piperazin-1-ylmethyl)-phenyl]-1H-pyrrolo[2,3-b]pyridine

Prepared according to the procedure described in EXAMPLE 49 using 1-methyl-piperazine in place of 2-fluorobenzylamine. ¹H NMR (400 MHz, CD₃OD) δ: 8.50 (br, 1H), 8.24 (br, 1H), 7.76 (d, 2H, J=8.3 Hz), 7.53 (d, 2H, J=8.3 Hz), 7.45 (d, 1H, J=3.8 Hz), 7.20 (d, 1H, J=5.1 Hz), 6.66 (d, 1H, J=3.5 Hz), 3.72 (s, 2H), 3.11 (br, 4H), 2.73 (s, 3H), 2.65-2.84 (br, 4H). MS (ES+): m/z 307.24 [MH⁺].

### Example 70

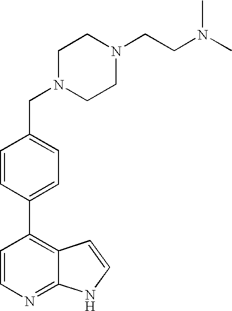

### Dimethyl-(2-{4-[4-(1H-pyrrolo[2,3-b]pyridin-4-yl)-benzyl]-piperazin-1-yl}-ethyl)-amine

Prepared according to the procedure described in EXAMPLE 49 using dimethyl-(2-piperazin-1-yl-ethyl)-amine in place of 2-fluorobenzylamine. ¹H NMR (400 MHz, CD₃OD) δ: 8.47 (br, 1H), 8.25 (d, 1H, J=5.1 Hz), 7.79 (d, 2H, J=8.1 Hz), 7.56 (d, 2H, J=8.1 Hz), 7.46 (d, 1H, J=3.5 Hz), 7.21 (d, 1H, J=5.1 Hz), 6.67 (d, 1H, J=3.5 Hz), 3.85 (s, 2H), 3.22 (m, 2h), 2.86 (s, 6H), 2.64-2.83 (br, 10H). MS (ES+): m/z 364.27 [MH⁺].

### Example 71

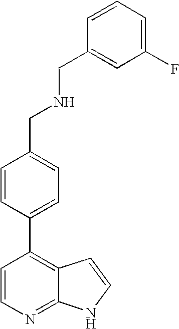

### (3-Fluoro-benzyl)-[4-(1H-pyrrolo[2,3-b]pyridin-4-yl)-benzyl]-amine

Prepared according to the procedure described in EXAMPLE 49 using 3-fluorobenzylamine in place of 2-fluorobenzylamine. ¹H NMR (400 MHz, CD₃OD) δ: 8.22 (d, 2H, J=4.8 Hz), 7.71-7.76 (m, 2H), 7.49 (d, 2H, J=8.3 Hz), 7.29-7.37 (m, 2H), 7.08-7.18 (m, 3H), 6.97-7.03 (m, 1H), 3.97 (s, 2H), 3.93 (s, 2H). MS (ES+): m/z 332.20 [MH⁺].

### Example 72

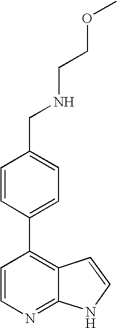

### (2-Methoxy-ethyl)-[4-(1H-pyrrolo[2,3-b]pyridin-4-yl)-benzyl]-amine

Prepared according to the procedure described in EXAMPLE 49 using 2-methoxy-ethylamine in place of 2-fluorobenzylamine. ¹H NMR (400 MHz, CD₃OD) δ: 8.46 (br, 1H), 8.25 (br, 1H), 7.80 (d, 2H, J=8.3 Hz), 7.58 (d, 2H, J=8.3 Hz), 7.40 (d, 1H, J=3.5 Hz), 7.16 (d, 1H, J=5.1 Hz), 6.62 (d, 1H, J=3.5 Hz), 4.19 (s, 2H), 3.64 (m, 2H), 3.39 (s, 3H), 3.12 (m, 2H). MS (ES+): m/z 282.22 [MH⁺.

### Example 73

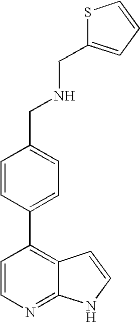

### [4-(1H-Pyrrolo[2,3-b]pyridin-4-yl)-benzyl]-thiophen-2-ylmethyl-amine

Prepared according to the procedure described in EXAMPLE 49 using C-thiophen-2-yl-methylamine in place of 2-fluorobenzylamine. ¹H NMR (400 MHz, CD₃OD) δ: 8.25 (d, 1H, J=5.1 Hz), 7.80-7.85 (m, 2H), 7.59 (d, 2H, J=8.3 Hz), 7.44-7.48 m, 2H), 7.22 (d, 1H, J=5.1 Hz), 7.19 (d, 1H, J=3.3 Hz), 7.07 (dd, 1H, J=5.2, 3.4 Hz), 6.67 (d, 1H, J=3.5 Hz), 4.29 (s, 2H), 4.11 (s, 2H). MS (ES+): m/z 320.18 [MH⁺].

### Example 74

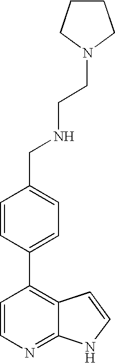

### (2-Pyrrolidin-1-yl-ethyl)-[4-(1H-pyrrolo[2,3-b]pyridin-4-yl)-benzyl]-amine

Prepared according to the procedure described in EXAMPLE 49 using 2-pyrrolidin-1-yl-ethylamine in place of 2-fluorobenzylamine. ¹H NMR (400 MHz, CD₃OD) δ: 8.54 (br, 1H), 8.22-8.28 (m, 1H), 7.76-7.82 (m, 2H), 7.58 (d, 2H, J=8.3 Hz), 7.43-7.47 (m, 1H), 7.18-7.22 (m, 1H), 6.63-6.68 (m, 1H), 4.01 (s, 2H), 3.09-3.20 (m, 6H), 3.01 (t, 2H, J=6.1 Hz), 1.95-2.06 (m, 4H). MS (ES+): m/z 321.26 [MH⁺].

### Example 75

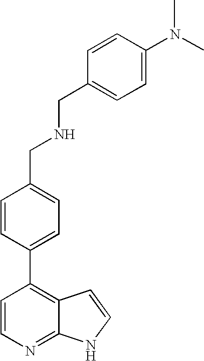

### Dimethyl-(4-{[4-(1H-pyrrolo[2,3-b]pyridin-4-yl)-benzylamino]-methyl}-phenyl)-amine

Prepared according to the procedure described in EXAMPLE 49 using (4-aminomethyl-phenyl)-dimethylamine in place of 2-fluorobenzylamine. ¹H NMR (400 MHz, CD₃OD) δ: 8.55 (s, 1H), 8.26 (d, 1H, J=5.1 Hz), 7.84 (d, 2H, J=8.3 Hz), 7.60 (d, 2H, J=8.4 Hz), 7.46 (d, 1H, J=3.5 Hz), 7.29 (d, 2H, J=8.8 Hz), 7.22 (d, 1H, J=5.1 Hz), 6.80 (d, 2H, J=8.8 Hz), 6.66 (d, 1H, J=3.5 Hz), 4.13 (s, 2H), 4.02 (s, 2H), 2.96 (s, 6H). MS (ES+): m/z 357.29 [MH⁺].

### Example 76

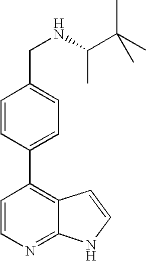

### (S)-[4-(1H-Pyrrolo[2,3-b]pyridin-4-yl)-benzyl]-(1,2,2-trimethyl-propyl)-amine

Prepared according to the procedure described in EXAMPLE 49 using (S)-1,2,2-trimethyl-propylamine in place of 2-fluorobenzylamine. ¹H NMR (400 MHz, CD₃OD/CDCl₃) δ: 8.28 (br, 1H), 8.08 (d, 1H, J=5.1 Hz), 7.64 (d, 2H, J=8.1 Hz), 7.41 (d, 2H, J=8.1 Hz), 7.23 (d, 1H, J=3.5 Hz), 7.00 (d, 1H, J=5.1 Hz), 6.47 (d, 1H, J=3.5 Hz), 4.21 (d, 1H, J=13.6 Hz), 3.8 (d, 1H, J=13.6 Hz), 2.46 (q, 1H, J=6.82 Hz), 1.08 (d, 3H, J=6.6 Hz), 0.76 (s, 9H). MS (ES+): m/z 308.92 [MH⁺].

### Example 77

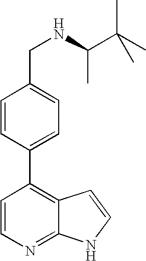

### (R)-[4-(1H-Pyrrolo[2,3-b]pyridin-4-yl)-benzyl]-(1,2,2-trimethyl-propyl)-amine

Prepared according to the procedure described in EXAMPLE 49 using (R)-1,2,2-trimethyl-propylamine in place of 2-fluorobenzylamine. ¹H NMR (400 MHz, CD₃OD) δ: 8.52 (br, 1H), 8.27 (d, 1H, J=5.1 Hz), 7.89 (d, 2H, J=8.3 Hz), 7.70 (d, 2H, J=8.3 Hz), 7.48 (d, 1H, J=3.5 Hz), 7.24 (d, 1H, J=5.1 Hz), 6.66 (d, 1H, J=3.5 Hz), 4.45 (d, 1H, J=13.6 Hz), 4.28 (d, 1H, J=13.6 Hz), 2.90 (q, 1H, J=6.7 Hz), 1.34 (d, 3H, J=6.8 Hz), 0.99 (s, 9H). MS (ES+): m/z 308.92 [MH+].

### Example 78

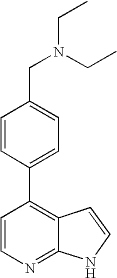

### Diethyl-[4-(1H-pyrrolo[2,3-b]pyridin-4-yl)-benzyl]-amine

Prepared according to the procedure described in EXAMPLE 49 using diethylamine in place of 2-fluorobenzylamine. MS (ES+): m/z 280.24 [MH+].

### Example 79

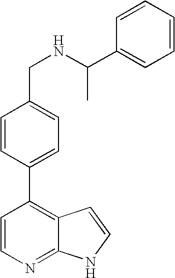

### (1-Phenyl-ethyl)-[4-(1H-pyrrolo[2,3-b]pyridin-4-yl)-benzyl]-amine

Prepared according to the procedure described in EXAMPLE 49 using 1-phenylethylamine in place of 2-fluorobenzylamine. MS (ES+): m/z 328.20 [MH+].

### Example 80

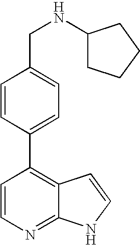

### Cyclopentyl-[4-(1H-pyrrolo[2,3-b]pyridin-4-yl)-benzyl]-amine

Prepared according to the procedure described in EXAMPLE 49 using cyclopentylamine in place of 2-fluorobenzylamine. MS (ES+): m/z 292.23 [MH+].

### Example 81

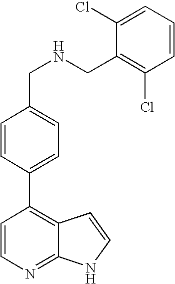

### (2,6-Dichloro-benzyl)-[4-(1H-pyrrolo[2,3-b]pyridin-4-yl)-benzyl]-amine

Prepared according to the procedure described in EXAMPLE 49 using 2,6-dichloro-benzylamine in place of 2-fluorobenzylamine. MS (ES+): m/z 382.05/384.07 (9/6) [MH+].

### Example 82

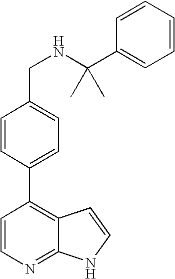

### (1-Methyl-1-phenyl-ethyl)-[4-(1H-pyrrolo[2,3-b]pyridin-4-yl)-benzyl]-amine

Prepared according to the procedure described in EXAMPLE 49 using 1-methyl-1-phenylethylamine in place of 2-fluorobenzylamine. MS (ES+): m/z 342.15 [MH+].

### Example 83

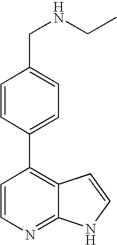

### Ethyl-[4-(1H-pyrrolo[2,3-b]pyridin-4-yl)-benzyl]-amine

Prepared according to the procedure described in EXAMPLE 49 using ethylamine in place of 2-fluorobenzylamine. MS (ES+): m/z 252.16 [MH+].

### Example 84

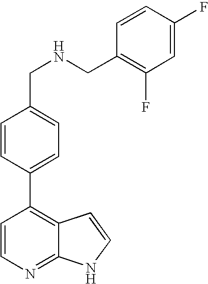

### (2,4-Difluoro-benzyl)-[4-(1H-pyrrolo[2,3-b]pyridin-4-yl)-benzyl]-amine

Prepared according to the procedure described in EXAMPLE 49 using 2,4-difluoro-benzylamine in place of 2-fluorobenzylamine. MS (ES+): m/z 350.03 [MH+].

### Example 85

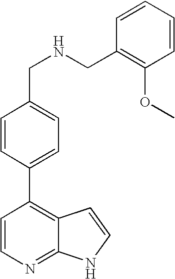

### (2-Methoxy-benzyl)-[4-(1H-pyrrolo[2,3-b]pyridin-4-yl)-benzyl]-amine

Prepared according to the procedure described in EXAMPLE 49 using 2-methoxy-benzylamine in place of 2-fluorobenzylamine. MS (ES+): m/z 344.08 [MH+].

### Example 86

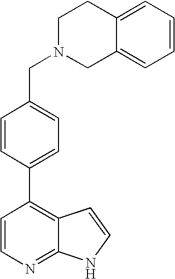

### 2-[4-(1H-Pyrrolo[2,3-b]pyridin-4-yl)-benzyl]-1,2,3,4-tetrahydro-isoquinoline

Prepared according to the procedure described in EXAMPLE 49 using 1,2,3,4-tetrahydro-isoquinoline in place of 2-fluorobenzylamine. MS (ES+): m/z 340.06 [MH+].

### Example 87

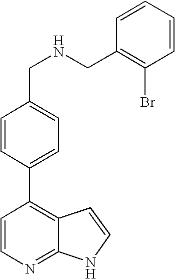

### (2-Bromo-benzyl)-[4-(1H-pyrrolo[2,3-b]pyridin-4-yl)-benzyl]-amine

Prepared according to the procedure described in EXAMPLE 49 using 2-bromobenzylamine in place of 2-fluorobenzylamine. MS (ES+): m/z 391.95/393.95 (1/1) [MH+].

### Example 88

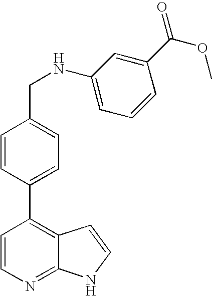

### 3-[4-(1H-Pyrrolo[2,3-b]pyridin-4-yl)-benzylamino]-benzoic acid methyl ester

Prepared according to the procedure described in EXAMPLE 49 using 3-amino-benzoic acid methyl ester in place of 2-fluorobenzylamine. MS (ES+): m/z 357.98 [MH+].

### Example 89

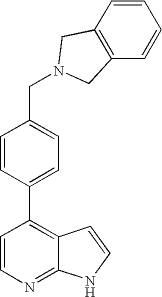

### 4-[4-(1,3-Dihydro-isoindol-2-ylmethyl)-phenyl]-1H-pyrrolo[2,3-b]pyridine

Prepared according to the procedure described in EXAMPLE 49 using 2,3-dihydro-1H-isoindole in place of 2-fluorobenzylamine. MS (ES+): m/z 326.04 [MH+].

### Example 90

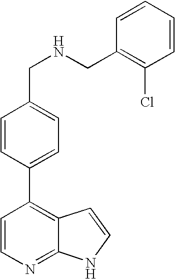

### (2-Chloro-benzyl)-[4-(1H-pyrrolo[2,3-b]pyridin-4-yl)-benzyl]-amine

Prepared according to the procedure described in EXAMPLE 49 using 2-chlorobenzylamine in place of 2-fluorobenzylamine. MS (ES+): m/z 347.95/349.94 (3/1) [MH+].

### Example 91

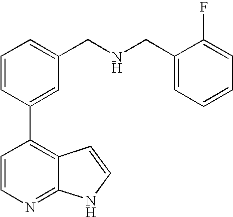

### (2-Fluoro-benzyl)-[3-(1H-pyrrolo[2,3-b]pyridin-4-yl)-benzyl]-amine

Prepared according to the procedure described in EXAMPLE 49 using 3-formylphenylboronic acid in place of 4-formylphenylboronic acid. MS (ES+): m/z 331.99 [MH+].

### Example 92

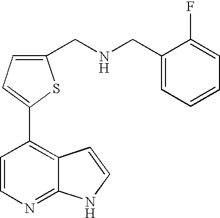

### (2-Fluoro-benzyl)-[5-(1H-pyrrolo[2,3-b]pyridin-4-yl)-thiophen-2-ylmethyl]-amine

Prepared according to the procedure described in EXAMPLE 49 using 5-formyl-thiophene-2-boronic acid in place of 4-formylphenylboronic acid. MS (ES+): m/z 337.97 [MH+].

### Example 93

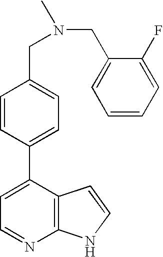

### (2-Fluoro-benzyl)-methyl-[4-(1H-pyrrolo[2,3-b]pyridin-4-yl)-benzyl]-amine

Prepared according to the procedure described in EXAMPLE 49 using (2-fluoro-benzyl)-methylamine in place of 2-fluorobenzylamine. MS (ES+): m/z 328.01 [MH+].

### Example 94

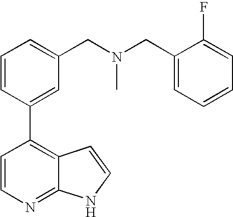

### (2-Fluoro-benzyl)-methyl-[3-(1H-pyrrolo[2,3-b]pyridin-4-yl)-benzyl]-amine

Prepared according to the procedure described in EXAMPLE 49 using 3-formylphenylboronic acid and (2-fluoro-benzyl)-methylamine in place of 4-formylphenylboronic acid and 2-fluorobenzylamine. MS (ES+): m/z 327.92 [MH+].

The following examples were prepared according to the procedures described herewithin:

| EX. # | Structure | Name |
|---|---|---|
| 95 | 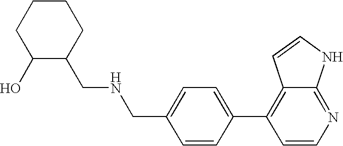 | 2-{[4-(1H-Pyrrolo[2,3-b]pyridin-4-yl)- benzylamino]-methyl}-cyclohexanol |
| 96 | 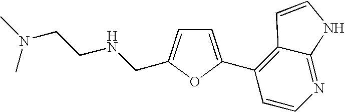 | N,N-Dimethyl-N′-[5-(1H-pyrrolo[2,3- b]pyridin-4-yl)-furan-2-ylmethyl]-ethane-1,2- diamine |
| 97 | 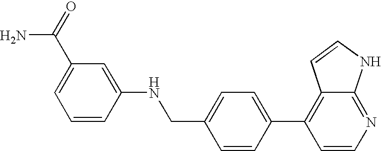 | 3-[4-(1H-Pyrrolo[2,3-]pyridin-4-yl)- benzylamino]-benzamide |
| 98 | 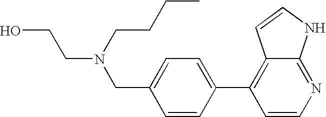 | 2-{Butyl-[4-(1H-pyrrolo[2,3-b]pyridin-4-yl)- benzyl]-amino}-ethanol |
| 99 | 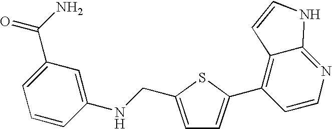 | 3-{[5-(1H-Pyrrolo[2,3-b]pyridin-4-yl)- thiophen-2-ylmethyl]-amino}-benzamide |
| 100 | 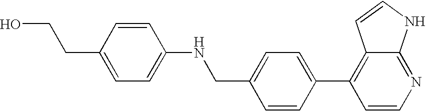 | 2-{4-[4-(1H-Pyrrolo[2,3-b]pyridin-4-yl)- benzylamino]-phenyl-ethanol |
| 101 | 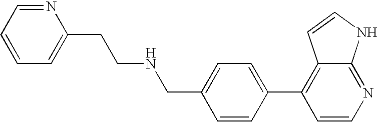 | (2-Pyridin-2-yl-ethyl)-[4-(1H-pyrrolo[2,3- b]pyridin-4-yl)-benzyl]-amine |
| 102 | 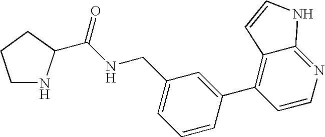 | Pyrrolidine-2-carboxylic acid 3-(1H- pyrrolo[2,3-b]pyridin-4-yl)-benzylamide |
| 103 | 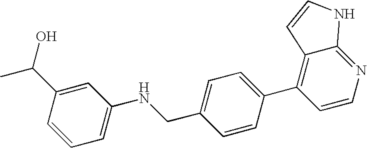 | 1-{3-[4-(1H-Pyrrolo[2,3-b]pyridin-4-yl)- benzylamino]-phenyl-ethanol |
| 104 | 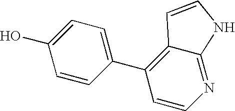 | 4-(1H-Pyrrolo[2,3-b]pyridin-4-yl)-phenol |
| 105 | 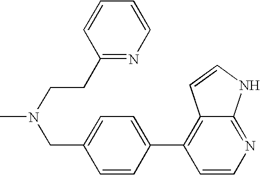 | Methyl-(2-pyridin-2-yl-ethyl)-[4-(1H- pyrrolo[2,3-b]pyridin-4-yl)-benzyl]-amine |
| 106 | 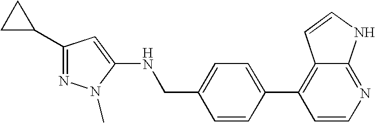 | (5-Cyclopropyl-2-methyl-2H-pyrazol-3-yl)-[4- (1H-pyrrolo[2,3-b]pyridin-4-yl)-benzyl]-amine |
| 107 | 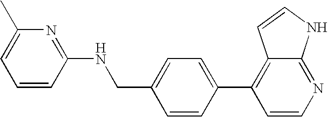 | (6-Methyl-pyridin-2-yl)-[4-(1H-pyrrolo[2,3- b]pyridin-4-yl)-benzyl]-amine |
| 108 | 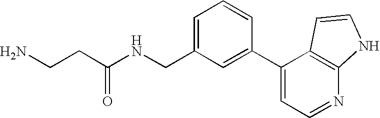 | 3-Amino-N-[3-(1H-pyrrolo[2,3-b]pyridin-4- yl)-benzyl]-propionamide |
| 109 | 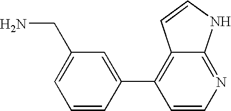 | 3-(1H-Pyrrolo[2,3-b]pyridin-4-yl)-benzylamine |
| 110 | 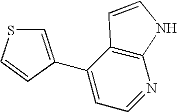 | 4-Thiophen-3-yl-1H-pyrrolo[2,3-b]pyridine |
| 111 | 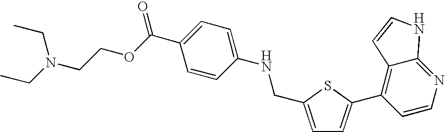 | 4-{[5-(1H-Pyrrolo[2,3-b]pyridm-4-yl)- thiophen-2-ylmethyl]-amino}-benzoic acid 2- diethylamino-ethyl ester |
| 112 | 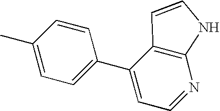 | 4-p-Tolyl-1H-pyrrolo[2,3-b]pyridine |
| 113 | 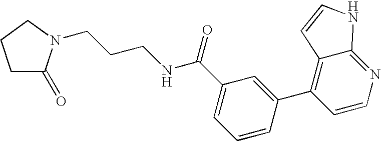 | N-[3-(2-Oxo-pyrrolidin-1-yl)-propyl]-3-(1H- pyrrolo[2,3-b]pyridin-4-yl)- benzamide |
| 114 | 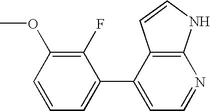 | 4-(2-Fluoro-3-methoxy-phenyl)-1H- pyrrolo[2,3-b]pyridine |
| 115 | 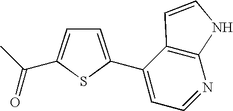 | 1-[5-(1H-Pyrrolo[2,3-b]pyridin-4-yl)-thiophen- 2-yl]-ethanone |
| 116 | 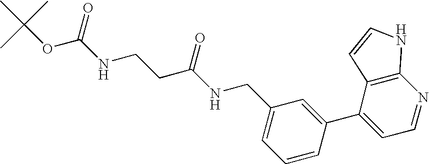 | {2-[3-(1H-Pyrrolo[2,3-b]pyridin-4-yl)- benzylcarbamoyl]-ethyl}-carbamic acid tert- butyl ester |
| 117 | 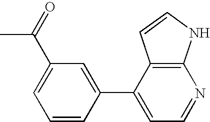 | 1-[3-(1H-Pyrrolo[2,3-b]pyridin-4-yl)-phenyl]- ethanone |
| 118 | 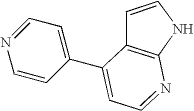 | 4-Pyridin-4-yl-1H-pyrrolo[2,3-b]pyridine |
| 119 | 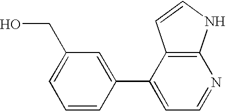 | [3-(1H-Pyrrolo[2,3-b]pyridin-4-yl)-phenyl]- methanol |
| 120 | 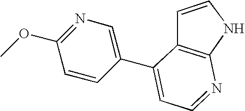 | 4-(6-Methoxy-pyridin-3-yl)-1H-pyrrolo[2,3- b]pyridine |
| 121 | 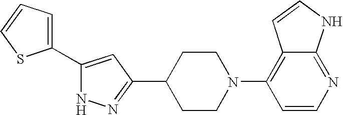 | 4-[4-(5-Thiophen-2-yl-1H-pyrazol-3-yl)- piperidine-1-yl]-1H-pyrrolo[2,3-b]pyridine |
| 122 | 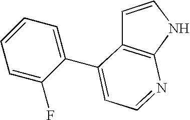 | 4-(2-Fluoro-phenyl)-1H-pyrrolo[2,3-b]pyridine |
| 123 | 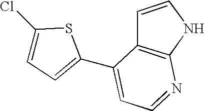 | 4-(5-Chloro-thiophen-2-yl)-1H-pyrrolo[2,3- b]pyridine |
| 124 | 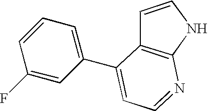 | 4-(3-Fluoro-phenyl)-1H-pyrrolo[2,3-b]pyridine |
| 125 | 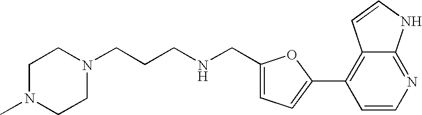 | [3-(4-Methyl-piperazin-1-yl)-propyl]-[5-(1H- pyrrolo[2,3-b]pyridin-4-yl)-furan-2-ylmethyl]- amine |
| 126 | 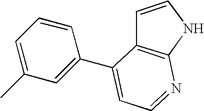 | 4-m-Tolyl-1H-pyrrolo[2,3-b]pyridine |
| 127 | 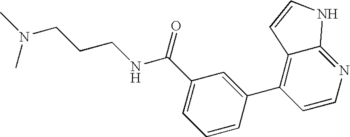 | N-(3-Dimethylamino-propyl)-3-(1H- pyrrolo[2,3-b]pyridin-4-yl)-benzamide |
| 128 | 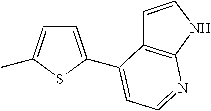 | 4-(5-Methyl-thiophen-2-yl)-1H-pyrrolo[2,3- b]pyridine |
| 129 | 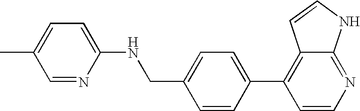 | (5-Methyl-pyridin-2-yl)-[4-(1H-pyrrolo[2,3- b]pyridin-4-yl)-benzyl]-amine |
| 130 | 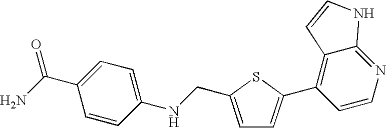 | 4-{[5-(1H-Pyrrolo[2,3-b]pyridin-4-yl)- thiophen-2-ylmethyl]-amino}-benzamide |
| 131 | 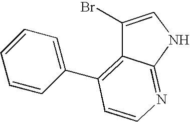 | 3-Bromo-4-phenyl-1H-pyrrolo[2,3-b]pyridine |
| 132 | 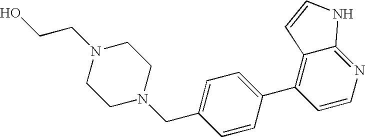 | 2-{4-[4-(1H-Pyrrolo[2,3-b]pyridin-4-yl)- benzyl]-piperazin-1-yl}-ethanol |
| 133 | 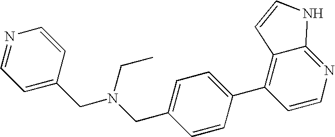 | Ethyl-pyridin-4-ylmethyl-[4-(1H-pyrrolo[2,3- b]pyridin-4-yl)-benzyl]-amine |
| 134 | 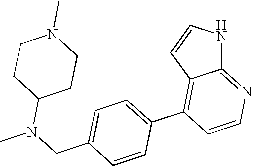 | Methyl-(1-methyl-piperidin-4-yl)-[4-(1H- pyrrolo[2,3-b]pyridin-4-yl)-benzyl]-amine |
| 135 | 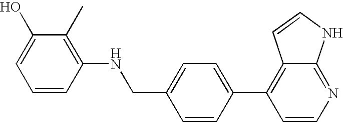 | 2-Methyl-3-[4-(1H-pyrrolo[2,3-b]pyridin-4-yl)- benzylamino]-phenol |
| 136 | 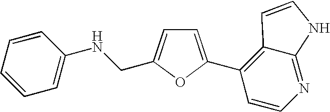 | Phenyl-[5-(1H-pyrrolo[2,3-b]pyridin-4-yl)- furan-2-ylmethyl]-amine |
| 137 | 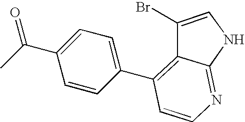 | 1-[4-(3-Bromo-1H-pyrrolo[2,3-b]pyridin-4-yl)- phenyl]-ethanone |
| 138 | 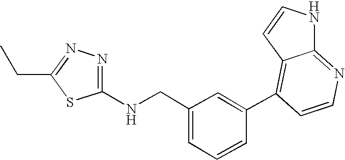 | (5-Ethyl-[1,3,4]thiadiazol-2-yl)-[3-(1H- pyrrolo[2,3-b]pyridin-4-yl)-benzyl]-amine |
| 139 | 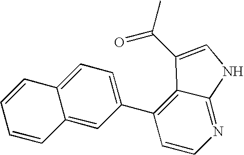 | 1-(4-Naphthalen-2-yl-1H-pyrrolo[2,3- b]pyridin-3-yl)-ethanone |
| 140 | 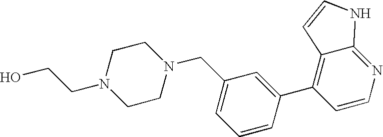 | 2-{4-[3-(1H-Pyrrolo[2,3-b]pyridin-4-yl)- benzyl]-piperazin-1-yl}-ethanol |
| 141 | 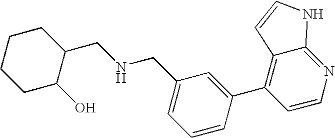 | 2-{[3-(1H-Pyrrolo[2,3-b]pyridin-4-yl)- benzylamino]-methyl}-cyclohexanol |
| 142 | 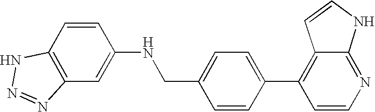 | (1H-Benzotriazol-5-yl)-[4-(1H-pyrrolo[2,3- b]pyridin-4-yl)-benzyl]-amine |
| 143 | 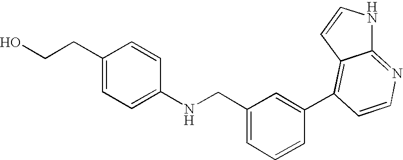 | 2-{4-[3-(1H-Pyrrolo[2,3-b]pyridin-4-yl)- benzylamino]-phenyl}-ethanol |
| 144 | 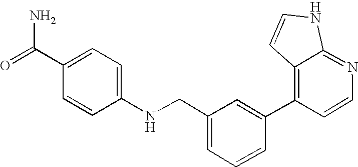 | 4-[3-(1H-Pyrrolo[2,3-b]pyridin-4-yl)- benzylamino]-benzamide |
| 145 | 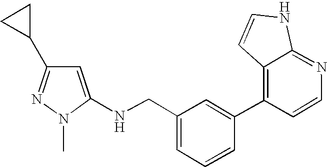 | (5-Cyclopropyl-2-methyl-2H-pyrazol-3-yl)-[3- (1H-pyrrolo[2,3-b]pyridin-4-yl)-benzyl]-amine |
| 146 | 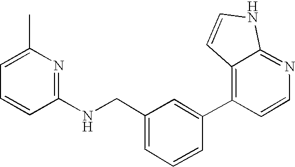 | (6-Methyl-pyridin-2-yl)-[3-(1H-pyrrolo[2,3- b]pyridin-4-yl)-benzyl]-amine |
| 147 | 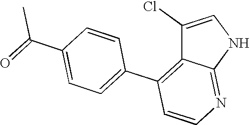 | 1-[4-(3-Chloro-1H-pyrrolo[2,3-b]pyridin-4-yl)- phenyl]-ethanone |
| 148 | 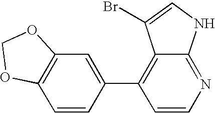 | 4-Benzo[1,3]dioxol-5-yl-3-bromo-1H- pyrrolo[2,3-b]pyridine |
| 149 | 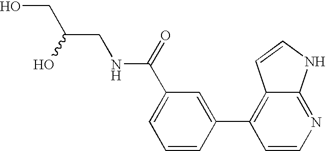 | N-(2,3-Dihydroxy-propyl)-3-(1H-pyrrolo[2,3- b]pyridin-4-yl)-benzamide |
| 150 | 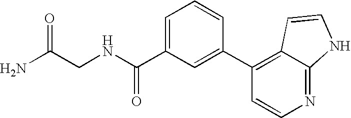 | N-Carbamoylmethyl-3-(1H-pyrrolo[2,3- b]pyridin-4-yl)-benzamide |
| 151 | 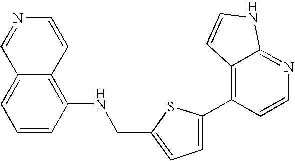 | Isoquinolin-5-yl-[5-(1H-pyrrolo[2,3-b]pyridin- 4-yl)-thiophen-2-ylmethyl]-amine |
| 152 | 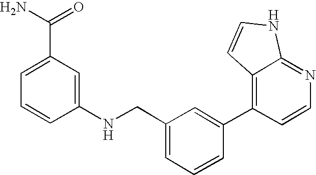 | 3-[3-(1H-Pyrrolo[2,3-b]pyridin-4-yl)- benzylamino]-benzamide |
| 153 | 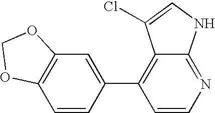 | 4-Benzo[1,3]dioxol-5-yl-3-chloro-1H- pyrrolo[2,3-b]pyridine |
| 154 | 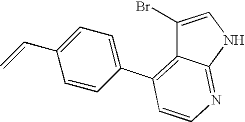 | 3-Bromo-4-(4-vinyl-phenyl)-1H-pyrrolo[2,3- b]pyridine |
| 155 | 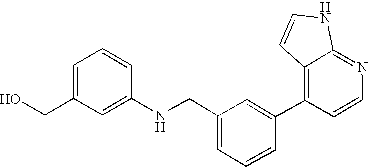 | {3-[3-(1H-Pyrrolo[2,3-b]pyridin-4-yl)- benzylamino]-phenyl}-methanol |
| 156 | 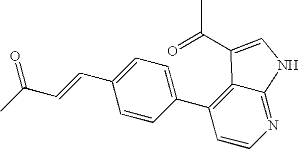 | (E)-4-[4-(3-Acetyl-1H-pyrrolo[2,3-b]pyridin-4- yl)-phenyl]-but-3-en-2-one |
| 157 | 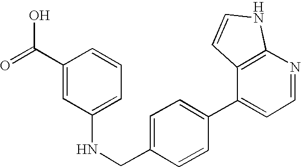 | 3-[4-(1H-Pyrrolo[2,3-b]pyridin-4-yl)- benzylamino]-benzoic acid |
| 158 | 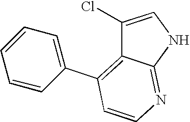 | 3-Chloro-4-phenyl-1H-pyrrolo[2,3-b]pyridine |
| 159 | 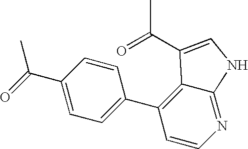 | 1-[4-(4-Acetyl-phenyl)-1H-pyrrolo[2,3- b]pyridin-3-yl]-ethanone |
| 160 | 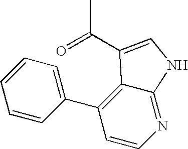 | 1-(4-Phenyl-1H-pyrrolo[2,3-b]pyridin-3-yl)- ethanone |
| 161 | 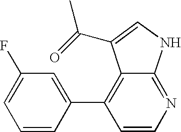 | 1-[4-(3-Fluoro-phenyl)-1H-pyrrolo[2,3- b]pyridin-3-yl]-ethanone |
| 162 | 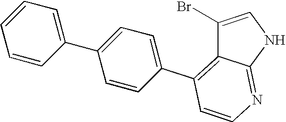 | 4-Biphenyl-4-yl-3-bromo-1H-pyrrolo[2,3- b]pyridine |
| 163 | 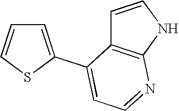 | 4-Thiophen-2-yl-1H-pyrrolo[2,3-b]pyridine |
| 164 | 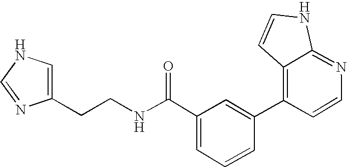 | N-[2-(1H-Imidazol-4-yl)-ethyl]-3-(1H- pyrrolo[2,3-b]pyridin-4-yl)-benzamide |
| 165 | 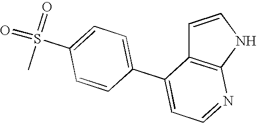 | 4-(4-Methanesulfonyl-phenyl)-1H-pyrrolo[2,3- b]pyridine |
| 166 | 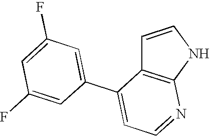 | 4-(3,5-Difluoro-phenyl)-1H-pyrrolo[2,3- b]pyridine |
| 167 | 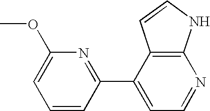 | 4-(6-Methoxy-pyridin-2-yl)-1H-pyrrolo[2,3- b]pyridine |
| 168 | 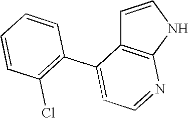 | 4-(2-Chloro-phenyl)-1H-pyrrolo[2,3-b]pyridine |
| 169 | 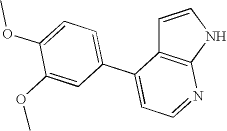 | 4-(3,4-Dimethoxy-phenyl)-1H-pyrrolo[2,3- b]pyridine |
| 170 | 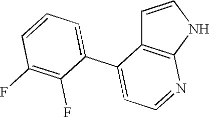 | 4-(2,3-Difluoro-phenyl)-1H-pyrrolo[2,3- b]pyridine |
| 171 | 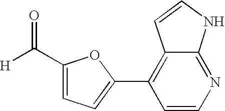 | 5-(1H-Pyrrolo[2,3-b]pyridin-4-yl)-furan-2- carbaldehyde |
| 172 | 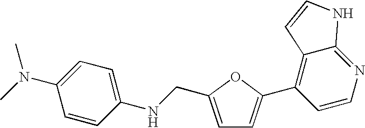 | N,N-Dimethyl-N′-[5-(1H-pyrrolo[2,3- b]pyridin-4-yl)-furan-2-ylmethyl]-benzene-1,4- diamine |
| 173 | 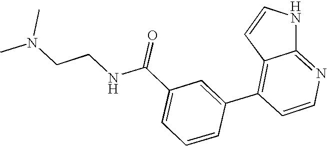 | N-(2-Dimethylamino-ethyl)-3-(1H-pyrrolo[2,3- b]pyridin-4-yl)-benzamide |
| 174 | 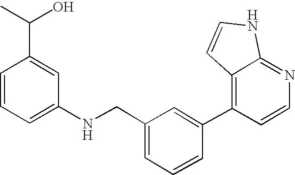 | 1-{3-[3-(1H-Pyrrolo[2,3-b]pyridin-4-yl)- benzylamino]-phenyl}-ethanol |
| 175 | 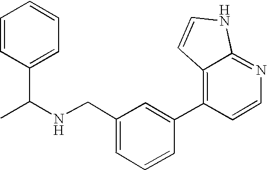 | (1-Phenyl-ethyl)-[3-(1H-pyrrolo[2,3-b]pyridin- 4-yl)-benzyl]-amine |
| 176 | 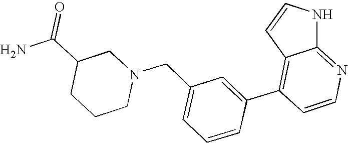 | 1-[3-(1H-Pyrrolo[2,3-b]pyridin-4-yl)-benzyl]- piperidine-3-carboxylic acid amide |

### 1H-Pyrrolo[2,3-b]pyridine 7-oxide m-chlorobenzoic acid salt

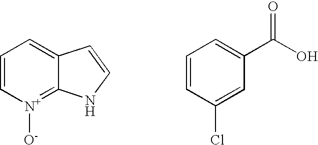

7-Azaindole (10.0 g, 84.5 mmol) was dissolved in 320 mL of diethyl ether. 3-chloroperoxybenzoic acid (26.2 g, 70% wt/wt, 152.1 mmol) was added portion-wise over 20 min. The reaction mixture was stirred at rt for 4 h. The resulting precipitate was collected by filtration to yield the title compound as a light yellow solid. ¹H NMR (400 MHz, DMSO-d₆): δ 12.44 (b. s., 1H), 8.12 (d, 1H, J=5.2 Hz), 7.87-7.89 (m, 2H), 7.70 (d, 1H, J=8.0 Hz), 7.63 (d, 1H, J=8.0 Hz), 7.53 (dd, 1H, J=8.0, 8.0 Hz), 7.44 (d, 1H, J=3.2 Hz), 7.06 (dd, 1H, J=8.0, 6.4 Hz), 6.57 (dd, 1H, J=3.6 Hz).

### 1H-pyrrolo[2,3-b]pyridine 7-oxide

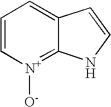

1H-pyrrolo[2,3-b]pyridine 7-oxide m-chlorobenzoic acid salt (24.0 g) was suspended in H₂O (50 mL) and charged with sat. aq. K₂CO₃ to pH=9. The reaction mixture turned green and white precipitate formed. The mixture was cooled with ice-bath for 2 h. The solid was collected by filtration and dried. ¹H NMR (400 MHz, DMSO-d₆): δ 12.47 (br. s., 1H), 8.12 (d, 1H, J=6.1 Hz), 7.63 (d, 1H, J=7.8 Hz), 7.45 (d, 1H, J=3.3 Hz), 7.05 (dd, 1H, J=8.0, 6.2 Hz), 6.57 (d, 1H, J=3.3 Hz).

### 4-Chloro-7-azaindole

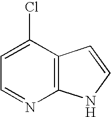

1H-pyrrolo[2,3-b]pyridine 7-oxide (4.70 g) was slowly added to cooled POCl₃ (42 mL) in portions. The resulting mixture was gently refluxed for 5 h. After cooled to rt, the POCl₃ was removed under reduced pressure. 40 mL of water was added to the cooled mixture (0° C.) and the mixture was basified with sat. aq. K₂CO₃. The precipitate was collected by filtration, washed with water and dried to give the title compound. ¹H NMR (400 MHz, DMSO-d₆): δ 12.03 (br. s., 1H), 8.17 (d, 1H, J=5.1 Hz), 7.59 (d, 1H, J=3.3 Hz), 7.19 (d, 1H, J=5.3 Hz), 6.50 (d, 1H, J=3.0 Hz).

### 4-Iodo-1H-pyrrolo[2,3-b]pyridine

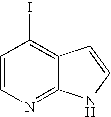

4-Chloro-7-azaindole (1.26 g, 8.25 mmol) was dissolved in dry acetonitrile (25 mL) in a 100 mL round bottom flask fitted with a condenser. Sodium iodide (1.96 g, 13.1 mmol) and acetyl chloride (1.37 g, 17.4 mmol) were then added and the reaction was put under N₂ atmosphere and the reaction was heated at reflux until complete (˜48h). The reaction was then concentrated in vacuo. A 10% solution of K₂CO₃ (10 mL) was then added and extracted with CH₂Cl₂ three times. The combined organic extracts were washed with 10% sodium sulfite, brine, dried over MgSO₄, and concentrated in vacuo. The crude product was purified using column chromatography (100% hexanes→hexanes:EtOAc =90:10) to yield 1-(4-iodo-pyrrolo[2,3-b]pyridin-1-yl)-ethanone. 1-(4-iodo-pyrrolo[2,3-b]pyridin-1-yl)-ethanone was then dissolved in 15 mL of THF. Sodium hydroxide (1M, 10 mL) was then added and the reaction stirred for 2.5h. The reaction was concentrated in vacuo and then partitioned between CH₂Cl₂ (40 mL) and water (20mL). The organic layer washed with brine, dried over MgSO₄, and concentrated in vacuo to yield the title compound as a white solid. MS (ES+): m/z 245 [MH⁺].

### 4-(5,5-Dimethyl-[1,3,2]dioxaborinan-2-yl)-1H-pyrrolo[2,3-b]pyridine

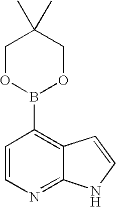

4-Iodo-7-Azaindole (1.00 g, 4.12 mmol), bis(neopentylglycolato)diboron (1.49 g, 6.59 mmol), potassium acetate (0.65 g, 6.59 mmol), and 1,1′-bis(diphenylphosphino)ferrocene dichloro palladium (II) dichloromethane complex (0.09 g, 0.12 mmol) were added to a round bottom flask. The flask was evacuated and backfilled with N₂ (3×). Anhydrous ethanol (20 mL) was added and the mixture was heated to reflux for 20 h. After cooling to rt, the reaction mixture was diluted with diethyl ether (35 mL) and then filtered through celite. The resulting filtrate was concentrated in vacuo and dissolved in ethyl acetate (50 mL). The solution washed with water (11 mL), brine (15 mL), and dried over MgSO₄. The filtrate was concentrated to a brown solid which was recrystallized with ethyl acetate to yield the title compound as a tan solid. The mother liquor was concentrated in vacuo and purified by column chromatography (Hexanes:EtOAc=80:20→60:40) to yield the title compound. ¹H NMR (400 MHz, DMSO-d₆) δ 0.99 (s, 6H), 3.83 (s, 4H), 6.69 (dd, 1H, J=1.8, 1.0 Hz), 7.30 (d, 1H, J=2.4 Hz), 7.45 (dd, 1H, J=2.8, 2.4 Hz), 8.18 (d, 1H, J=2.2 Hz), 11.52 (bs, 1H).

What is claimed is:

1. A compound of Formula I: 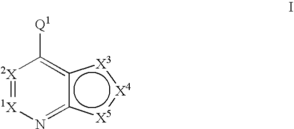
or a pharmaceutically acceptable salt thereof, wherein:
X¹ or X² are each independently N or —C(E¹)-;
X³, X⁴ and X⁵ are each independently N, O, S, —C(E¹)-, or ═C(E¹)-;
provided that
X³ is O or S when X⁴ and X⁵ are combined to equal —C(E^{1a})=C(E¹)-;
X⁵ is NH, O, or S when X³ and X⁴ are combined to equal —C(E^{1a})=C(E¹)-;
X⁵ is NH when X³ and X⁴ are combined to equal —N═C(E¹)-;
X⁵ is NH when X³ and X⁴ are combined to equal —C(E¹)=N—;
Q¹ is C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, heterobicycloC₅₋₁₀alkyl, spiroalkyl, or heterospiroalkyl; or -(Z¹)ₙ-(Y¹)ₘ—R¹; any of which is optionally substituted by one or more independent G¹ substituents;
E¹, E^{1a}, and G¹ are, in each instance, each independently equal to halo, —CF₃, —OCF₃, —OR²—NR²R³(R⁴)ⱼ₁, —C(═O)R², —CO₂R², —CONR²R³, —NO₂, —CN, —S(O)ⱼR², —SO₂NR²R³—NR²C(═O)R³—NR²C(═O)OR³, —NR²C(═O)NR³R⁴, —NR²S(O)ⱼ,R³, —C(═S)OR², —C(═O)SR², —NR²C(═NR³)NR⁴R⁵, —NR²C(═NR³)OR⁴, —NR²C(═NR³)SR⁴, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, oxo, —CF₃, —OCF₃, —OR²², NR²²R³³(R^{22a})ⱼ₁ₐ, —C(═O)R²², —CO₂R²², —C(═O)NR²²R³³, —NO₂, —CN, —S(═O)ⱼ₁ₐR²², —SO₂NR²²R³³, —NR²²C(═O)R³³, —NR²²C(═O)OR³³, —NR²²C(═O)NR³³R^{22a}, —NR²²S(O)ⱼ₁ₐR²², —C(═S)OR²², —C(═O)SR²², —NR²²C(═NR³³)NR^{22a}R^{33a}, —NR²²C(═NR³³)OR^{22a}, —NR²²C(═NR³³)SR^{22a}, —OC(═O)OR²², —OC(═O)NR²²R³³, —OC(═O)SR²², —SC(═O)OR²², or —SC(═O)NR²²R³³ substituents;
Z¹ is cycloC₃₋₈alkyl, heterocyclyl-C₀₋₁₀alkyl, aryl-C₀₋₁₀alkyl, hetaryl-C₀₋₁₀alkyl, heterobicycloC₅₋₁₀alkyl, spiroalkyl, or heterospiroalkyl, any of which is optionally substituted by one or more independent G¹ substituents;
Y¹ is —O—, —NR⁶—, —S(O)ⱼ₂—, —CR^{6a}R^{7a}—, —N(C(O)OR⁶)—, —N(C(O)R⁶)—, —N(SO₂R⁶)—, —(CR^{6a}R^{7a})O—, —(CR^{6a}R^{7a})S—, —(CR^{6a}R^{7a})N(R⁶)—, —CR^{6a}(NR⁶)—, —(CR^{6a}R^{7a})N(C(O)R⁶)—, —(CR^{6a}R^{7a})N(C(O)OR⁶)—, —(CR^{6a}R^{7a})N(SO₂R⁶)—, —(CR^{6a})(NHR⁶)—, —(CR^{6a})(NHC(O)R⁶)—, —(CR^{6a})(NHSO₂R⁶)—, —(CR^{6a})(NHC(O)OR⁶)—, —(CR^{6a})(OC(O)R⁶)—, —(CR^{6a})(OC(O)NHR⁶)—, —(CR^{6a})═(CR^{6a})—, —C≡C—, —C(═NOR⁶)—, —C(O)—, —(CR^{6a})(OR⁶)—, —C(O)N(R⁶)—, —N(R⁶)C(O)—, —N(R⁶)S(O)—, —N(R⁶)S(O)₂— —OC(O)N(R⁶)—, N(R⁶)C(O)N(R^{6a})—, —NR⁶C(O)O—, —S(O)N(R⁶)—, —S(O)₂N(R⁶)—, —N(C(O)R⁶)S(O)—, —N(C(O)R⁶)S(O)₂—, —N(R⁶)S(O)N(R⁷)—, —N(R⁶)S(O)₂N(R⁷)—, —C(O)N(R⁶)C(O)—, —S(O)N(R⁷)C(O)—, —S(O)₂N(R⁶)C(O)—, —OS(O)N(R⁶)—, —OS(O)₂N(R⁶)—, —N(R⁶)S(O)O—, —N(R⁶)S(O)₂O—, —N(R⁶)S(O)C(O)—, —N(R⁶)S(O)₂C(O)—, —SON(C(O)R⁶)—, —SO₂N(C(O)R⁶)—, —N(R⁶)SON(R⁷)—, —N(R⁶)SO₂N(R⁷)—, —C(O)O—, —N(R⁶)P(OR⁷)O—, —N(R⁶)P(OR⁷)—, —N(R⁶)P(O)(OR⁷)O—, —N(R⁶)P(O)(OR⁷)—, —N(C(O)R⁶)P(OR⁷)O—, —N(C(O)R⁶)P(OR⁷)—, —N(C(O)R⁶)P(O)(OR⁷)O—, —N(C(O)R⁶)P(OR⁷)—, —(CR^{6a}R^{7a})S(O)—, —(CR^{6a}R^{7a})S(O)₂—, —(CR^{6a}R^{7a})N(C(O)OR⁷)—, —(CR^{6a}R^{7a})N(C(O)R⁷)—, —(CR^{6a}R^{7a})N(SO₂R⁷), —(CR^{6a}R^{7a})C(═NOR⁷)—, —(CR^{6a}R^{7a})C(O)—, —(CR^{6a}R^{7a})(CR⁶aa)(OR⁷)—, —(CR^{6a}R^{7a})C(O)N(R⁷)—, —(CR^{6a}R^{7a})N(R⁶)C(O)—, —(CR^{6a}R^{7a})N(R⁷)S(O)—, —(CR^{6a}R^{7a})N(R⁷)S(O)₂—, —(CR^{6a}R^{7a})OC(O)N(R⁷)—, —(CR^{6a}R^{7a})N(R⁷)C(O)N(R)—, —(CR^{6a}R^{7a})N(R⁷C(O)O—, —(CR^{6a}R^{7a})S(O)N(R⁷)—, —(CR^{6a}R^{7a})S(O)₂N(R⁷)—, —(CR^{6a}R^{7a})N(C(O)R⁷)S(O)—, —(CR^{6a}R^{7a})N(C(O)R⁷)S(O), —(CR^{6a}R^{7a})N(R⁷)S(O)N(R⁸)—, —(CR^{6a}R^{7a})N(R⁷)S(O)₂N(R)—, —(CR^{6a}R^{7a})C(O)N(R⁷)C(O)—, —(CR^{6a}R^{7a})S(O)N(R⁷)C(O), —(CR^{6a}R^{7a})S(O)₂N(R⁷)C(O)—, —(CR^{6a}R^{7a})OS(O)N(R⁷), —(CR^{6a}R^{7a})OS(O)₂N(R⁷)—, (CR^{6a}R^{7a})N(R⁷)S(O)O—, —(CR^{6a}R^{7a})N(R⁷)S(O)₂O, —(CR^{6a}R^{7a})N(R⁷)S(O)C(O)—, —(CR^{6a}R^{7a})N(R⁷)S(O)₂C(O)—, —(CR^{6a}R^{7a})SO₂N(C(O)R⁷), —(CR^{6a}R^{7a})SO₂N(C(O)R⁷)—, —(CR^{6a}R^{7a})N(R⁷)SON(R⁸)—, —(CR^{6a}R^{7a})N(R⁷)SO₂N(R⁸)—, —(CR^{6a}R^{7a})C(O)O—, —(CR^{6a}R^{7a})N(R⁷)P(OR⁸)O—, —(CR^{6a}R^{7a})N(R⁷)P(OR⁸)—, —(CR^{6a}R^{7a})N(R⁷)P(O)(OR)O—, —(CR^{6a}R^{7a})N(R⁷)P(O)(OR⁸)—, —(CR^{6a}R^{7a})N(C(O)R⁷)P(OR⁸)O—, —(CR^{6a}R^{7a})N(C(O)R⁷)P(OR⁸), —(CR^{6a}R^{7a})N(C(O)R⁷)P(O)(OR)O—, or —(CR^{6a}R^{7a})N(C(O)R⁷)P(OR⁵); R¹, R², R³, R⁴, R⁵, R⁶, R⁷, R⁸, R²², R^{22a}, R³³, and R^{33a} are, in each instance, each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹ substituents;
R^{6a}, R^{6aa}, and R^{7a} are, in each instance, each independently fluoro, trifluoromethyl, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G^{11a} substituents;
or in the case of —NR²R³(R⁴)_{j1b}, —NR³R⁴, —NR⁴R⁵, —NR^{2b}R^{3b}(R^{4b})_{j1b}, —NR^{3b}R^{4b}, —NR^{4b}R^{5b}, —NR⁹R¹⁰, —NR¹⁰R¹¹, —NR¹¹R¹², —NR²²R³³(R^{22a})ⱼ₁ₐ, —NR^{22a}R^{33a}, —NR³³R^{22a}, —NR⁶R¹, —NR⁷R¹, and —NR⁸R¹ then R² and R³, or R³ and R⁴, or R⁴ and R⁵, R^{2b} and R^{3b}, or R^{3b} and R^{4b}, or R^{4b} and R^{5b}, or R⁹ and R¹⁰, or R¹⁰ and R¹¹, or R¹¹ and R¹², or R²² and R³³, or R^{22a} and R^{33a}, or R³³ and R²², or R⁶ and R¹, or R⁷ and R¹, or R⁸ and R¹, respectively, are optionally taken together with the nitrogen atom to which they are attached to form a 3-10 membered saturated or unsaturated ring, wherein said ring is optionally substituted by one or more independent G¹¹¹ substituents and wherein said ring optionally includes one or more heteroatoms other than the nitrogen to which R² and R³, or R³ and R⁴, or R⁴ and R⁵, R^{2b} and R^{3b}, or R^{3b} and R^{4b}, or R^{4b} and R^{5b}, or R⁹ and R¹⁰, or R¹⁰ and R¹¹, or R¹¹ and R¹², or R²² and R³³, or R^{22a} and R^{33a}, or R³³ and R^{22a}, or R⁶ and R¹, or R⁷ and R¹, or R⁸ and R¹ are respectively attached;
or in the case of CR^{6a}R^{7a}, R^{6a} and R^{7a} can be taken together with the carbon to which they are attached to form a 3-10 membered saturated or unsaturated cycloalkyl or heterocycloalkyl ring, wherein said ring is optionally substituted by one or more independent G^{111a} substituents and wherein said ring optionally includes one or more heteroatoms;
G¹¹, G^{11a}, G¹¹¹, and G^{111a} are, in each instance, each independently halo, —CF₃, —OCF₃, —OR^{2b}, —NR^{2b}R^{3b}(R^{4b})_{j1b}, —C(═O)R^{2b}, —CO₂R^{2b}, CONR^{2b}R^{3b}, —NO₂, —CN, —S(O)_{j1b}R^{2b}, —SO₂NR^{2b}R^{3b}, —NR^{2b}C(═O)R^{3b}, —NR^{2b}C(═O)OR^{3b}, —NR^{2b}C(═O)NR^{3b}R^{4b}, —NR^{2b}S(O)_{j1b}R^{2b}, —C(═S)OR^{2b}, C(═O)SR^{2b}, —NR^{2b}C(═NR^{3b})N(R^{4b}R^{5b}, —NR^{2b}C(═NR^{3b})OR^{4b}, —NR^{2b}C(═NR^{3b})SR^{4b}, OC(═O)OR^{2b}, —C(═O)NR^{2b}R^{3b}, —OC(═O)SR^{2b}, SC(═O)OR^{2b}, —SC(═O)NR^{2b}R^{3b}, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR⁹, —NR⁹R¹⁰, —C(O)R⁹, —CO₂R⁹, —CONR⁹R¹⁰, —NO₂, —CN, —S(O)ⱼ₂ₐR⁹, —SO₂NR⁹R¹⁰, —NR⁹C(═O)R¹⁰, —NR⁹C(═O)OR¹⁰, —NR⁹C(═O)NR¹¹R¹⁰, —NR⁹S(O)ⱼ₂ₐR¹⁰, —C(═S)OR⁹, —C(═O)SR⁹, —NR⁹C(═NR¹⁰)NR¹¹R¹², —NR⁹C(═NR¹⁰)OR¹¹, —NR⁹C(═NR¹⁰)SR¹¹, —OC(═O)OR⁹, —OC(═O)NR⁹R¹⁰, —OC(═O)SR⁹, —SC(═O)OR⁹, —P(O)OR⁹OR¹⁰, or —SC(═O)NR⁹R¹⁰ substituents;
R^{2b}, R^{3b}, R^{4b}, R^{5b}, R⁹, R¹⁰, R¹¹ and R¹² are, in each instance, each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;
or R^{2b}, R^{3b}, R^{4b}, R^{5b}, R⁹, R¹⁰, R¹¹ and R¹² are, in each instance, each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents; and
j₁, j₁ₐ, j_{1b}, j₂, j₂ₐ, n, and m are, in each instance, each independently 0, 1, 2, or 3.

2. The compound according to claim 1, or a pharmaceutically acceptable salt thereof, wherein:
X¹ or X² are each —C(E¹)-;
X³ and X⁴ are combined to equal —C(E^{1a})=C(E¹)-;
X⁵ is NH; and
Q¹ is aryl-C₀₋₁₀alkyl optionally substituted by one or more independent G¹ substituents.

3. The compound according to claim 1, or a pharmaceutically acceptable salt thereof, wherein;
X¹ or X² are each —C(E¹)-;
X³ and X⁴ are combined to equal —C(E^{1a})=C(E¹)-;
X⁵ is NH; and
Q¹ is heterocyclyl-C₀₋₁₀alkyl optionally substituted by one or more independent G¹ substituents.

4. The compound according to claim 1, or a pharmaceutically acceptable salt thereof, wherein:
X¹ or X² are each —C(E¹)-;
X³ and X⁴ are combined to equal —C(E^{1a})=C(E¹)-;
X⁵ is NH; and
Q¹ is hetaryl-C₀₋₁₀alkyl optionally substituted by one or more independent G¹ substituents.

5. A composition comprising a compound according to claim 1, or a pharmaceutically acceptable salt thereof, and a pharmaceutically acceptable carrier.

6. A composition comprising a compound according to claim 1, or a pharmaceutically acceptable salt thereof; and an anti-neoplastic, anti-tumor, anti-angiogenic, or chemotherapeutic agent.

7. A composition comprising a compound according to claim 1, or a pharmaceutically acceptable salt thereof, and a cytotoxic or angiogenesis inhibiting cancer therapeutic agent.

8. A composition comprising a compound according to claim 1 selected from 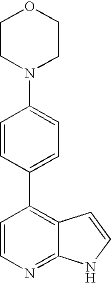 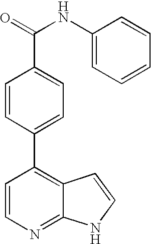 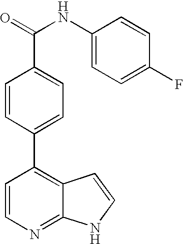 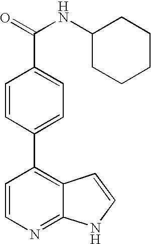 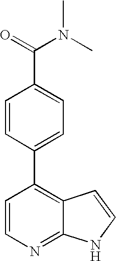 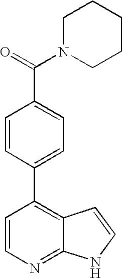 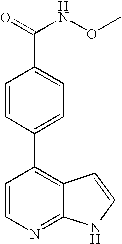 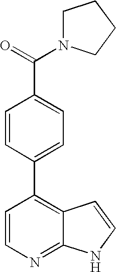 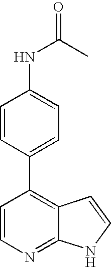 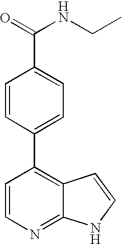 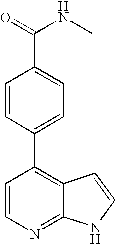 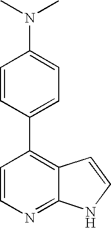 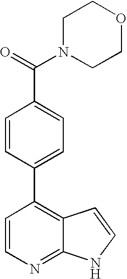 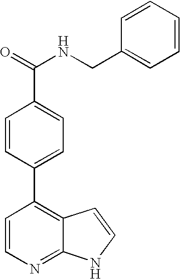 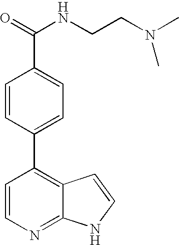 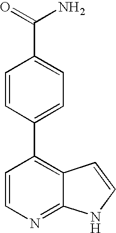 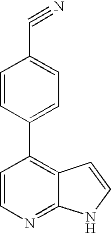 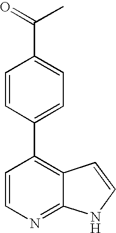 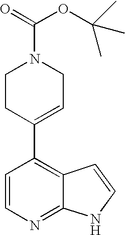 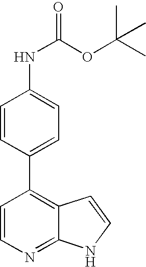 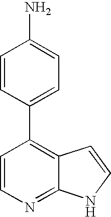 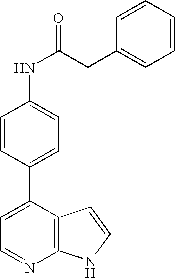 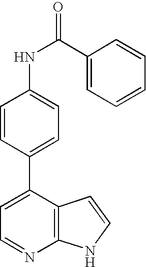 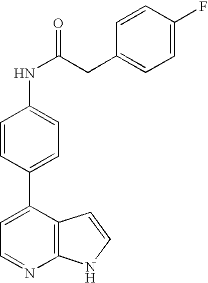 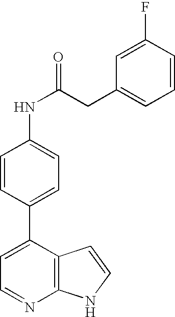 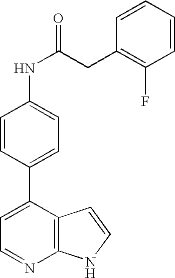 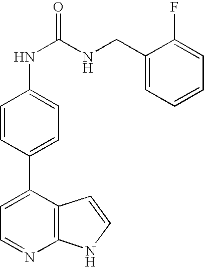 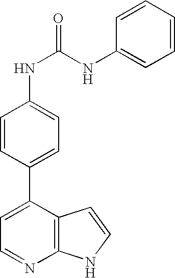 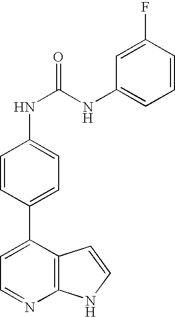 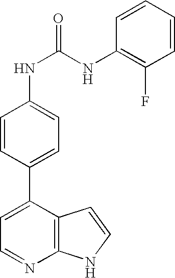 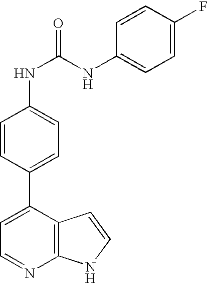 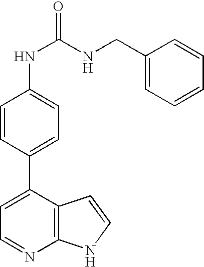 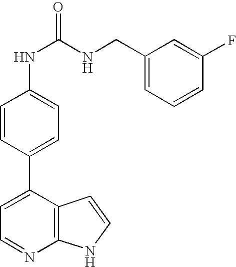 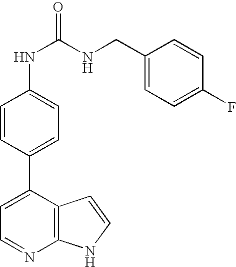 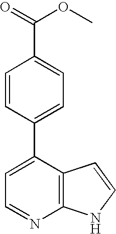 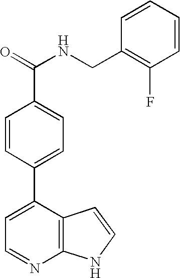 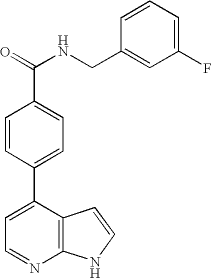 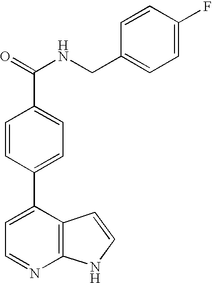 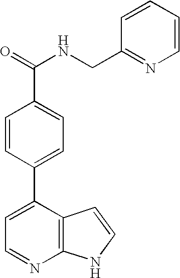 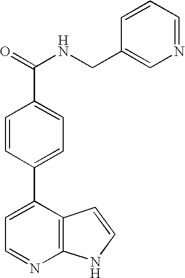 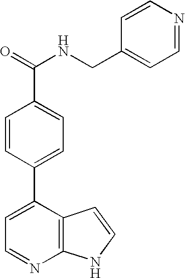 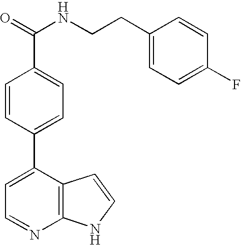 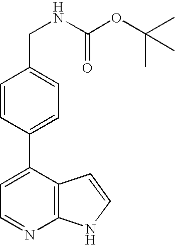 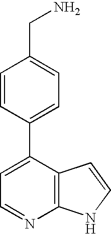 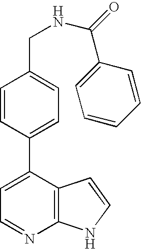 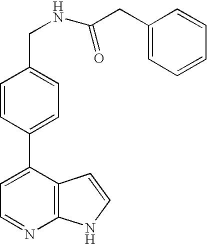 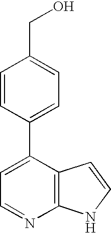 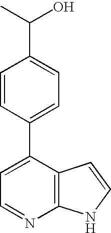 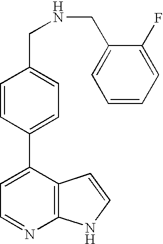 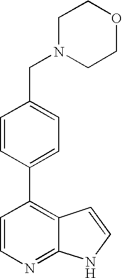 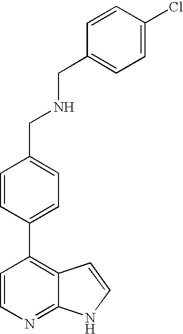 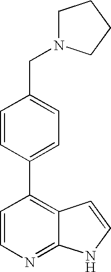 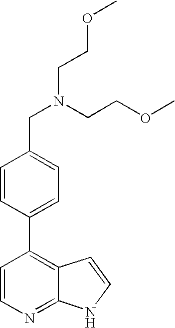 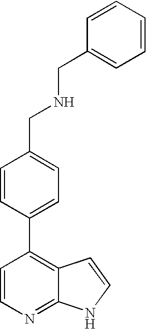 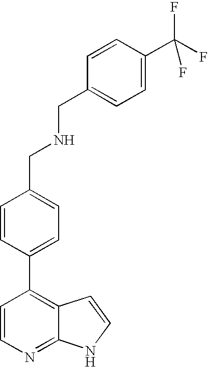 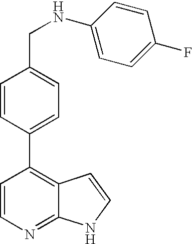 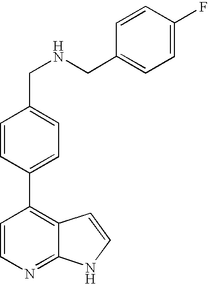 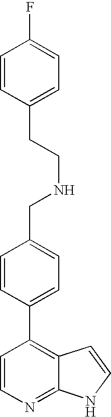 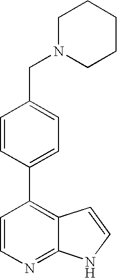 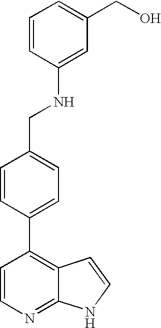 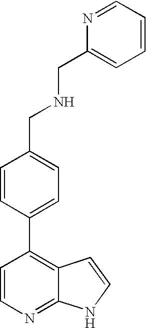 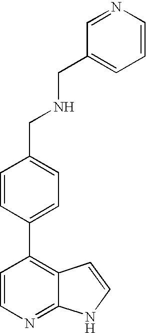 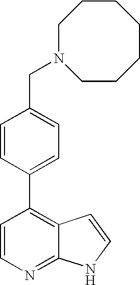 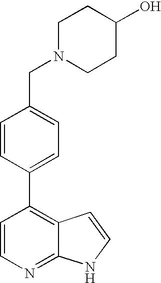 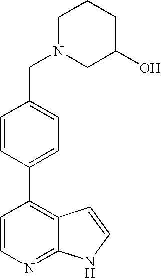 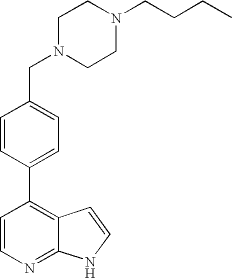 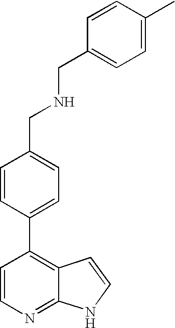 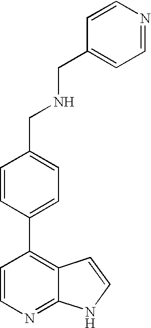 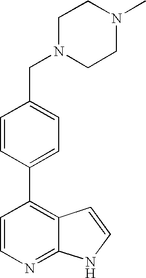 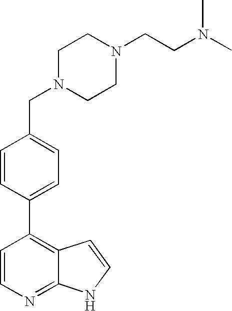 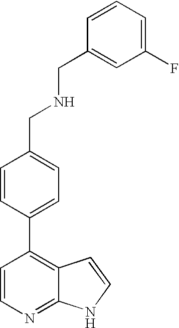 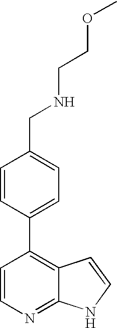 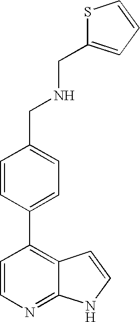 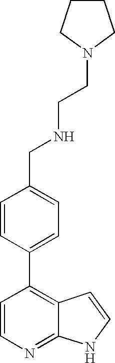 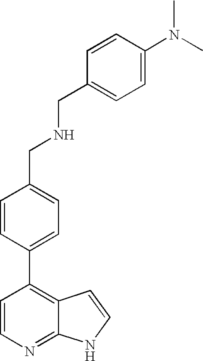 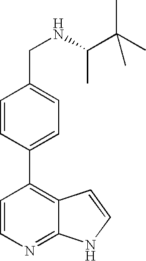 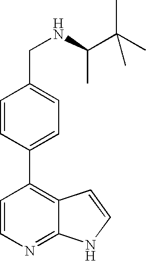 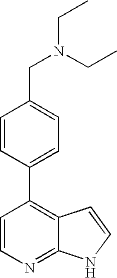 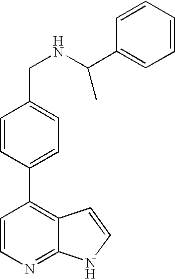 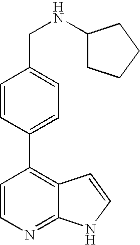 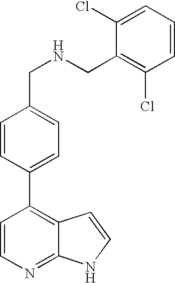 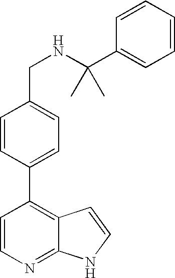 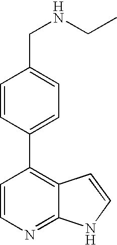 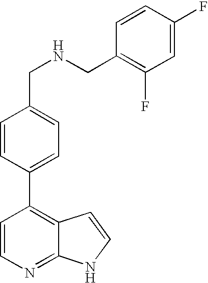 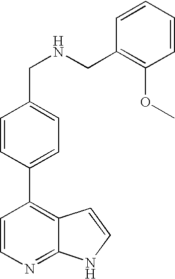 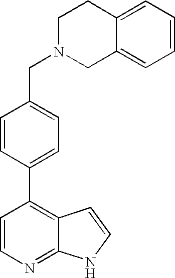 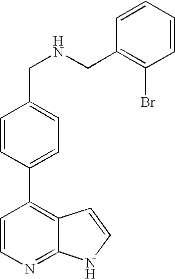 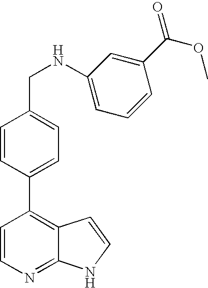 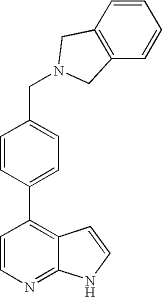 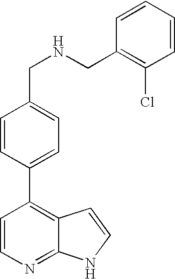 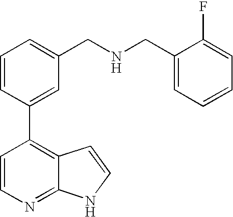 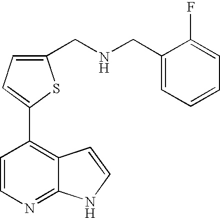 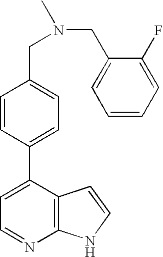 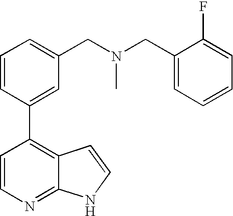
or a pharmaceutically acceptable salt thereof.

9. A composition comprising a compound of claim 1 selected from 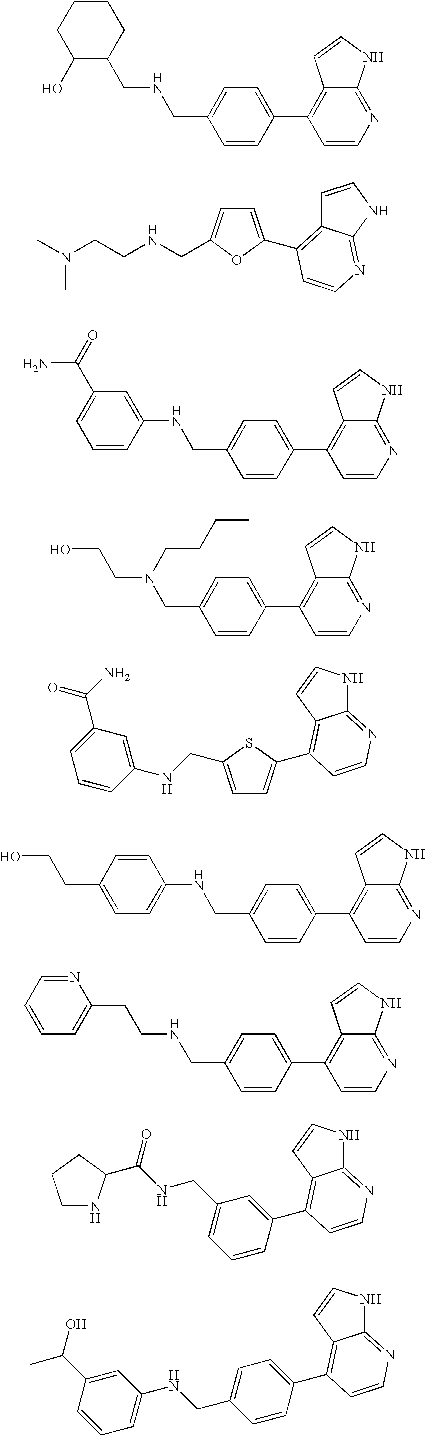 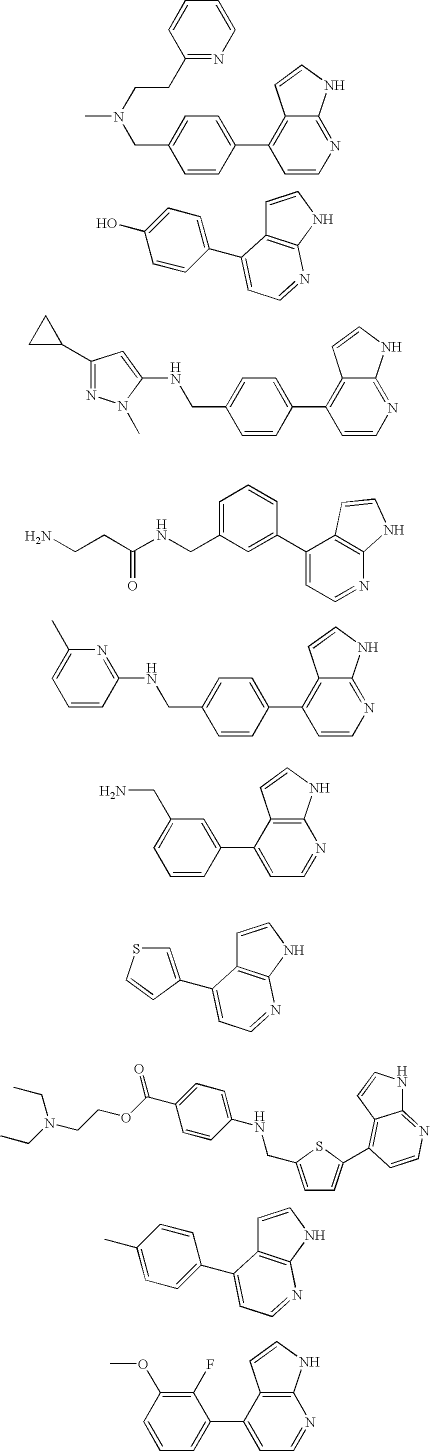 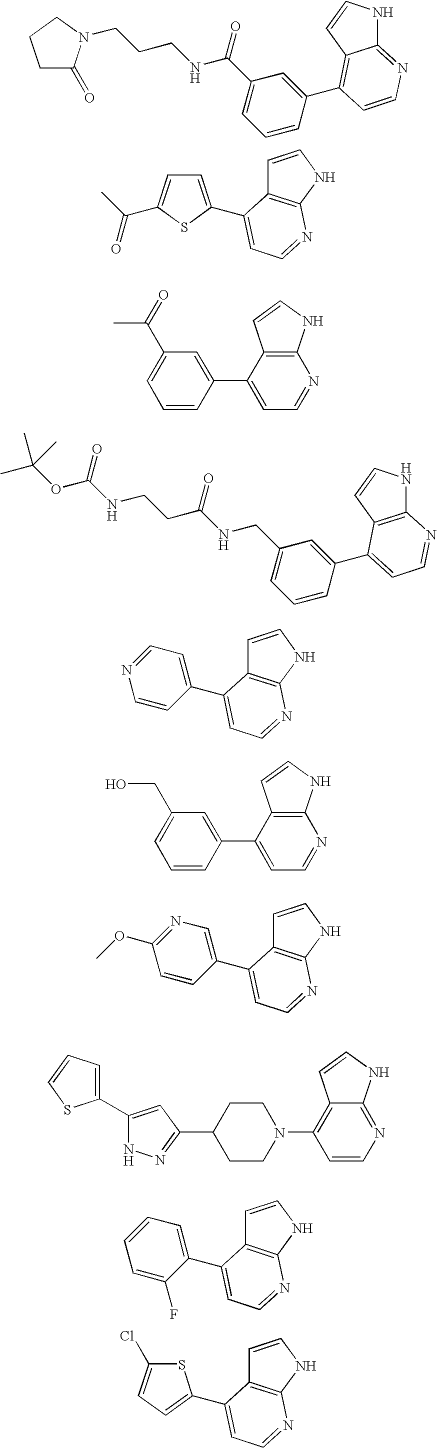 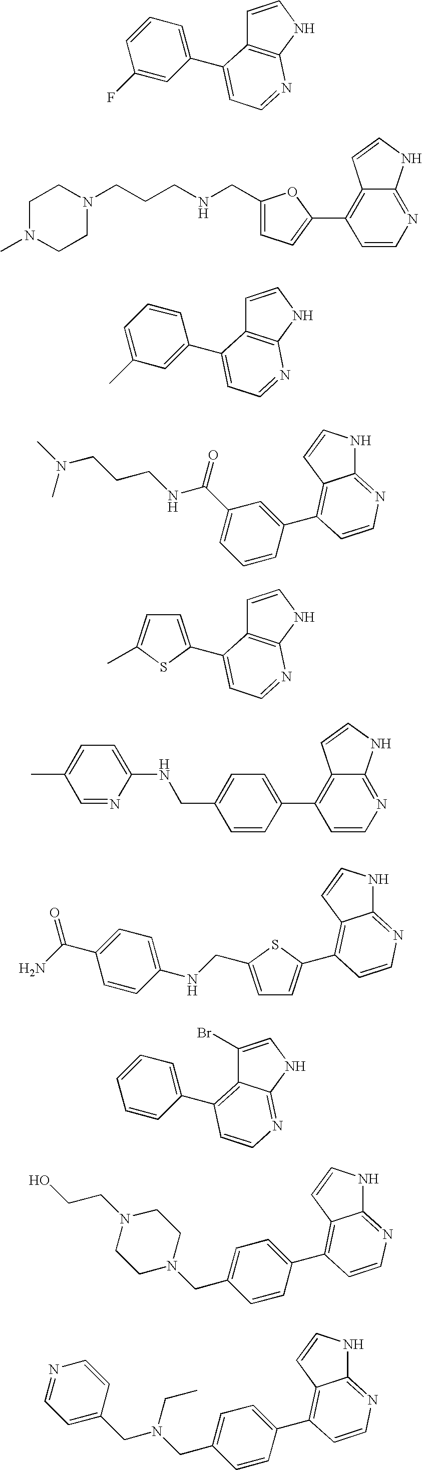 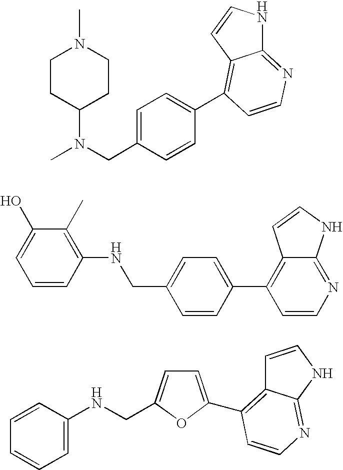
or a pharmaceutically acceptable salt thereof.

10. The compound according to claim 1 consisting of:
4-(4-morpholin-4-yl-phenyl)-1H-pyrrolo[2,3-b]pyridine;
N-phenyl-4-(1H-pyrrolo[2,3-b]pyridin-4-yl)-benzamide;
N-(4-fluoro-phenyl)-4-(1H-pyrrolo[2,3-b]pyridin-4-yl)-benzamide;
N-cyclohexyl-4-(1H-pyrrolo[2,3-b]pyridin-4-yl)-benzamide;
N,N-dimethyl-4-(1H-pyrrolo[2,3-b]pyridin-4-yl)-benzamide;
piperidin-1]-yl-[4-(1H-pyrrolo[2,3-b]pyridin-4-yl)-phenyl]-methanone;
N-methoxy-4-(1H-pyrrolo[2,3-b]pyridin-4-yl)-benzamide;
pyrrolidin-1-yl-[4-(1H-pyrrolo[2,3-b]pyridin-4-yl)-phenyl]-methanone;
N-[4-(1H-pyrrolo[2,3-b]pyridin-4-yl)-phenyl]-acetamide;
N-ethyl-4-(1H-pyrrolo[2,3-b]pyridin-4-yl)-benzamide;
N-methyl-4-(1H-pyrrolo[2,3-b]pyridin-4-yl)-benzamide;
dimethyl-[4-(1H-pyrrolo[2,3-b]pyridin-4-yl)-phenyl]-amine;
morpholin-4-yl-[4-(1H-pyrrolo[2,3-b]pyridin-4-yl)-phenyl]-methanone;
N-benzyl-4-(1H-pyrrolo[2,3-b]pyridin-4-yl)-benzamide;
N-(2-dimethylamino-ethyl)-4-(1H-pyrrolo[2,3-b]pyridin-4-yl)-benzamide;
4-(1H-pyrrolo[2,3-b]pyridin-4-yl)-benzamide;
4-(1H-pyrrolo[2,3-b]pyridin-4-yl)-benzonitrile;
1-[4-(1H-pyrrolo[2,3-b]pyridin-4-yl)-phenyl]-ethanone;
4-(1H-pyrrolo[2,3-b]pyridin-4-yl)-3,6-dihydro-2H-pyridine-1-carboxylic acid tert-butyl ester;
[4-(1H-pyrrolo[2,3-b]pyridin-4-yl)-phenyl]-carbamic acid tert-butyl ester;
4-(1H-pyrrolo[2,3-b]pyridin-4-yl)-phenylamine;
2-phenyl-N-[4-(1H-pyrrolo[2,3-b]pyridin-4-yl)-phenyl]-acetamide;
N-[4-(1H-pyrrolo[2,3-b]pyridin-4-yl)-phenyl]-benzamide;
2-(4-fluoro-phenyl)-N-[4-(1H-pyrrolo[2,3-b]pyridin-4-yl)-phenyl]-acetamide;
2-(3-fluoro-phenyl)-N-[4-(1H-pyrrolo[2,3-b]pyridin-4-yl)-phenyl]-acetamide;
2-(2-fluoro-phenyl)-N-[4-(1H-pyrrolo[2,3-b]pyridin-4-yl)-phenyl]-acetamide;
1-(2-fluoro-benzyl)-3-[4-(1H-pyrrolo[2,3-b]pyridin-4-yl)-phenyl]-urea;
1-phenyl-3-[4-(1H-pyrrolo[2,3-b]pyridin-4-yl)-phenyl]-urea;
1-(3-fluoro-phenyl)-3-[4-(1H-pyrrolo[2,3-b]pyridin-4-yl)-phenyl]-urea;
1-(2-fluoro-phenyl)-3-[4-(1H-pyrrolo[2,3-b]pyridin-4-yl)-phenyl]-urea;
or a pharmaceutically acceptable salt thereof.

11. The compound according to claim 1 consisting of:
1-(4-fluoro-phenyl)-3-[4-(1H-pyrrolo[2,3-b]pyridin-4-yl)-phenyl]-urea;
1-benzyl-3-[4-(1H-pyrrolo[2,3-b]pyridin-4-yl)-phenyl]-urea;
1-(3-fluoro-benzyl)-3-[4-(1H-pyrrolo[2,3-b]pyridin-4-yl)-phenyl]-urea;
1-(4-fluoro-benzyl)-3-[4-(1H-pyrrolo[2,3-b]pyridin-4-yl)-phenyl]-urea;
4-(1H-pyrrolo[2,3-b]pyridin-4-yl)-benzoic acid methyl ester;
N-(2-fluoro-benzyl)-4-(1H-pyrrolo[2,3-b]pyridin-4-yl)-benzamide;
N-(3-fluoro-benzyl)-4-(1H-pyrrolo[2,3-b]pyridin-4-yl)-benzamide;
N-(4-fluoro-benzyl)-4-(1H-pyrrolo[2,3-b]pyridin-4-yl)-benzamide;
N-pyridin-2-ylmethyl-4-(1H-pyrrolo[2,3-b]pyridin-4-yl)-benzamide;
N-Pyridin-3-ylmethyl-4-(1H-pyrrolo[2,3-b]pyridin-4-yl)-benzamide;
N-pyridin-4-ylmethyl-4-(1H-pyrrolo[2,3-b]pyridin-4-yl)-benzamide;
N-[2-(4-fluoro-phenyl)-ethyl]-4-(1H-pyrrolo[2,3-b]pyridin-4-yl)-benzamide;
[4-(1H-pyrrolo[2,3-b]pyridin-4-yl)-benzyl]-carbamic acid tert-butyl ester;
4-(1H-pyrrolo[2,3-b]pyridin-4-yl)-benzylamine;
N-[4-(1H-pyrrolo[2,3-b]pyridin-4-yl)-benzyl]-benzamide;
2-phenyl-N-[4-(1H-pyrrolo[2,3-b]pyridin-4-yl)-benzyl]-acetamide;
[4-(1H-pyrrolo[2,3-b]pyridin-4-yl)-phenyl]-methanol;
1-[4-(1H-pyrrolo[2,3-b]pyridin-4-yl)-phenyl]-ethanol;
(2-fluoro-benzyl)-[4-(1H-pyrrolo[2,3-b]pyridin-4-yl)-benzyl]-amine;
4-(4-morpholin-4-ylmethyl-phenyl)-1H-pyrrolo[2,3-b]pyridine;
(4-chloro-benzyl)-[4-(1H-pyrrolo[2,3-b]pyridin-4-yl)-benzyl]-amine;
4-(4-pyrrolidin-1-ylmethyl-phenyl)-1H-pyrrolo[2,3-b]pyridine;
bis-(2-methoxy-ethyl)-[4-(1H-pyrrolo[2,3-b]pyridin-4-yl)-benzyl]-amine;
benzyl-[4-(1H-pyrrolo[2,3-b]pyridin-4-yl)-benzyl]-amine;
[4-(1H-pyrrolo[2,3-b]pyridin-4-yl)-benzyl]-(4-trifluoromethyl-benzyl)-amine;
(4-fluoro-phenyl)-[4-(1H-pyrrolo[2,3-b]pyridin-4-yl)-benzyl]-amine;
(4-fluoro-benzyl)-[4-(1H-pyrrolo[2,3-b]pyridin-4-yl)-benzyl]-amine;
[2-(4-fluoro-phenyl)-ethyl]-[4-(1H-pyrrolo[2,3-b]pyridin-4-yl)-benzyl]-amine;
4-(4-piperidin-1-ylmethyl-phenyl)-1H-pyrrolo[2,3-b]pyridine;
{3-[4-(1H-pyrrolo[2,3-b]pyridin-4-yl)-benzylamino]-phenyl}-methanol;
pyridin-2-ylmethyl-[4-(1H-pyrrolo[2,3-b]pyridin-4-yl)-benzyl]-amine;
pyridin-3-ylmethyl-[4-(1H-pyrrolo[2,3-b]pyridin-4-yl)-benzyl]-amine;
4-(4-azocan-1-ylmethyl-phenyl)-1H-pyrrolo[2,3-b]pyridine;
1-[4-(1H-pyrrolo[2,3-b]pyridin-4-yl)-benzyl]-piperidin-4-ol;
1-[4-(1H-pyrrolo[2,3-b]pyridin-4-yl)-benzyl]-piperidin-3-ol;
4-[4-(4-butyl-piperazin-1-ylmethyl)-phenyl]-1H-pyrrolo[2,3-b]pyridine;
(4-methyl-benzyl)-[4-(1H-pyrrolo[2,3-b]pyridin-4-yl)-benzyl]-amine;
pyridin-4-ylmethyl-[4-(1H-pyrrolo[2,3-b]pyridin-4-yl)-benzyl]-amine;
4-[4-(4-methyl-piperazin-1-ylmethyl)-phenyl]-1H-pyrrolo[2,3-b]pyridine;
dimethyl-(2-{4-[4-(1H-pyrrolo[2,3-b]pyridin-4-yl)-benzyl]-piperazin-1-yl}-ethyl)-amine;
(3-fluoro-benzyl)-[4-(1H-pyrrolo[2,3-b]pyridin-4-yl)-benzyl]-amine;
(2-methoxy-ethyl)-[4-(1H-pyrrolo[2,3-b]pyridin-4-yl)-benzyl]-amine;
[4-(1H-pyrrolo[2,3-b]pyridin-4-yl)-benzyl]-thiophen-2-ylmethyl-amine;
(2-pyrrolidin-1-yl-ethyl)-[4-(1H-pyrrolo[2,3-b]pyridin-4-yl)-benzyl]-amine;
dimethyl-(4-{[4-(1H-pyrrolo[2,3-b]pyridin-4-yl)-benzylamino]-methyl}-phenyl)-amine;
(s)-[4-(1H-pyrrolo[2,3-b]pyridin-4-yl)-benzyl]-(1,2,2-trimethyl-propyl)-amine;
(R)-[4-(1H-pyrrolo[2,3-b]pyridin-4-yl)-benzyl]-(1,2,2-trimethyl-propyl)-amine;
diethyl-[4-(1H-pyrrolo[2,3-b]pyridin-4-yl)-benzyl]-amine;
(1-phenyl-ethyl)-[4-(1H-pyrrolo[2,3-b]pyridin-4-yl)-benzyl]-amine;
cyclopentyl-[4-(1H-pyrrolo[2,3-b]pyridin-4-yl)-benzyl]-amine;
(2,6-dichloro-benzyl)-[4-(1H-pyrrolo[2,3-b]pyridin-4-yl)-benzyl]-amine;
(1-methyl-1-phenyl-ethyl)-[4-(1H-pyrrolo[2,3-b]pyridin-4-yl)-benzyl]-amine;
ethyl-[4-(1H-pyrrolo[2,3-b]pyridin-4-yl)-benzyl]-amine;
(2,4-difluoro-benzyl)-[4-(1H-pyrrolo[2,3-b]pyridin-4-yl)-benzyl]-amine;
(2-methoxy-benzyl)-[4-(1H-pyrrolo[2,3-b]pyridin-4-yl)-benzyl]-amine;
2-[4-(1H-pyrrolo[2,3-b]pyridin-4-yl)-benzyl]-1,2,3,4-tetrahydro-isoquinoline;
(2-bromo-benzyl)-[4-(1H-pyrrolo[2,3-b]pyridin-4-yl)-benzyl]-amine;
3-[4-(1H-pyrrolo[2,3-b]pyridin-4-yl)-benzylamino]-benzoic acid methyl ester;
4-[4-(1,3-dihydro-isoindol-2-ylmethyl)-phenyl]-1H-pyrrolo[2,3-b]pyridine;
(2-chloro-benzyl)-[4-(1H-pyrrolo[2,3-b]pyridin-4-yl)-benzyl]-amine;
(2-fluoro-benzyl)-[3-(1H-pyrrolo[2,3-b]pyridin-4-yl)-benzyl]-amine;
(2-fluoro-benzyl)-[5-(1H-pyrrolo[2,3-b]pyridin-4-yl)-thiophen-2-ylmethyl]-amine;
(2-fluoro-benzyl)-methyl-[4-(1H-pyrrolo[2,3-b]pyridin-4-yl)-benzyl]-amine;
(2-fluoro-benzyl)-methyl-[3-(1H-pyrrolo[2,3-b]pyridin-4-yl)-benzyl]-amine;
2-{[4-(1H-pyrrolo[2,3-b]pyridin-4-yl)-benzylamino]-methyl}-cyclohexanol;
N,N-dimethyl-N′-[5-(1H-pyrrolo[2,3-b]pyridin-4-yl)-furan-2-ylmethyl]-ethane-1,2-diamine;
3-[4-(1H-pyrrolo[2,3-]pyridin-4-yl)-benzylamino]-benzamide;
2-{butyl-[4-(1H-pyrrolo[2,3-b]pyridin-4-yl)-benzyl]-amino}-ethanol;
3-{[5-(1H-pyrrolo[2,3-b]pyridin-4-yl)-thiophen-2-ylmethyl]-amino}-benzamide;
2-{4-[4-(1H-pyrrolo[2,3-b]pyridin-4-yl)-benzylamino]-phenyl-ethanol;
(2-pyridin-2-yl-ethyl)-[4-(1H-pyrrolo[2,3-b]pyridin-4-yl)-benzyl]-amine;
pyrrolidine-2-carboxylic acid 3-(1H-pyrrolo[2,3-b]pyridin-4-yl)-benzylamide;
1-{3-[4-(1H-pyrrolo[2,3-b]pyridin-4-yl)-benzylamino]-phenyl-ethanol;
4-(1H-pyrrolo[2,3-b]pyridin-4-yl)-phenol;
methyl-(2-pyridin-2-yl-ethyl)-[4-(1H-pyrrolo[2,3-b]pyridin-4-yl)-benzyl]-amine;
(5-cyclopropyl-2-methyl-2H-pyrazol-3-yl)-[4-(1H-pyrrolo[2,3-b]pyridin-4-yl)-benzyl]-amine;
(6-methyl-pyridin-2-yl)-[4-(1H-pyrrolo[2,3-b]pyridin-4-yl)-benzyl]-amine;
3-amino-N-[3-(1H-pyrrolo[2,3-b]pyridin-4-yl)-benzyl]-propionamide;
3-(1H-pyrrolo[2,3-b]pyridin-4-yl)-benzylamine;
4-thiophen-3-yl-1H-pyrrolo[2,3-b]pyridine;
4-{[5-(1H-pyrrolo[2,3-b]pyridin-4-yl)-thiophen-2-ylmethyl]-amino}-benzoic acid 2-diethylamino-ethyl ester;
4-p-tolyl-1H-pyrrolo[2,3-b]pyridine;
N-[3-(2-oxo-pyrrolidin-1-yl)-propyl]-3-(1H-pyrrolo[2,3-b]pyridin-4-yl)-benzamide;
4-(2-fluoro-3-methoxy-phenyl)-1H-pyrrolo[2,3-b]pyridine;
1-[5-(1H-pyrrolo[2,3-b]pyridin-4-yl)-thiophen-2-yl]-ethanone;
{2-[3-(1H-pyrrolo[2,3-b]pyridin-4-yl)-benzylcarbamoyl]-ethyl}-carbamic acid tert-butyl ester;
1-[3-(1H-pyrrolo[2,3-b]pyridin-4-yl)-phenyl]-ethanone;
4-pyridin-4-yl-1H-pyrrolo[2,3-b]pyridine;
[3-(1H-pyrrolo[2,3-b]pyridin-4-yl)-phenyl]-methanol;
4-(6-methoxy-pyridin-3-yl)-1H-pyrrolo[2,3-b]pyridine;
4-[4-(5-thiophen-2-yl-1H-pyrazol-3-yl)-piperidin-1-yl]-1H-pyrrolo[2,3-b]pyridine;
4-(2-fluoro-phenyl)-1H-pyrrolo[2,3-b]pyridine;
4-(5-chloro-thiophen-2-yl)-1H-pyrrolo[2,3-b]pyridine;
4-(3-fluoro-phenyl)-1H-pyrrolo[2,3-b]pyridine;
[3-(4-methyl-piperazin-1-yl)-propyl]-[5-(1H-pyrrolo[2,3 b]pyridin-4-yl)-furan-2-ylmethyl]-amine;
4-m-tolyl-1H-pyrrolo[2,3-b]pyridine;
N-(3-dimethylamino-propyl)-3-(1H-pyrrolo[2,3-b]pyridin-4-yl)-benzamide;
4-(5-methyl-thiophen-2-yl)-1H-pyrrolo[2,3-b]pyridine;
(5-methyl-pyridin-2-yl)-[4-(1H-pyrrolo[2,3-b]pyridin-4-yl)-benzyl]-amine;
4-{[5-(1H-pyrrolo[2,3-b]pyridin-4-yl)-thiophen-2-ylmethyl]-amino}-benzamide;
3-bromo-4-phenyl-1H-pyrrolo[2,3-b]pyridine;
2-{4-[4-(1H-pyrrolo[2,3-b]pyridin-4-yl)-benzyl]-piperazin-1-yl}-ethanol;
ethyl-pyridin-4-ylmethyl-[4-(1H-pyrrolo[2,3-b]pyridin-4-yl)-benzyl]-amine;
methyl-(1-methyl-piperidin-4-yl)-[4-(1H-pyrrolo[2,3-b]pyridin-4-yl)-benzyl]-amine;
2-methyl-3-[4-(1H-pyrrolo[2,3-b]pyridin-4-yl)-benzylamino]-phenol;
phenyl-[5-(1H-pyrrolo[2,3-b]pyridin-4-yl)-furan-2-ylmethyl]-amine;
1-[4-(3-bromo-1H-pyrrolo[2,3-b]pyridin-4-yl)-phenyl]-ethanone;
(5-ethyl-[1,3,4]thiadiazol-2-yl)-[3-(1H-pyrrolo[2,3-b]pyridin-4-yl)-benzyl]-amine;
1-(4-naphthalen-2-yl-1H-pyrrolo[2,3-b]pyridin-3-yl)-ethanone;
2-{4-[3-(1H-pyrrolo[2,3-b]pyridin-4-yl)-benzyl]-piperazin-1-yl}-ethanol;
2-{[3-(1H-pyrrolo[2,3-b]pyridin-4-yl)-benzylamino]-methyl}-cyclohexanol;
(1H-benzotriazol-5-yl)-[4-(1H-pyrrolo[2,3-b]pyridin-4-yl)-benzyl]-amine;
2-{4-[3-(1H-pyrrolo[2,3-b]pyridin-4-yl)-benzylamino]-phenyl}-ethanol;
4-[3-(1H-pyrrolo[2,3-b]pyridin-4-yl)-benzylamino]-benzamide;
(5-cyclopropyl-2-methyl-2H-pyrazol-3-yl)-[3-(1H-pyrrolo[2,3-b]pyridin-4-yl)-benzyl]-amine;
(6-methyl-pyridin-2-yl)-[3-(1H-pyrrolo[2,3-b]pyridin-4-yl)-benzyl]-amine;
1-[4-(3-Chloro-1H-pyrrolo[2,3-b]pyridin-4-yl)-phenyl]-ethanone;
4-benzo[1,3]dioxol-5-yl-3-bromo-1H-pyrrolo[2,3-b]pyridine;
N-(2,3-dihydroxy-propyl)-3-(1H-pyrrolo[2,3-b]pyridin-4-yl)-benzamide;
N-carbamoylmethyl-3-(1H-pyrrolo[2,3-b]pyridin-4-yl)-benzamide;
isoquinolin-5-yl-[5-(1H-pyrrolo[2,3-b]pyridin-4-yl)-thiophen-2-ylmethyl]-amine;
3-[3-(1H-pyrrolo[2,3-b]pyridin-4-yl)-benzylamino]-benzamide;
4-benzo[1,3]dioxol-5-yl-3-chloro-1H-pyrrolo[2,3-b]pyridine;
3-bromo-4-(4-vinyl-phenyl)-1H-pyrrolo[2,3-b]pyridine;
{3-[3-(1H-pyrrolo[2,3-b]pyridin-4-yl)-benzylamino]-phenyl}-methanol;
(E)-4-[4-(3-acetyl-1H-pyrrolo[2,3-b]pyridin-4-yl)-phenyl]-but-3-en-2-one;
3-[4-(1H-pyrrolo[2,3-b]pyridin-4-yl)-benzylamino]-benzoic acid;
3-chloro-4-phenyl-1H-pyrrolo[2,3-b]pyridine;
1-[4-(4-acetyl-phenyl)-1H-pyrrolo[2,3-b]pyridin-3-yl]-ethanone;
1-(4-phenyl-1H-pyrrolo[2,3-b]pyridin-3-yl)-ethanone;
1-[4-(3-fluoro-phenyl)-1H-pyrrolo[2,3-b]pyridin-3-yl]-ethanone;
4-biphenyl-4-yl-3-bromo-1H-pyrrolo[2,3-b]pyridine;
4-thiophen-2-yl-1H-pyrrolo[2,3-b]pyridine;
N-[2-(1H-imidazol-4-yl)-ethyl]-3-(1H-pyrrolo[2,3-b]pyridin-4-yl)-benzamide;
4-(4-methanesulfonyl-phenyl)-1H-pyrrolo[2,3-b]pyridine;
4-(3,5-difluoro-phenyl)-1H-pyrrolo[2,3-b]pyridine;
4-(6-methoxy-pyridin-2-yl)-1H-pyrrolo[2,3-b]pyridine;
4-(2-chloro-phenyl)-1H-pyrrolo[2,3-b]pyridine;
4-(3,4-dimethoxy-phenyl)-1H-pyrrolo[2,3-b]pyridine;
4-(2,3-difluoro-phenyl)-1H-pyrrolo[2,3-b]pyridine;
5-(1H-pyrrolo[2,3-b]pyridin-4-yl)-furan-2-carbaldehyde;
N,N-dimethyl-N′-[5-(1H-pyrrolo[2,3-b]pyridin-4-yl)-furan-2-ylmethyl]-benzene-1,4-diamine;
N-(2-dimethylamino-ethyl)-3-(1H-pyrrolo[2,3-b]pyridin-4-yl)-benzamide;
1-{3-[3-(1H-pyrrolo[2,3-b]pyridin-4-yl)-benzylamino]-phenyl}-ethanol;
(1-phenyl-ethyl)-[3-(1H-pyrrolo[2,3-b]pyridin-4-yl)-benzyl]-amine; and
1-[3-(1H-pyrrolo[2,3-b]pyridin-4-yl)-benzyl]-piperidine-3-carboxylic acid amide.
or a pharmaceutically acceptable salt thereof.

12. A method of treatment of hyperproliferative disorder comprising a step of administering an effective amount of the compound according to claim 1, or a pharmaceutically acceptable salt thereof.

13. The method of treatment according to claim 12, wherein the hyperproliferative disorder is breast cancer, lung cancer, non-small cell lung cancer, kidney cancer, renal cell carcinoma, prostate cancer, cancer of the blood, liver cancer, ovarian cancer, thyroid cancer, endometrial cancer, cancer of the GI tract, lymphoma, renal cell carcinoma, mantle cell lymphoma, or endometrial cancer.

14. A method of treatment of rheumatoid arthritis, hamartoma syndromes, transplant rejection, atherosclerosis, IBD, asthma, bacterial infection, viral infection, multiple sclerosis or immunosuppression diseases comprising a step of administering an effective amount of the compound according to claim 1, or a pharmaceutically acceptable salt thereof.
